SEQUENCE LISTING (38 sequences)

SEQ ID NO: 1 - protein, 16 aa, Homo sapiens
DFGLSRLMTGDTYTAH

SEQ ID NO: 2 - protein, 17 aa, Homo sapiens
  MISC_FEATURE (1)..(1): X is Myristolyl
XGQQPGKVLGDQRRPSL

SEQ ID NO: 3 - protein, 360 aa, Homo sapiens
MSQERPTFYRQELNKTIWEVPERYQNLSPVGSGAYGSVCAAFDTKTGLRVAVKKLSRPFQSIIHAKRTYRELRLLKHMKHENVIGLLDVFTPARSLEEFNDVYLVTHLMGADLNNIVKCQKLTDDHVQFLIYQILRGLKYIHSADIIHRDLKPSNLAVNEDCELKILDFGLARHTDDEMTGYVATRWYRAPEIMLNWMHYNQTVDIWSVGCIMAELLTGRTLFPGTDHIDQLKLILRLVGTPGAELLKKISSESARNYIQSLTQMPKMNFANVFIGANPLAVDLLEKMLVLDSDKRITAAQALAHAYFAQYHDPDDEPVADPYDQSFESRDLLIDEWKSLTYDEVISFVPPPLDQEEMES

SEQ ID NO: 4 - protein, 24 aa, Homo sapiens
DFGLARHTDDEMTGYVATRWYRTY

SEQ ID NO: 5 - protein, 21 aa, Homo sapiens
DFGSAKQLVKGEPNVSYICSK

SEQ ID NO: 6 - protein, 10 aa, Homo sapiens
GRPRTTSFAE

SEQ ID NO: 7 - protein, 21 aa, Homo sapiens
DFGMTRDIYETDYYRKGGKGL

SEQ ID NO: 8 - protein, 11 aa, Homo sapiens
PHFPQFSYSAS

SEQ ID NO: 9 - protein, 12 aa, Homo sapiens
TTSGSGSGLPLL

SEQ ID NO: 10 - protein, 1123 aa, Mus musculus
MLEICLKLVGCKSKKGLSSSSSCYLEEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTIYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVSAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKRGTRGGAGSMLQAPELPTKTRTCRRAAEQKDAPDTPELLHTKGLGESDALDSEPAVSPLLPRKERGPPDGSLNEDERLLPRDRKTNLFSALIKKKKKMAPTPPKRSSSFREMDGQPDRRGASEDDSRELCNGPPALTSDAAEPTKSPKASNGAGVPNGAFREPGNSGFRSPHMWKKSSTLTGSRLAAAEEESGMSSSKRFLRSCSASCMPHGARDTEWRSVTLPRDLPSAGKQFDSSTFGGHKSEKPALPRKRTSESRSEQVAKSTAMPLPGWLKKNEEAAEEGFKDTESSPGSSPPSLTPKLLRRQVTASPSSGLSHKEEATKGSASGMGTPATAEPAPPSNKVGLSKASSEEMRVRRHKHSSESPGRDKGRLAKLKPAPPPPPACTGKAGKPAQSPSQEAGEAGGPTKTKCTSLAMDAVNTDPTKAGPPGEGLRKPVPPSVPKPQSTAKPPGTPTSPVSTPSTAPAPSPLAGDQQPSSAAFIPLISTRVSLRKTRQPPERIASGTITKGVVLDSTEALCLAISRNSEQMASHSAVLEAGKNLYTFCVSYVDSIQQMRNKFAFREAINKLESNLRELQICPATASSGPAATQDFSKLLSSVKEISDIVRR

SEQ ID NO: 11 - protein, 1123 aa, Mus musculus; identical to SEQ ID NO: 10

SEQ ID NO: 12 - protein, 537 aa, Homo sapiens
  CHAIN (1)..(537): A chain for 1OPL
MGQQPGKVLGDQRRPSLPALHFIKGAGKRDSSRHGGPHCNVFVEHEALQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRNLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKENLYFQ

SEQ ID NO: 13 - protein, 537 aa, Homo sapiens; identical to SEQ ID NO: 12
  CHAIN (1)..(537): B Chain for 1OPL

SEQ ID NO: 14 - protein, 537 aa, Homo sapiens; identical to SEQ ID NO: 12

SEQ ID NO: 15 - protein, 420 aa, Homo sapiens
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKVFRPRTPPEAIALCSRLLEYTPTARLTPLEACAHSFFDELRDPNVKLPNGRDTPALFNFTTQELSSNPPLATILIPPHARIQAAASTPTNATAASDANTGDRGQTNNAASASASNST

SEQ ID NO: 16 - protein, 352 aa, Homo sapiens
  CHAIN (1)..(352): A Chain of 1H8F
SKVTTVVATPGQGPDRPQEVSYTDTKVIGNGSFGVVYQAKLCDSGELVAIKKVLQGKAFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFAFPQIKAHPWTKVFRPRTPPEAIALCSRLLEYTPTARLTPLEACAHSFFDELRDPNVKLPNGRDTPALFNFTTQELSSNPPLATILIPPHARIQA

SEQ ID NO: 17 - protein, 352 aa, Homo sapiens; identical to SEQ ID NO: 16
  CHAIN (1)..(352): B Chain of 1H8F

SEQ ID NO: 18 - protein, 420 aa, Homo sapiens; identical to SEQ ID NO: 15

SEQ ID NO: 19 - protein, 306 aa, Homo sapiens
  CHAIN (1)..(306): A Chain from 1GAG
VFPSSVFVPDEWEVSREKITLLRELGQGSFGMVYEGNARDIIKGEAETRVAVKTVNESASLRERIEFLNEASVMKGFTCHHVVRLLGVVSKGQPTLVVMELMAHGDLKSYLRSLRPEAENNPGRPPPTLQEMIQMAAEIADGMAYLNAKKFVHRDLAARNCMVAHDFTVKIGDFGMTRDIXETDXXRKGGKGLLPVRWMAPESLKDGVFTTSSDMWSFGVVLWEITSLAEQPYQGLSNEQVLKFVMDGGYLDQPDNCPERVTDLMRMCWQFNPKMRPTFLEIVNLLKDDLHPSFPEVSFFHSEENK

SEQ ID NO: 20 - protein, 13 aa, Homo sapiens
  CHAIN (1)..(13): B Chain of 1IRK
PATGDFMNMSPVG

SEQ ID NO: 21 - protein, 307 aa, Homo sapiens
IVFPSSVFVPDEWEVSREKITLLRELGQGSFGMVYEGNARDIIKGEAETRVAVKTVNESASLRERIEFLNEASVMKGFTCHHVVRLLGVVSKGQPTLVVMELMAHGDLKSYLRSLRPEAENNPGRPPPTLQEMIQMAAEIADGMAYLNAKKFVHRDLAARNCMVAHDFTVKIGDFGMTRDIYETDYYRKGGKGLLPVRWMAPESLKDGVFTTSSDMWSFGVVLWEITSLAEQPYQGLSNEQVLKFVMDGGYLDQPDNCPERVTDLMRMCWQFNPKMRPTFLEIVNLLKDDLHPSFPEVSFFHSEENK

SEQ ID NO: 22 - protein, 315 aa, Homo sapiens
  CHAIN (1)..(315): A Chain of 1GZK
KVTMNDFDYLKLLGKGTFGKVILVREKATGRYYAMKILRKEVIIAKDEVAHTVTESRVLQNTRHPFLTALKYAFQTHDRLCFVMEYANGGELFFHLSRERVFTEERARFYGAEIVSALEYLHSRDVVYRDIKLENLMLDKDGHIKITDFGLCKEGISDGATMKTFCGTPEYLAPEVLEDNDYGRAVDWWGLGVVMYEMMCGRLPFYNQDHERLFELILMEEIRFPRTLSPEAKSLLAGLLKKDPKQRLGGGPSDAKEVMEHRFFLSINWQDVVQKKLLPPFKPQVTSEVDTRYFDDEFTAQSITITPPDRYDSLG

SEQ ID NO: 23 - protein, 315 aa, Homo sapiens; identical to SEQ ID NO: 22
  CHAIN (1)..(315): A Chain of 1GZO

SEQ ID NO: 24 - protein, 335 aa, Homo sapiens
  CHAIN (1)..(335): A Chain of 1GZN
KVTMNDFDYLKLLGKGTFGKVILVREKATGRYYAMKILRKEVIIAKDEVAHTVTESRVLQNTRHPFLTALKYAFQTHDRLCFVMEYANGGELFFHLSRERVFTEERARFYGAEIVSALEYLHSRDVVYRDIKLENLMLDKDGHIKITDFGLCKEGISDGATMKTFCGTPEYLAPEVLEDNDYGRAVDWWGLGVVMYEMMCGRLPFYNQDHERLFELILMEEIRFPRTLSPEAKSLLAGLLKKDPKQRLGGGPSDAKEVMEHRFFLSINWQDVVQKKLLPPFKPQVTSEVDTRYFDDEFTAQSITITPPDRYDSLGLLELDQRTHFPQFSYSASIR

SEQ ID NO: 25 - protein, 503 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 26 - protein, 342 aa, Homo sapiens
EDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 27 - protein, 350 aa, Homo sapiens
  CHAIN (1)..(350): A Chain of 1O9U
SKVTTVVATPGQGPDRPQEVSYTDTKVIGNGSFGVVYQAKLCDSGELVAIKKVLQGKAFKNRELQIMRKLDHCNIVRLRYFFYSSGEKKDEVYLNLVLDYVPATVYRVARHYSRAKQTLPVIYVKLYMYQLFRSLAYIHSFGICHRDIKPQNLLLDPDTAVLKLCDFGSAKQLVRGEPNVSXICSRYYRAPELIFGATDYTSSIDVWSAGCVLAELLLGQPIFPGDSGVDQLVEIIKVLGTPTREQIREMNPNYTEFAFPQIKAHPWTKVFRPRTPPEAIALCSRLLEYTPTARLTPLEACAHSFFDELRDPNVKLPNGRDTPALFNFTTQELSSNPPLATILIPPHARI

SEQ ID NO: 28 - protein, 18 aa, Homo sapiens
  CHAIN (1)..(18): B Chain of 1O9U
VEPQKFAEELIHRLEAVQ

SEQ ID NO: 29 - protein, 292 aa, Homo sapiens
GAMDPSSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKRG

SEQ ID NO: 30 - protein, 360 aa, Homo sapiens; identical to SEQ ID NO: 3

SEQ ID NO: 31 - protein, 414 aa, Homo sapiens
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKVFRPRTPPEAIALCSRLLEYTPTARLTPLEACAHSFFDELRDPNVKLPNGRDTPALFNFTTQELSSNPPLATILIPPHARIQAAASTPTNATAASDANTGDRGQTNNAASA

SEQ ID NO: 32 - protein, 367 aa, Homo sapiens
KVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKVFRPRTPPEAIALCSRLLEYTPTARLTPLEACAHSFFDELRDPNVKLPNGRDTPALFNFTTQELSSNPPLATILIPPHARIQAAASTPTN

SEQ ID NO: 33 - protein, 2029 aa, Homo sapiens
MVDPVGFAEAWKAQFPDSEPPRMELRSVGDIEQELERCKASIRRLEQEVNQERFRMIYLQTLLAKEKKSYDRQRWGFRRAAQAPDGASEPRASASRPQPAPADGADPPPAEEPEARPDGEGSPGKARPGTARRPGAAASGERDDRGPPASVAALRSNFERIRKGHGQPGADAEKPFYVNVEFHHERGLVKVNDKEVSDRISSLGSQAMQMERKKSQHGAGSSVGDASRPPYRGRSSESSCGVDGDYEDAELNPRFLKDNLIDANGGSRPPWPPLEYQPYQSIYVGGMMEGEGKGPLLRSQSTSEQEKRLTWPRRSYSPRSFEDCGGGYTPDCSSNENLTSSEEDFSSGQSSRVSPSPTTYRMFRDKSRSPSQNSQQSFDSSSPPTPQCHKRHRHCPVVVSEATIVGVRKTGQIWPNDGEGAFHGDADGSFGTPPGYGCAADRAEEQRRHQDGLPYIDDSPSSSPHLSSKGRGSRDALVSGALESTKASELDLEKGLEMRKWVLSGILASEETYLSHLEALLLPMKPLKAAATTSQPVLTSQQIETIFFKVPELYEIHKEFYDGLFPRVQQWSHQQRVGDLFQKLASQLGVYRAFVDNYGVAMEMAEKCCQANAQFAEISENLRARSNKDAKDPTTKNSLETLLYKPVDRVTRSTLVLHDLLKHTPASHPDHPLLQDALRISQNFLSSINEEITPRRQSMTVKKGEHRQLLKDSFMVELVEGARKLRHVFLFTELLLCTKLKKQSGGKTQQYDCKWYIPLTDLSFQMVDELEAVPNIPLVPDEELDALKIKISQIKSDIQREKRANKGSKATERLKKKLSEQESLLLLMSPSMAFRVHSRNGKSYTFLISSDYERAEWRENIREQQKKCFRSFSLTSVELQMLTNSCVKLQTVHSIPLTINKEDDESPGLYGFLNVIVHSATGFKQSSLQRPVASDFEPQGLSEAARWNSKENLLAGPSENDPNLFVALYDFVASGDNTLSITKGEKLRVLGYNHNGEWCEAQTKNGQGWVPSNYITPVNSLEKHSWYHGPVSRNAAEYLLSSGINGSFLVRESESSPGQRSISLRYEGRVYHYRINTASDGKLYVSSESRFNTLAELVHHHSTVADGLITTLHYPAPKRNKPTVYGVSPNYDKWEMERTDITMKHKLGGGQYGEVYEGVWKKYSLTVAVKTLKEDTMEVEEFLKEAAVMKEIKHPNLVQLLGVCTREPPFYIITEFMTYGNLLDYLRECNRQEVNAVVLLYMATQISSAMEYLEKKNFIHRDLAARNCLVGENHLVKVADFGLSRLMTGDTYTAHAGAKFPIKWTAPESLAYNKFSIKSDVWAFGVLLWEIATYGMSPYPGIDLSQVYELLEKDYRMERPEGCPEKVYELMRACWQWNPSDRPSFAEIHQAFETMFQESSISDEVEKELGKQGVRGAVSTLLQAPELPTKTRTSRRAAEHRDTTDVPEMPHSKGQGESDPLDHEPAVSPLLPRKERGPPEGGLNEDERLLPKDKKTNLFSALIKKKKKTAPTPPKRSSSFREMDGQPERRGAGEEEGRDISNGALAFTPLDTADPAKSPKPSNGAGVPNGALRESGGSGFRSPHLWKKSSTLTSSRLATGEEEGGGSSSKRFLRSCSASCVPHGAKDTEWRSVTLPRDLQSTGRQFDSSTFGGHKSEKPALPRKRAGENRSDQVTRGTVTPPPRLVKKNEEAADEVFKDIMESSPGSSPPNLTPKPLRRQVTVAPASGLPHKEEAGKGSALGTPAAAEPVTPTSKAGSGAPGGTSKGPAEESRVRRHKHSSESPGRDKGKLSRLKPAPPPPPAASAGKAGGKPSQSPSQEAAGEAVLGAKTKATSLVDAVNSDAAKPSQPGEGLKKPVLPATPKPQSAKPSGTPISPAPVPSTLPSASSALAGDQPSSTAFIPLISTRVSLRKTRQPPERIASGAITKGVVLDSTEALCLAISRNSEQMASHSAVLEAGKNLYTFCVSYVDSIQQMRNKFAFREAINKLENNLRELQICPATAGSGPAATQDFSKLLSSVKEISDIVQR

SEQ ID NO: 34 - protein, 1382 aa, Homo sapiens
MGTGGRRGAAAAPLLVAVAALLLGAAGHLYPGEVCPGMDIRNNLTRLHELENCSVIEGHLQILLMFKTRPEDFRDLSFPKLIMITDYLLLFRVYGLESLKDLFPNLTVIRGSRLFFNYALVIFEMVHLKELGLYNLMNITRGSVRIEKNNELCYLATIDWSRILDSVEDNHIVLNKDDNEECGDICPGTAKGKTNCPATVINGQFVERCWTHSHCQKVCPTICKSHGCTAEGLCCHSECLGNCSQPDDPTKCVACRNFYLDGRCVETCPPPYYHFQDWRCVNFSFCQDLHHKCKNSRRQGCHQYVIHNNKCIPECPSGYTMNSSNLLCTPCLGPCPKVCHLLEGEKTIDSVTSAQELRGCTVINGSLIINIRGGNNLAAELEANLGLIEEISGYLKIRRSYALVSLSFFRKLRLIRGETLEIGNYSFYALDNQNLRQLWDWSKHNLTTTQGKLFFHYNPKLCLSEIHKMEEVSGTKGRQERNDIALKTNGDKASCENELLKFSYIRTSFDKILLRWEPYWPPDFRDLLGFMLFYKEAPYQNVTEFDGQDACGSNSWTVVDIDPPLRSNDPKSQNHPGWLMRGLKPWTQYAIFVKTLVTFSDERRTYGAKSDIIYVQTDATNPSVPLDPISVSNSSSQIILKWKPPSDPNGNITHYLVFWERQAEDSELFELDYCLKGLKLPSRTWSPPFESEDSQKHNQSEYEDSAGECCSCPKTDSQILKELEESSFRKTFEDYLHNVVFVPRKTSSGTGAEDPRPSRKRRSLGDVGNVTVAVPTVAAFPNTSSTSVPTSPEEHRPFEKVVNKESLVISGLRHFTGYRIELQACNQDTPEERCSVAAYVSARTMPEAKADDIVGPVTHEIFENNVVHLMWQEPKEPNGLIVLYEVSYRRYGDEELHLCVSRKHFALERGCRLRGLSPGNYSVRIRATSLAGNGSWTEPTYFYVTDYLDVPSNIAKIIIGPLIFVFLFSVVIGSIYLFLRKRQPDGPLGPLYASSNPEYLSASDVFPCSVYVPDEWEVSREKITLLRELGQGSFGMVYEGNARDIIKGEAETRVAVKTVNESASLRERIEFLNEASVMKGFTCHHVVRLLGVVSKGQPTLVVMELMAHGDLKSYLRSLRPEAENNPGRPPPTLQEMIQMAAEIADGMAYLNAKKFVHRDLAARNCMVAHDFTVKIGDFGMTRDIYETDYYRKGGKGLLPVRWMAPESLKDGVFTTSSDMWSFGVVLWEITSLAEQPYQGLSNEQVLKFVMDGGYLDQPDNCPERVTDLMRMCWQFNPKMRPTFLEIVNLLKDDLHPSFPEVSFFHSEENKAPESEELEMEFEDMENVPLDRSSHCQREEAGGRDGGSSLGFKRSYEEHIPYTHMNGGKKNGRILTLPRSNPS

SEQ ID NO: 35 - protein, 290 aa, Homo sapiens
EKITLLRELGQGSFGMVYEGNARDIIKGEAETRVAVKTVNESASLRERIEFLNEASVMKGFTCHHVVRLLGVVSKGQPTLVVMELMAHGDLKSYLRSLRPEAENNPGRPPPTLQEMIQMAAEIADGMAYLNAKKFVHRDLAARNCMVAHDFTVKIGDFGMTRDIYETDYYRKGGKGLLPVRWMAPESLKDGVFTTSSDMWSFGVVLWEITSLAEQPYQGLSNEQVLKFVMDGGYLDQPDNCPERVTDLMRMCWQFNPKMRPTFLEIVNLLKDDLHPSFPEVSFFHSEENK

SEQ ID NO: 36 - protein, 480 aa, Homo sapiens
MSDVAIVKEGWLHKRGEYIKTWRPRYFLLKNDGTFIGYKERPQDVDQREAPLNNFSVAQCQLMKTERPRPNTFIIRCLQWTTVIERTFHVETPEEREEWTTAIQTVADGLKKQEEEEMDFRSGSPSDNSGAEEMEVSLAKPKHRVTMNEFEYLKLLGKGTFGKVILVKEKATGRYYAMKILKKEVIVAKDEVAHTLTENRVLQNSRHPFLTALKYSFQTHDRLCFVMEYANGGELFFHLSRERVFSEDRARFYGAEIVSALDYLHSEKNVVYRDLKLENLMLDKDGHIKITDFGLCKEGIKDGATMKTFCGTPEYLAPEVLEDNDYGRAVDWWGLGVVMYEMMCGRLPFYNQDHEKLFELILMEEIRFPRTLGPEAKSLLSGLLKKDPKQRLGGGSEDAKEIMQHRFFAGIVWQHVYEKKLSPPFKPQVTSETDTRYFDEEFTAQMITITPPDQDDSMECVDSERRPHFPQFSYSASSTA

SEQ ID NO: 37 - protein, 335 aa, Homo sapiens; identical to SEQ ID NO: 24

SEQ ID NO: 38 - protein, 390 aa, Homo sapiens
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

(19) United States
(12) Patent Application Publication
(10) Pub. No.: US 2004/0171075 A1
(43) Pub. Date: Sep. 2, 2004

(54) Modulation of protein functionalities

(76) Inventors: Daniel L. Flynn, Lawrence, KS (US); Peter A. Petillo, Arlington, MA (US)

Correspondence Address: HOVEY WILLIAMS LLP, 2405 GRAND BLVD., SUITE 400, KANSAS CITY, MO, 64108, US

(21) Appl. No.: 10/746,545

(22) Filed: Dec. 24, 2003

Related U.S. Application Data
(60) Provisional application No. 60/437,487, filed on Dec. 31, 2002.
(60) Provisional application No. 60/437,403, filed on Dec. 31, 2002.
(60) Provisional application No. 60/437,415, filed on Dec. 31, 2002.
(60) Provisional application No. 60/437,304, filed on Dec. 31, 2002.
(60) Provisional application No. 60/463,804, filed on Apr. 18, 2003.

(51) Int. Cl. G01N 33/53; G06F 19/00; G01N 33/48; G01N 33/50

(52) U.S. Cl. 435/7.1; 702/19

(57) ABSTRACT

New methods for the rational identification of molecules capable of interacting with specific naturally occurring proteins are provided, in order to yield new pharmacologically important compounds and treatment modalities. Broadly, the method comprises the steps of identifying a switch control ligand forming a part of a particular protein of interest, and also identifying a complemental switch control pocket forming a part of the protein and which interacts with said switch control ligand. The ligand interacts in vivo with the pocket to regulate the conformation and biological activity of the protein such that the protein assumes a first conformation and a first biological activity upon the ligand-pocket interaction, and assumes a second, different conformation and biological activity in the absence of the ligand-pocket interaction. Next, respective samples of said protein in the first and second conformations are provided, and these are screened against one or more candidate molecules by contacting the molecules and the samples. Thereupon, small molecules which bind with the protein at the region of the pocket may be identified. Novel protein-modulator adducts and methods of altering protein activity are also provided.

### CROSS-REFERENCE TO RELATED APPLICATIONS

This application claims the benefit of provisional applications entitled Process For Modulating Protein. Function, S/N 60/437,487 filed Dec. 31, 2002, Anti-Cancer Medicaments, S/N 60/437,403 filed Dec. 31, 2002, Anti-Inflammatory Medicaments, S/N 60/437,415 filed Dec. 31, 2002, Anti-Inflammatory Medicaments, S/N 60/437,304 filed Dec. 31, 2002, and Medicaments For the Treatment of Neurodegenerative Disorders or Diabetes, S/N 60/463,804 filed Apr. 18, 2003. Each of these applications is incorporated by reference herein.

### BACKGROUND OF THE INVENTION

1. Field of the Invention

The present invention is broadly concerned with new, rationalized methods of identifying molecules which serve as protein activity modulators, as well as new protein-modulator adducts. More particularly, the invention is concerned with such methods and adducts which, in preferred forms, make use of a mechanism of protein conformation change involving interaction between switch control ligands and complemental switch control pockets.

2. Description of the Prior Art

Basic research has recently provided the life sciences community with an unprecedented volume of information of the human genetic code, and the proteins that are produced by it. In 2001, the complete sequence of the human genome was reported (Lander, E. S. et al., Initial Sequencing and Analysis of the Human Genome; Nature (2001) 409:860; Venter, J. C. et al., The Sequence of the Human Genome, Science (2001) 291:1304). The global research community is now classifying the 50,000+ proteins that are encoded by this genetic sequence, and more importantly, it is attempting to identify those proteins that are causative of major, under-treated human diseases. Despite the wealth of information that the human genome and its proteins are providing, particularly in the area of conformational control of protein function, the methodology and strategy by which the pharmaceutical industry sets about to develop small molecule therapeutics has not significantly advanced beyond using native protein binding sites for binding to small molecule therapeutic agents. These native sites are normally used by proteins to perform essential cellular functions by binding to and processing natural substrates or transducing signals from natural ligands. Because these native sites are used broadly by many other proteins within protein families, drugs which interact with them are often plagued by lack of selectivity and, as a consequence, insufficient therapeutic windows to achieve maximum efficacy. Side effects and toxicities are revealed in such small molecules, either during preclinical discovery, clinical trials, or later in the marketplace. Side effects and toxicities continue to be a major reason for the high attrition rate seen within the drug development process. For the kinase protein family of proteins, interactions at these native sites have been recently reviewed: see J. Dumas, Emerging Pharmacophores: 1997-2000, Expert Opinion on Therapeutic Patents (2001) 11: 405-429; J. Dumas, Editor, Current Topics in Medicinal Chemistry (2002) 2: issue 9.

It is known that proteins are flexible, and this flexibility has been reported and utilized with the discovery of the small molecules which bind to alternative, flexible active sites with proteins. For review of this topic, see Teague, Nature Reviews/Drug Discovery, Vol. 2, pp. 527-541 (2003). See also, Wu et al., Structure, Vol. 11, pp. 399-410 (2003). However these reports focus on small molecules which bind only to proteins at the protein natural active sites. Peng et al., Bio. Organic and Medicinal Chemistry Ltrs., Vol. 13, pp. 3693-3699 (2003), and Schindler, et al., Science, Vol. 289, p. 1938 (2000) describe inhibitors of abl kinase. These inhibitors are identified in WO Publication No. 2002/034727. This class of inhibitors binds to the ATP active site while also binding in a mode that induces movement of the kinase catalytic loop. Pargellis et al., Nature Structural Biology, Vol. 9, p. 268 (2002) reported inhibitors p38 alpha-kinase also disclosed in WO Publication No. 00/43384 and Regan et al., J. Medicinal Chemistry, Vol. 45, pp. 2994-3008 (2002). This class of inhibitors also interacts with the kinase at the ATP active site involving a concomitant movement of the kinase activation loop.

More recently, it has been disclosed that kinases utilize activation loops and kinase domain regulatory pockets to control their state of catalytic activity. This has been recently reviewed: see M. Huse and J. Kuriyan, Cell (2002) 109:275.

### SUMMARY OF THE INVENTION

The present invention is directed to methods of identifying molecules which interact with specific naturally occurring proteins (e.g., mammalian, and especially human proteins) in order to modulate the activity of the proteins, as well as novel protein-small molecule modulator adducts. In its method aspects, the invention exploits a characteristic of naturally occurring proteins, namely that the proteins change their conformations in vivo with a corresponding alteration in protein activity. For example, a given protein in one conformation may be biologically upregulated as an enzyme, while in another conformation, the same protein may be biologically downregulated. Moreover, the invention preferably makes use of one mechanism of conformation change utilized by naturally occurring proteins, through the interaction of what are termed “switch control ligands” and “switch control pockets” within the protein.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 is a schematic representation of a naturally occurring mammalian protein in accordance with the invention including “on” and “off” switch control pockets, a transiently modifiable switch control ligand, and an active ATP site;

FIG. 2 is a schematic representation of the protein of FIG. 1, wherein the switch control ligand is illustrated in a binding relationship with the off switch control pocket, thereby causing the protein to assume a first biologically downregulated conformation;

FIG. 3 is a view similar to that of FIG. 1, but illustrating the switch control ligand in its charged-modified condition wherein the OH groups of certain amino acid residues have been phosphorylated;

FIG. 4 is a view similar to that of FIG. 2, but depicting the protein wherein the switch control ligand is in a binding relationship with the on switch control pocket, thereby causing the protein to assume a second biologically-active conformation different than the first conformation of FIG. 2;

FIG. 4a is an enlarged schematic view illustrating a representative binding between the phosphorylated residues of the switch control ligand, and complemental residues from the on switch control pocket;

FIG. 5 is a view similar to that of FIG. 1, but illustrating in schematic form possible small molecule compounds in a binding relationship with the on and off switch control pockets;

FIG. 6 is a schematic view of the protein in a situation where a composite switch control pocket is formed with portions of the switch control ligand and the on switch control pocket, and with a small molecule in binding relationship with the composite pocket;

FIG. 7 is a schematic view of the protein in a situation where a combined switch control pocket is formed with portions of the on switch control pocket, the switch control ligand sequence, and the active ATP site, and with a small molecule in binding relationship with the combined switch control pocket;

FIG. 8 is a X-ray crystal structural ribbon diagram illustrating the on conformation of the insulin receptor kinase protein in its biologically upregulated state;

FIG. 9 is a similar to FIG. 8 but depicts the protein in the off conformation in its biologically downregulated state;

FIG. 10 is a SURFNET visualization of abl kinase, with the on switch control pocket illustrated in blue;

FIG. 11 is a GRASP visualization of abl kinase, with the on switch control pocket encircled in yellow;

FIG. 12 is ribbon diagram of the abl kinase protein, with important amino acid residues of the on switch control pocket identified;

FIG. 13 is a ribbon diagram of the abl kinase protein illustrating the combined switch control pocket (on switch control pocket/switch control ligand sequence/ATP active site);

FIG. 14 is a SURFNET visualization of p38 kinase with the on switch control pocket illustrated in blue;

FIG. 15 is a GRASP visualization of p38 kinase with the on switch control pocket encircled in yellow;

FIG. 16 is a ribbon diagram of p38 kinase protein with important amino acid residues of the on switch control pocket identified;

FIG. 17 is a SURFNET visualization of Gsk-3 beta kinase protein with the dual functionality on-off switch control pocket illustrated in blue;

FIG. 18 is a GRASP visualization of Gsk-3 beta kinase protein with the dual functionality on-off switch control pocket encircled in yellow;

FIG. 19 is ribbon diagram of Gsk-3 beta kinase protein with important amino acid residues of the combination on-off switch control pocket identified;

FIG. 20 is a SDS-PAGE gel identifying the semi-purified abl kinase domain protein in its unphosphorylated state;

FIG. 21 is a SDS-PAGE gel identifying the purified abl kinase protein in its unphosphorylated state;

FIG. 22 is the chromatogram elution profile of semi-purified abl kinase domain protein;

FIG. 23 is the chromatogram elution profile of purified abl kinase domain protein;

FIG. 24 is an SDS-PAGE gel identifying abl kinase protein before (lanes 2-4) and after (lanes 5-8) and after TEV tag cleavage;

FIG. 25 is a UV spectrum of purified abl protein with the small molecule inhibitor PD 180790 bound to the ATP site of the protein;

FIG. 26 is the chromatogram elution profile of abl construct 5 protein (abl 1-531, Y412F mutant) upon purification through Nickel affinity chromatography and Q-Sepharose chromatography;

FIG. 27 is SDS-PAGE gel of purified abl construct 5 protein;

FIG. 28 is the chromatogram elution profile of purified p38-alpha kinase protein in its unphosphorylated state;

FIG. 29 is SDS-PAGE gel of purified p38-alpha kinase protein in its unphosphorylated state;

FIG. 30 is a mass spectrogram of activated Gsk3-beta protein in its phosphorylated state;

FIG. 31 is a mass spectrogram of unactivated Gsk3-beta protein in its unphosphorylated state;

FIG. 32 is a Western Blot analysis staining of phosphorylated Gsk3-beta protein with the anti-phosphotyrosine antibody; and

FIG. 33 is a Western Blot analysis staining of unphosphorylated Gsk3-beta protein with the anti-phosphotyrosine antibody.

### DETAILED DESCRIPTION OF THE PREFERRED EMBODIMENTS

The present invention provides a way of rationally developing new small molecule modulators which interact with naturally occurring proteins (e.g., mammalian, and especially human proteins) in order to modulate the activity of the proteins. Novel protein-small molecule adducts are also provided. The invention preferably makes use of naturally occurring proteins having a conformational property whereby the proteins change their conformations in vivo with a corresponding change in protein activity. For example, a given enzyme protein in one conformation may be biologically upregulated, while in another conformation, the same protein may be biologically downregulated. The invention preferably makes use of one mechanism of conformation change utilized by naturally occurring proteins, through the interaction of what are termed “switch control ligands” and “switch control pockets” within the protein.

As used herein, “switch control ligand” means a region or domain within a naturally occurring protein and having one or more amino acid residues therein which are transiently modified in vivo between individual states by biochemical modification, typically phosphorylation, sulfation, acylation or oxidation. Similarly, “switch control pocket” means a plurality of contiguous or non-contiguous amino acid residues within a naturally occurring protein and comprising residues capable of binding in vivo with transiently modified residues of a switch control ligand in one of the individual states thereof in order to induce or restrict the conformation of the protein and thereby modulate the biological activity of the protein, and/or which is capable of binding with a non-naturally occurring switch control modulator molecule to induce or restrict a protein conformation and thereby modulate the biological activity of the protein.

A protein-modulator adduct in accordance with the invention comprises a naturally occurring protein having a switch control pocket with a non-naturally occurring molecule bound to the protein at the region of said switch control pocket, said molecule serving to at least partially regulate the biological activity of said protein by inducing or restricting the conformation of the protein. Preferably, the protein also has a corresponding switch control ligand, the ligand interacting in vivo with the pocket to regulate the conformation and biological activity of the protein such that the protein will assume a first conformation and a first biological activity upon the ligand-pocket interaction, and will assume a second, different conformation and biological activity in the absence of the ligand-pocket interaction.

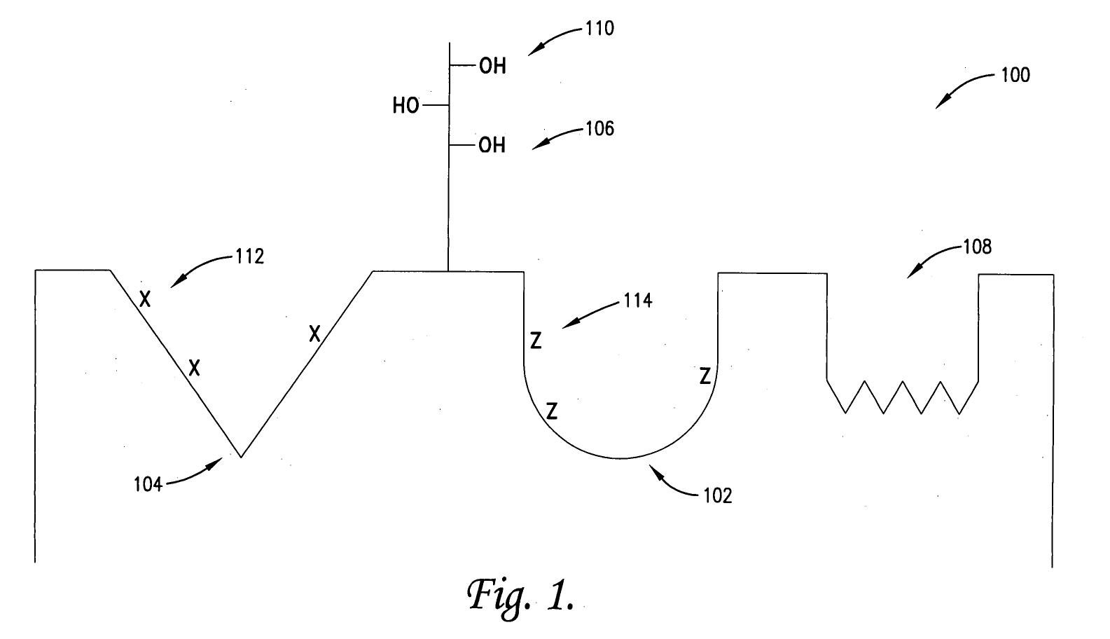

The nature of the switch control ligand/switch control pocket interaction may be understood from a consideration of schematic FIGS. 1-4. Specifically, in FIG. 1, a protein 100 is illustrated in schematic form to include an “on” switch control pocket 102, and “off” switch control pocket 104, and a switch control ligand 106. In addition, the schematically depicted protein also includes an ATP active site 108. In the exemplary protein of FIG. 1, the ligand 106 has three amino acid residues with side chain OH groups 110. The off pocket 104 contains corresponding X residues 112 and the on pocket 102 has Z residues 114. In the exemplary instance, the protein 100 will change its conformation depending upon the charge status of the OH groups 110 on ligand 106, i.e., when the OH groups are unmodified, a neutral charge is presented, but when these groups are phosphorylated a negative charge is presented.

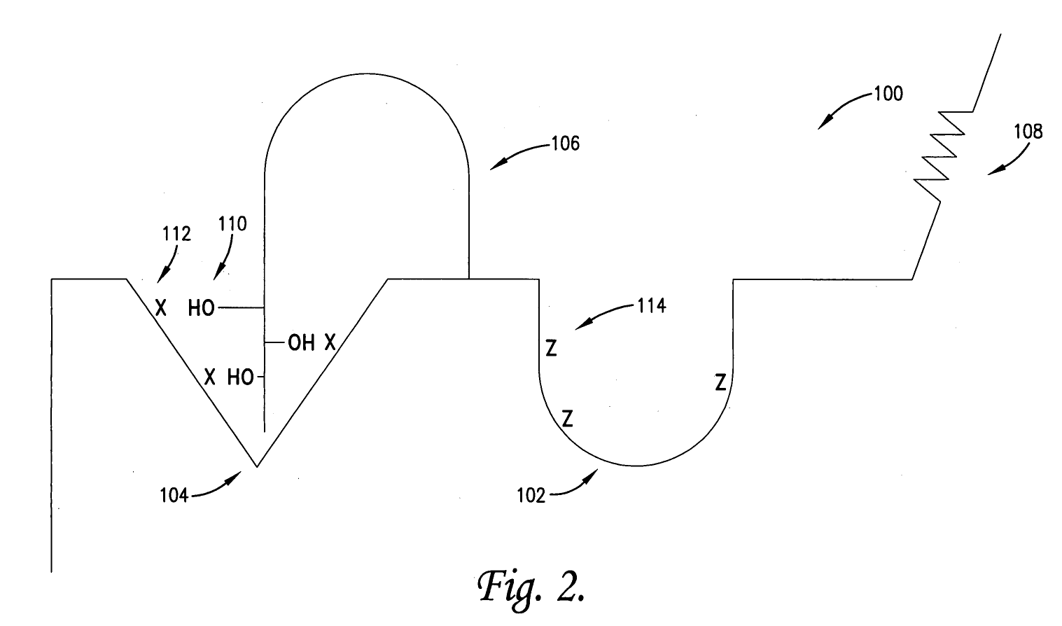

The functionality of the pockets 102, 104 and ligand 106 can be understood from a consideration of FIGS. 2-4. In FIG. 2, the ligand 106 is shown operatively interacted with the off pocket 104 such that the OH groups 110 interact with the X residues 112 forming a part of the pocket 104. Such interaction is primarily by virtue of hydrogen bonding between the OH groups 110 and the residues 112. As seen, this ligand/pocket interaction causes the protein 100 to assume a conformation different from that seen in FIG. 1 and corresponding to the off or biologically downregulated conformation of the protein.

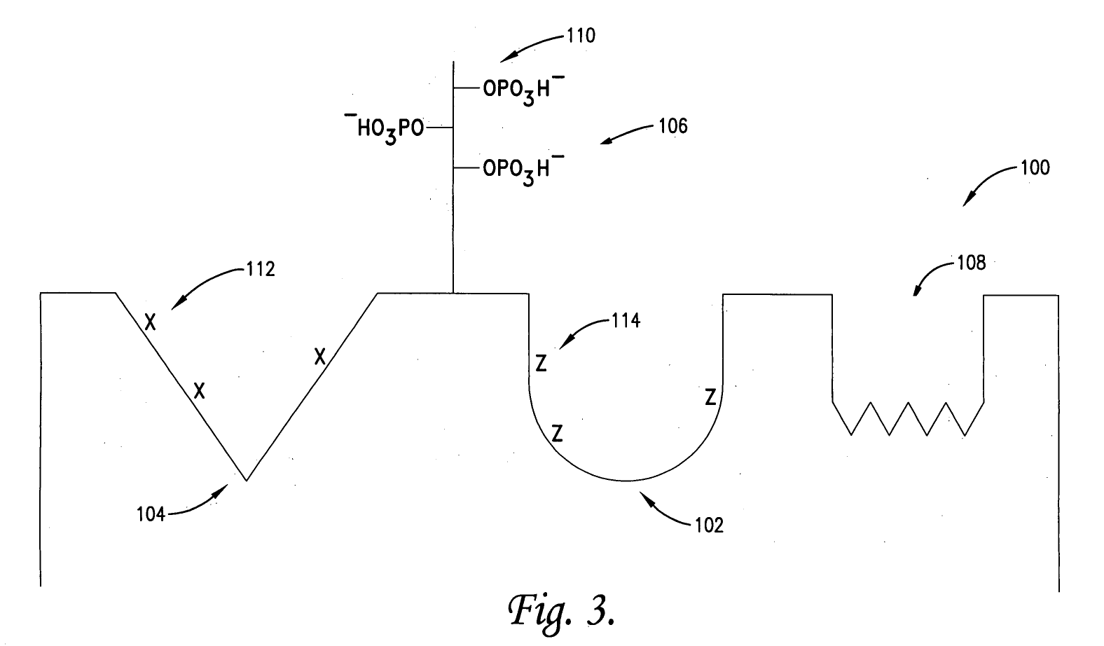

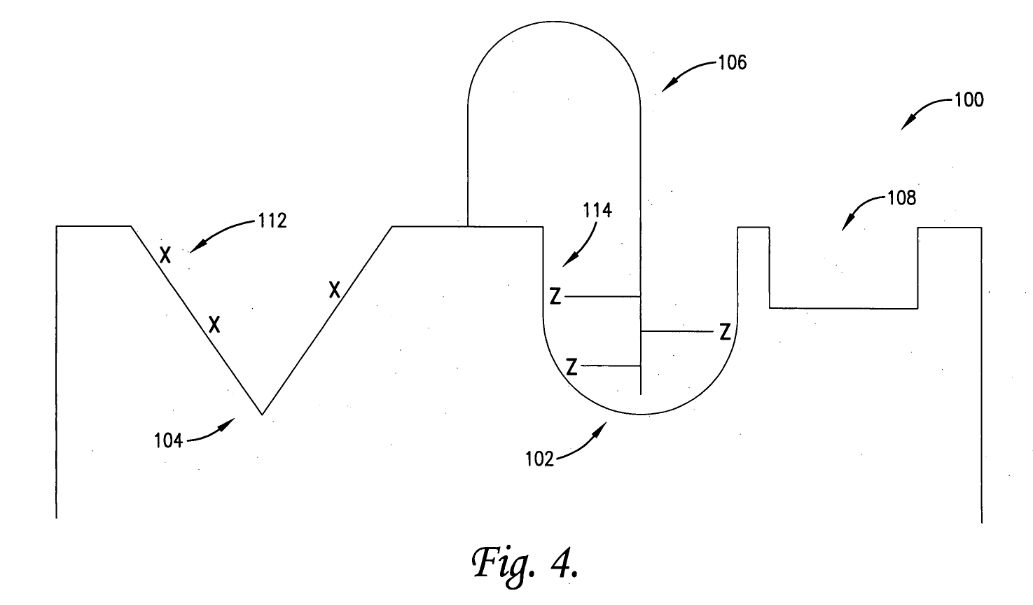

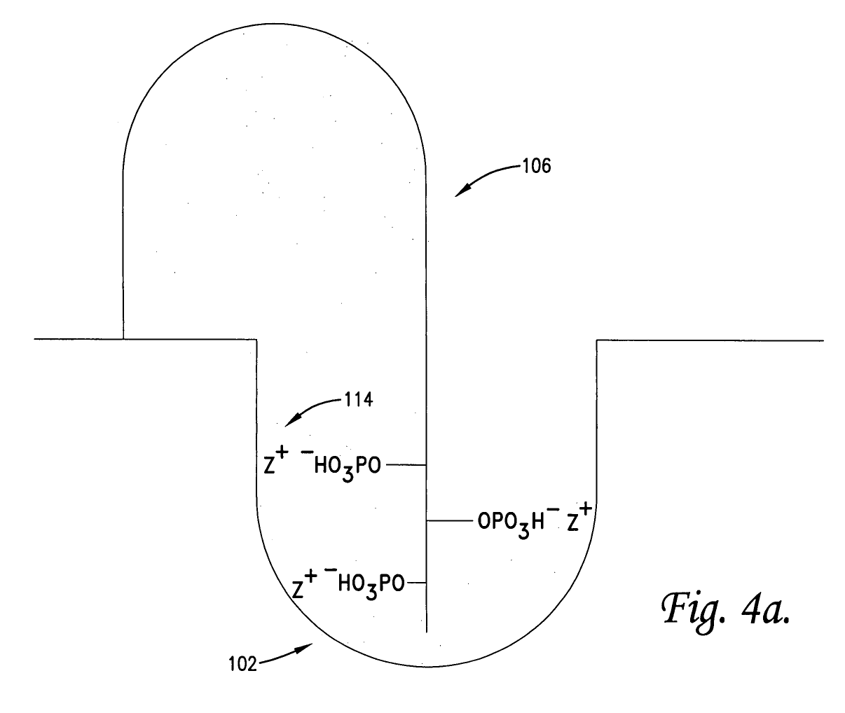

FIG. 3 illustrates the situation where the ligand 106 has shifted from the off pocket interaction conformation of FIG. 2 and the OH groups 110 have been phosphorylated, giving a negative charge to the ligand. In this condition, the ligand has a strong propensity to interact with on pocket 102, to thereby change the protein conformation to the on or biologically upregulated state (FIG. 4). FIG. 4a illustrates that the phosphorylated groups on the ligand 106 are attracted to positively charged residues 114 to achieve an ionic-like stabilizing bond. Note that in the on conformation of FIG. 4, the protein conformation is different than the off conformation of FIG. 2, and that the ATP active site is available and the protein is functional as a kinase enzyme.

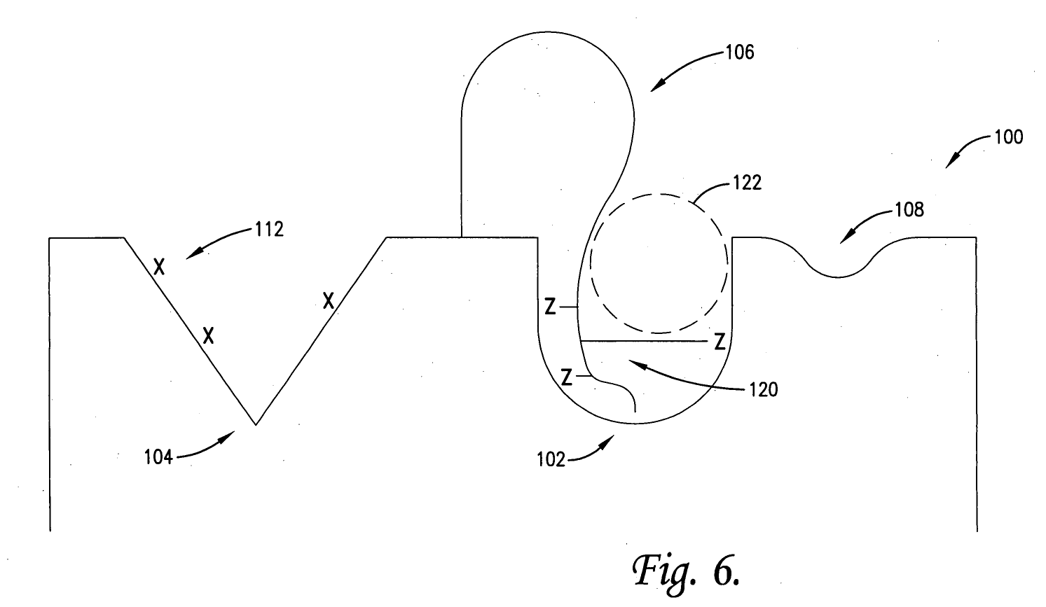

FIGS. 1-4 illustrate a simple situation where the protein exhibits discrete pockets 102 and 104 and ligand 106. However, in many cases a more complex switch control pocket pattern is observed. FIG. 6 illustrates a situation where an appropriate pocket for small molecule interaction is formed from amino acid residues taken both from ligand 106 and, for example, from pocket 102. This is termed a “composite switch control pocket” made up of residues from both the ligand 106 and a pocket, and is referred to by the numeral 120. A small molecule 122 is illustrated which interacts with the pocket 120 for protein modulation purposes.

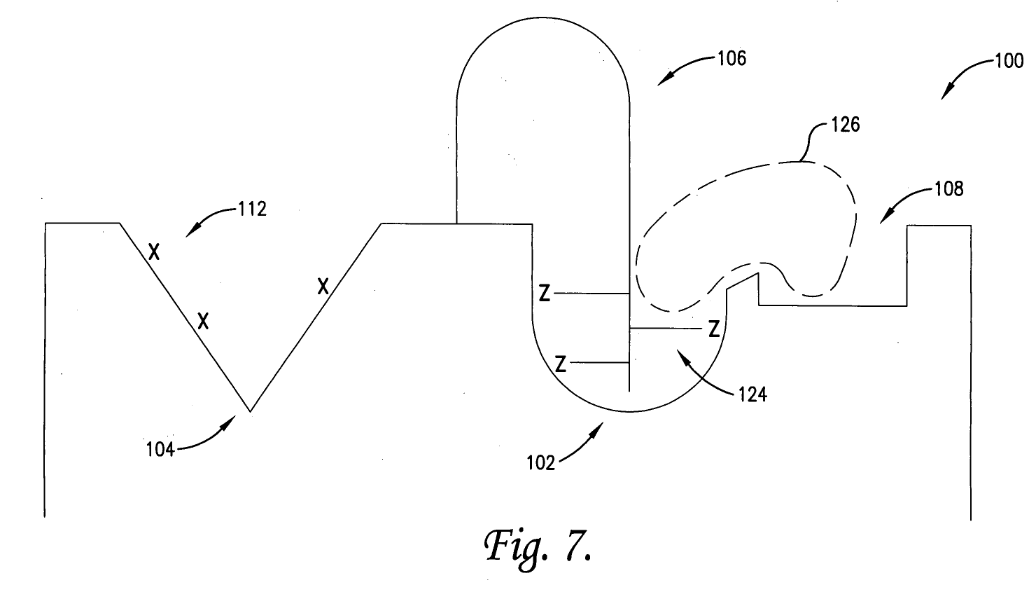

Another more complex switch pocket is depicted in FIG. 7 wherein the pocket includes residues from on pocket 102, and ATP site 108 to create what is termed a “combined switch control pocket.” Such a combined pocket is referred to as numeral 124 and may also include residues from ligand 106. An appropriate small molecule 126 is illustrated with pocket 124 for protein modulation purposes.

It will thus be appreciated that while in the simple pocket situation of FIGS. 1-4, the small molecule will interact with the simple pocket 102 or 104, in the more complex situations of FIGS. 6 and 7 the interactive pockets are in the regions of the pockets 120 or 124. Thus, broadly the the small molecules interact “at the region” of the respective switch control pocket.

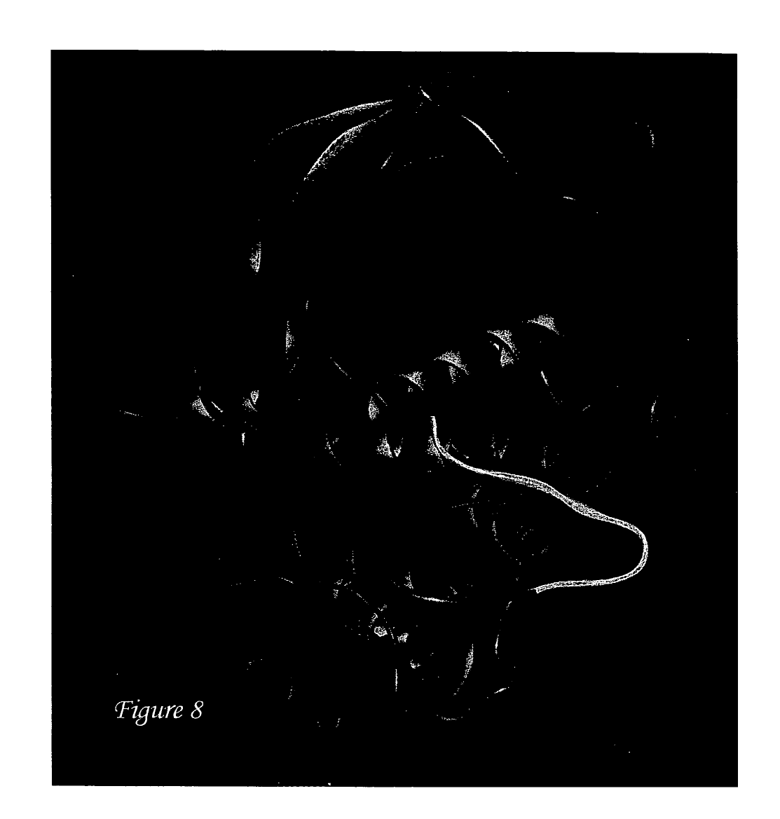

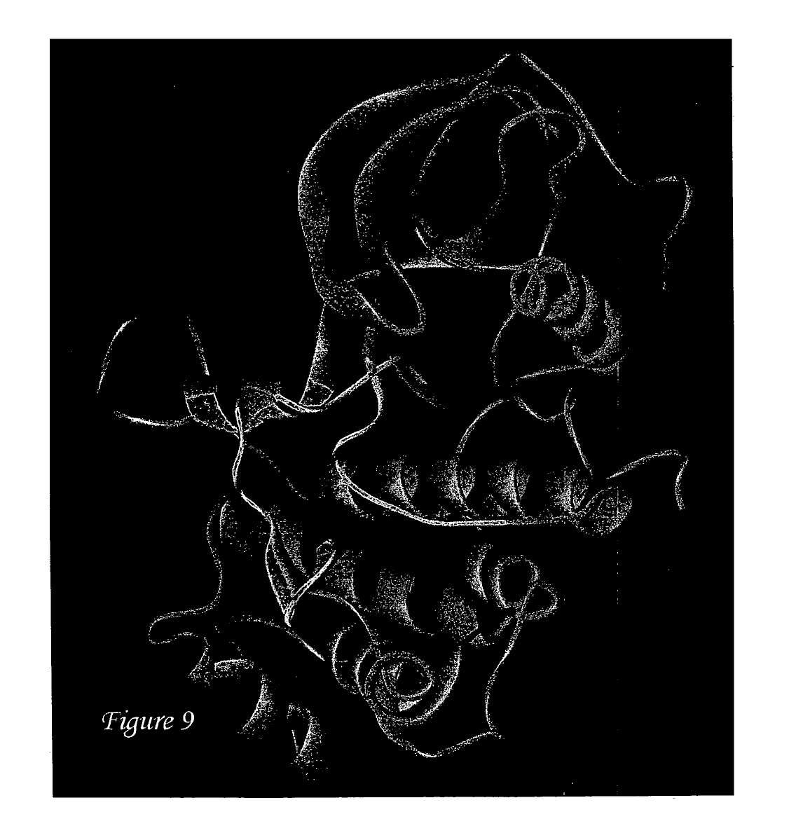

FIGS. 8 and 9 are ribbon diagrams derived from X-ray crystallography analysis of the insulin receptor kinase domain protein, where FIG. 8 illustrates the protein in its on or biologically upregulated conformation, shown in blue. In this photograph, the yellow-colored strand is the switch control ligand sequence, whereas the magenta portions represent key residues forming the complemental on-switch control pocket which interacts with the ligand sequence to maintain the protein in the biologically upregulated conformation. FIG. 9 on the other hand depicts the protein in its off or biologically downregulated conformation, shown in simulated brass color. In this diagram, the switch control sequence is again depicted in yellow and key residues of the off-switch control pocket are illustrated in green. Again, the interaction between the switch control ligand and the off-switch control pocket maintains the protein in the depicted biologically downregulated conformation.

Referring again to the schematic depictions, the FIG. 8 diagram corresponds to FIG. 4 wherein the ligand 106 interacts with on pocket 102. Likewise, FIG. 9 corresponds to FIG. 2 wherein ligand 106 interacts with pocket 104.

Those skilled in the art will appreciate that a given protein will “switch” over time between the upregulated and downregulated conformations based upon the phosphorylation of ligand 106 tending to shift the protein to the on pocket interaction, or cleaving of the phosphate groups from the ligand tending to shift the protein to the off pocket interaction conformation. Thus, the conformation change effected by the switch control ligand/switch control pocket interaction is dynamic in nature and is ultimately governed by intracellular conditions.

It will also be understood that abnormalities in protein conformation can lead to or exacerbate diseases. For example, if a given protein untowardly remains in the off or biologically downregulated conformation, metabolic processes requiring the active protein will be prevented, retarded or unwanted side reactions may occur. Similarly, if a protein untowardly remains in the on or biologically upregulated conformation, the metabolic process may be unduly promoted which can also result in serious health problems.

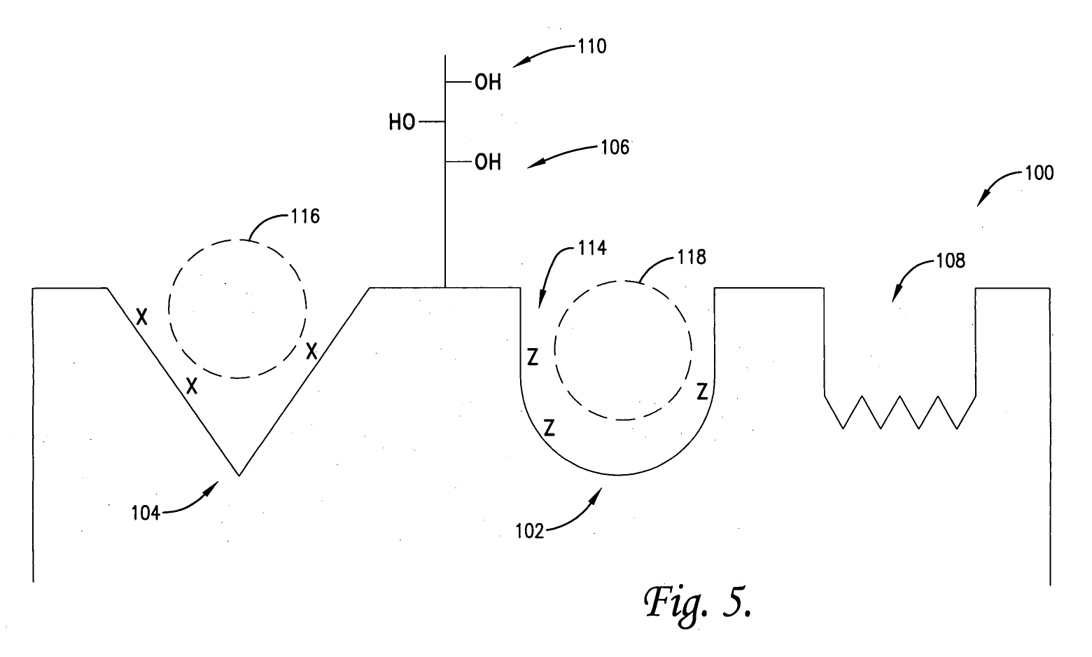

However, it has been found that small molecule compounds can be developed which will modulate protein activity so as to duplicate or approach normal in vivo protein activity. Referring to FIG. 5, it will be seen that a small molecule 116 may interact with off pocket 104 so as to inhibit ligand 106 from interacting with the pocket 104. In this simplified hypothetical, the protein 100 would then have a greater propensity to remain in the on or biologically upregulated conformation. As an alternative, a small molecule 118 is shown interacting with on pocket 102 so as to inhibit ligand 106 from interaction with the pocket 102. Under this simplified scheme, this would result in a greater propensity for the ligand 106 to interact with off pocket 104, thereby causing the protein to move to its off or biologically downregulated conformation.

Hence, analysis of proteins to ascertain the location and sequences of interacting switch control ligands and switch control pockets, together with an understanding of how these interact to switch the protein between biologically upregulated and downregulated conformations, provides a powerful tool which can be used in the design and development of small molecule compounds which can modulate protein activity.

Broadly speaking, the method of identifying molecules which interact with specific naturally occurring proteins in order to modulate protein activity involves first identifying a switch control ligand forming a part of the protein, and a switch control pocket also forming a part of the protein and which interacts with the ligand. The ligand and pocket cooperatively interact to regulate the conformation and biological activity of the protein, such that the protein will assume a first conformation and a corresponding first biological activity upon the ligand-pocket interaction, and will assume a second, different conformation and biological activity in the absence of the ligand-pocket interaction.

In the next step, respective samples of the protein in the first and second conformations thereof are provided, and these protein samples are used in screening assays of candidate small molecules. Such screening broadly involves contacting the candidate molecules with at least one of the samples, and identifying which of the small molecules bind with the protein at the region of the identified switch control pocket.

The method of the invention is applicable to a wide variety of naturally occurring mammalian (e.g., human) proteins, which may be wild type consensus proteins, disease polymorphs, disease fusion proteins and/or artificially engineered variant proteins. Classes of applicable proteins would include enzymes, receptors, and signaling proteins; more particularly, the kinases, phosphotases, sulfotranferases, sulfatases, transcription factors, nuclear hormone receptors, g-protein coupled receptors, g-proteins, gtp-ases, hormones, polymerases, and other proteins containing nucleotide regulatory sites. In most instances, proteins of interest would have a molecular weight of at least 15 kDa, and more usually above about 30 kDa.

In the course of the method of the invention, a number of techniques may be used to identify switch control ligand sequence(s) and switch control pocket(s) and to determine the upregulation or downregulation effects of candidate small molecule modulators. Broadly speaking, these methods comprise analysis of bioinformatics, X-ray crystallography, nuclear magnetic resonance spectroscopy (NMR), circular dichroism (CD), and affinity base screening. In addition, entirely conventional techniques such as site directed mutagenesis and standard biochemical experiments may also be of assistance.

Bioinformatic analysis permits identification of relevant ligands and pockets without the need for experimentation. For example, relevant protein data can be in some cases determined strictly through use of available databases such as PUBMED. Thus, an initial step may be a PUBMED inquiry regarding known structures of a protein of interest, which contains sequence information. Next, BLAST searches may be conducted, in order to ascertain other sequences containing a selected minimum stringency (e.g., at least 60%). This may reveal point mutations or polymorphisms of interest, as well as abnormal fusion proteins, all of which may be causative of disease; these may also provide insights into the identification of functional or dysfunctional switch control ligand sequences and/or pockets causative of disease. A specific example of such bioinformatic analysis is set forth in Example 1 below.

X-ray crystallography techniques first require protein expression affording highly purified proteins. Whole gene synthesis technology may be used to chemically synthesize protein genes optimized for the particular expression systems used. Conventional technology can be employed to rapidly synthesize any gene from synthetic oligonucleotides. Software (Gene Builder™) and associated molecular biology methods allow any gene to be synthesized. Whole gene synthesis is advantageous over traditional cloning methods because the codon optimized version of the gene can be rapidly synthesized for optimal expression. In addition, complex mutations (e.g. combining many different mutations) can be made in one step instead of sequentially. Strategic placement of restriction sites facilitates the rapid addition additional mutations as needed. This technology therefore allows many more gene constructs to be created in a shorter amount of time. Protein sequence selection is determined using a combination of phylogenetic analyses, molecular modeling and structural predictions, known expression, functional screening data, and reported literature data to develop a strategy for protein production. Expression constructs can be made using commercially available and/or vectors to express the proteins in baculovirus-infected insect cells. E. coli expression systems may be used for production of other proteins. The genes may be modified by adding affinity tags. The genes may also be modified by creating deletions, point mutations, and protein fusions to improve expression, aid purification and facilitate crystallization.

Protein Purification: Total cell paste from expression experiments may be disrupted by nitrogen cavitation, French press, or microfluidization which ever proves to be the most effective for releasing soluble protein. The extracts are subjected to parallel protein purification using the a robotic device that simultaneously runs multiple columns (including Glu-mAb, metal chelate, Q-seph, S-Seph, Phenyl-Seph, and Cibacron Blue) in parallel under standard procedures and the fractions are analyzed by SDS-PAGE. This information is combined with the published purification protocols to rapidly develop purification protocols. Once purified, the protein is subjected to a number of biophysical assays (Dynamic Light Scattering, UV absorption, MALDI-ToF, analytical gel filtration etc.).

Crystal Growth and X-ray Diffraction Quality Analysis: Sparse matrix and focused crystallization screens are set up with and without ligands at 2 or more temperatures. Crystals obtained without ligands (apo-crystals) are used for ligand soaking experiments. Crystal growth conditions are optimized for protein-crystals based on initial results. Once suitable protein-crystals have been obtained, they are screened to determine their diffraction quality under various cryo-preservation conditions on an R-AXIS IV imaging plate system and an X-STREAM cryostat. Protein-crystals of sufficient diffraction quality are used for X-ray diffraction data collection, or are stored in liquid nitrogen and saved for subsequent data collection at a synchrotron X-ray radiation source. The diffraction limits of protein-crystals are determined by taking at least two diffraction images at phi spindle settings 90° apart. The phi spindle is oscillated 1° during diffraction image collection. Both images are processed by the HKL-2000 suite of X-ray data analysis and reduction software. The diffraction resolution of the protein-crystals are accepted as the higher resolution limit of the resolution shell in which 50% or more of the indexed reflections have an intensity of 1 sigma or greater.

X-ray Diffraction Data Collection: If the protein-crystals are found to diffract to 3.0 Å or better on the R-AXIS IV system or at a synchrotron, then a complete data set are collected at a synchrotron. A complete data set is defined as having at least 90% of all reflections in the highest resolution shell have been collected. The X-ray diffraction data are processed (reduced to unique reflections and intensities) using the HKL-2000 suite of X-ray diffraction data processing software.

Structure Determination: The structures of the proteins are determined by molecular replacement (MR) using one or more protein search models. This MR method uses the protein coordinate sets available in the Protein Data Bank (PDB). If necessary, the structure determination is facilitated by multiple isomorphous replacement (MIR) with heavy atoms and/or multi-wavelength anomalous diffraction (MAD) methods. MAD synchrotron data sets are collected for heavy atom soaked crystals if EXAFS scans of the crystals (after having been washed in mother liquor or cryoprotectant without heavy atom) reveal the appropriate heavy atom signal. Analysis of the heavy atom data sets for derivatization is completed using the CCP4 crystallographic suite of computational programs. Heavy atom sites are identified by (|F_{PH}|−|F_{P}|)² difference Patterson and the (|F⁺|−|F⁻|)² anomalous difference Patterson map.

High field nuclear magnetic resonance (NMR) spectroscopic methods can also be utilized to identify switch control ligand sequences and pockets. NMR studies have been reported to elucidate the structures of proteins and in particular protein kinases. (Wuthrich, K; “NMR of Proteins and Nucleic Acids” Wiley-Interscience: 1986; Evans, J. N. S., Biomolecular Nmr Spectroscopy, Oxford University Press: 1995; Cavanagh, J.; et al., N. Protein Nmr Spectroscopy: Principals and Practice, Academic Press: 1996.; Krishna, N. R.; Berliner, L. J. Protein Nmr for the Millenium (Biological Magnetic Resonance, 20), Plenum Pub Corp: 2003.

Over the last 20 years, NMR has evolved into a powerful technique to probe protein structures, the interaction of proteins with other biomolecules and expose the details of small-molecule-protein interactions. NMR methods are complementary to X-ray crystallographic methods, and the combination of the two techniques provides a powerful strategy to reveal the nature of protein/small molecule interactions. A particularly advantageous NMR technique involves the preparation of ¹⁵N and/or ¹³C labeled protein and analyzing chemical shift perturbations which occur upon conformational changes of the protein effected by interaction of the protein's switch control ligand sequence with its respective switch control pocket or interaction of a small molecule modulator with a switch control pocket region.

Circular dichroism (CD) is a technique suited for the study of protein conformation (Johnson, W. C., Jr.; Circular Dichroism Spectroscopy and the vacuum ultraviolet region; Ann. Rev. Phys. Chem. (1978) 29:93; Johnson, W. C., Jr.; Protein secondary structure and circular dichroism: A practical guide” Proteins: Str. Func. Gen. (1990) 7:205; Woody, R. W. “Circular dichroism of peptides” (Chapter 2) The Peptides Volume 7 1985 Academic Press; Berova, N., Nakanishi, K., Woody, R. W., Circular Dichroism: Principles and Applications, 2nd Ed. Wiley-VCH, New York, 2000; Schmid, F. X.; Spectral methods of characterizing protein conformation and conformational changes in Protein Structure, a practical approach edited by T. E. Creighton, IRL Press, Oxford 1989) and in particular has been reported for the study of protein kinase conformation changes. (Bosca, L.; Moran, F.; Circular dichroism analysis of ligand-induced conformational changes in protein kinase C. Mechanism of translocation of the enzyme from the cytosol to the membranes and its implications. Biochemical J. (1993) 290:827; Okishio, N.; Tanaka, T.; Fukuda, R.; Nagai, M.; Differential Ligand Recognition by the Src and Phosphatidylinositol 3-Kinase Src Homology 3 Domains: Circular Dichroism and Ultraviolet Resonance Raman Studies; Biochemistry (2003) 42: 208; Deng, Z.; Roberts, D.; Wang, X.; Kemp, R. G.; Expression, characterization, and crystallization of the pyrophosphate-dependent phosphofructo-1-kinase of Borrelia burgdorferi. Arch. Biochem. Biophys. (1999) 371: 326; Reed, J; Kinzel, V; Kemp, B. E.; Cheng, H. C.; Walsh, D. A.; Circular dichroic evidence for an ordered sequence of ligand/binding site interactions in the catalytic reaction of the cAMP-dependent protein kinase. Biochemistry (1985) 24: 2967; Okishio, N.; Tanaka, T.; Nagai, M.; Fukuda, R.; Nagatomo, S.; Kitagawa, T.; Identification of Tyrosine Residues Involved in Ligand Recognition by the Phosphatidylinositol 3-Kinase Src Homology 3 Domain: Circular Dichroism and UV Resonance Raman Studies., Biochemistry (2001) 40: 15797; Okishio, N.; Tanaka, T.; Fukuda, R.; Nagai, M.; Role of the Conserved Acidic Residue Asp21 in the Structure of Phosphatidylinositol 3-Kinase Src Homology 3 Domain: Circular Dichroism and Nuclear Magnetic Resonance Studies, Biochemistry (2001) 40: 119; Mattsson, P. T.; Lappalainen, I.; Backesjo, C. -M.; Brockmann, E.; Lauren, S.; Vihinen, M.; Smith, C. I. E.; “Six X-linked agammaglobulinemia-causing missense mutations in the Src homology 2 domain of Bruton's tyrosine kinase: phosphotyrosine-binding and circular dichroism analysis.” J. Immun. (2000) 164: 4170; Raimbault, C.; Couthon, F.; Vial, C.; Buchet, R.; “Effects of pH and KCl on the conformations of creatine kinase from rabbit muscle. Infrared, circular dichroic, and fluorescence studies.” Euro. J. Biochem. (1995) 234: 570; Shah, J.; Shipley, G. G.; Circular dichroic studies of protein kinase C and its interactions with calcium and lipid vesicles. Biochim. Biophys. Acta (1992) 1119: 19).

The more pronounced helical organization and conformational movements that occur upon kinase activation (upregulation) compared to downregulation states can be observed by CD. Switch control pocket-based small molecule modulation can result in stabilization of a predominant conformational state. Correlation of CD spectra obtained in the presence of small molecular modulators with those obtained in the absence of modulators allows the determination of the nature of small-molecule binding and differentiate such binding from that of conventional ATP-competitive inhibitors.

A variety of bio-analytical methods can provide small molecule binding affinities to proteins. Affinity-based screening methods using capillary zone electrophoresis (CZE) may be employed in the early stages of screening of candidate small molecule modulators. Direct determination of Kds (disassociation constants) of the small molecule modulator-protein interactions can be obtained. (Heegaard, N. H. H.; Nilsson, S.; Guzman, N. A.; Affinity capillary electrophoresis: important application areas and some recent developments; J. Chromatography B (1998)715: 29-54; Yen-Ho Chu, Y. -H.; Lees, W. J.; Stassinopoulos, A.; Walsh, C. T.; Using Affinity Capillary Electrophoresis To Determine Binding Stoichiometries of Protein-Ligand Interactions, Biochemistry (1994) 33: 10616-10621; Davis, R. G.; Anderegg R. J.; Blanchard, S. G., Iterative size-exclusion chromatography coupled with liquid chromatographic mass spectrometry to enrich and identify tight-binding ligands from complex mixtures, Tetrahedron (1999) 55: 11653-1166; Shen Hu, S.; Dovichi, N. J.; Capillary Electrophoresis for the Analysis of Biopolymers; Anal. Chem. (2002) 74: 2833-2850; Honda, S.; Taga, A.; Suzuki, K.; Suzuki, S.; Kakhi, K., Determination of the association constant of monovalent mode protein-sugar interaction by capillary zone eletrophoresis, J. Chromatography B (1992) 597: 377-382; Colton, I. J.; Carbeck, J. D.; Rao, J.; Whitesides, G. M., Affinity Capillary Electrophoresis: A physical-organic tool for studying interaction in biomolecular recognition, Electrophoresis (1998) 19: 367-382.

Another affinity based screening method makes use of reporter fluoroprobe binding to a candidate protein. Candidate small molecule modulators are screened in this fluoroprobe assay. Compounds which do bind to the protein are measured by a decrease in the fluorescence of the fluoroprobe reporter. This method is reported in the following Example 1.

The invention also pertains to small molecule modulator-protein adducts. The proteins are of the type defined previously. Insofar as the modulators are concerned, they should have functional groups complemental with active residues within the switch control pocket regions, in order to maximize modulator-protein binding. For example, in the case of the kinases, it has been found that modulators having 1-3 dicarbonyl linkages are often useful. Where switch control pockets of cationic character are found, the small molecule modulators would often have acidic functional groups or moieties, e.g., sulfonic, phosphonic, or carboxylic groups. In terms of molecular weight, preferred modulators would typically have a molecular weight of from about 120-650 Da, and more preferably from about 300-550 Da. If these small molecule modulators are to be studied in whole cell environments, their properties should conform to well understood principles that optimize the small molecule modulators for cell penetrability (Lipinski's Rule of 5, Advanced Drug Delivery Reviews, Vol. 23, Issues 1-3, pp 3-25 (1997)).

The invention also provides methods of altering the biological activity of proteins broadly comprising the steps of first providing a naturally occurring protein having a switch control pocket. Such a protein is then contacted with a non-naturally occurring molecule modulator under conditions to cause the modulator to bind with the protein at the region of the pocket in order to at least partially regulate the biological activity of the protein by inducing or restricting the conformation of the protein.

The following examples set forth representative methods in accordance with the invention. It is to be understood, however, that these examples are provided by way of illustration and nothing therein should be taken as a limitation upon the overall scope of the invention.

### EXAMPLE 1

In this example, techniques are illustrated for the identification and/or development of small molecules which will interact at the region of switch control pockets forming a part of naturally occurring proteins, in order to modulate the in vivo biological activity of the proteins. Specifically, a family of known kinase proteins are analyzed using the process of the invention, namely the abl, p38-alpha, Gsk-3beta, insulin receptor-1, protein kinase B/Akt and transforming growth factor B-I receptor kinases.

Step 1: Identification and Classification of Switch Control Ligands Within the Kinase Proteins

In general, the switch control ligands of the kinases can be identified from using sequence and structural data from the respective kinases, if sufficiently detailed information of this character is available. Thus, this step of the method can be accomplished without experimentation. The known data relative to the kinases permits ready identification of transiently modifiable amino acid residues, which in the case of these proteins are modified by phosphorylation or acylation. The probable extent of the entire switch control ligand sequence can then be deduced. An additional helpful factor in the case of the kinases is that the ligand often begins with a DFG sequence of residues (the single letter amino acid code is used throughout).

Abl Kinase

The full length BCR-Abl sequence is provided herein as SEQ ID NO. 34. One switch control ligand sequence of abl kinase and bcr-abl fusion protein kinase are constituted by the sequence: D381, F382, G383, L384, S385, R386, L387, M388, T389, G390, D391, T392, Y393, T394, A395, H396 (ligand 1) (SEQ ID NO. 1). Y393 becomes phosphorylated upon (bcr)abl activation by upstream regulatory kinases or by autophosphorylation, and thus is a transiently modified residue (Tanis et al, Moleulcar and Cellular Biology (2003) 23: 3884; Brasher and Van Etten, The Journal of Biological Chemistry (2000) 275: 35631). The switch control ligand sequence begins with DFG and terminates with H396.

An alternate switch control ligand has the sequence Myr-G2Q3Q4P5G6K7V8L9G10D11Q12R13R14P15S16L17 (ligand 2) (SEQ ID NO. 2). Ligand 2, specific to the abl kinase isoform 1B, is the N-terminal cap of the abl protein sequence, and in particular the N-terminal myristolyl group located on G2 (Glycine 2) (Jackson and Baltimore, (1989) EMBO Journal 8:449; Resh, Biochem Biophys. Acta (1999) 1451:1).

p38-alpha Kinase

The switch control ligand sequence of p38-alpha kinase (SEQ ID NO. 3) is constituted by the sequence: D168, F169, G170, L171, A172, R173, H174, T175, D176, D177, E178, M179, T180, G181, Y182, V183, A184, T185, R186, W187, Y188, R189(SEQ ID NO. 4). T180 and Y182 become phosphorylated upon p38-alpha activation by upstream regulatory kinases (see Wilson et al, Chemistry & Biology (1997) 4:423 and references therein), and thus are transiently modifiable residues.

Gsk-3 Beta Kinase

The full length Gsk-3 beta kinase sequence is provided herein as SEQ ID No. 32. The Gsk-3 beta kinase sequence corresponding to the 1GNG crystal structure is provided herein se SEQ ID NO. 33. The switch control ligand sequence of Gsk-3 beta kinase protein is constituted by the sequence: D200, F201, G202, S203, A204, K205, Q206, L207, V208, K209, G210, E211, P212, N213, V214, S215, Y216, I217, C218, S219, K220 (Gsk ligand 1) (SEQ ID NO. 5); Y216 becomes phosphorylated upon activation by upstream regulatory kinases (Hughes et al, EMBO Journal (1993) 12: 803; Lesort et al, Journal of Neurochemistry (1999) 72:576; ter Haar et al, Nature Structural Biology (2001) 8: 593 and references therein.

An alternative switch control ligand sequence is: G3, R4, P5, R6, T7, T8, S9, F10, A11, E12 (Gsk ligand 2) (SEQ ID NO. 6); S9 becomes phosphorylated by the action of the upstream kinase PKB/Akt (Dajani et al, Cell (2001) 105: 721) Cross et al, Nature (1995) 378:785). S9 is the transiently modifiable residue.

Insulin Receptor Kinase-1

The full length IRK-1 gene is provided herein as SEQ ID NO. 35. The sequence corresponding to the 1GAG crystal structure is provided herein as SEQ ID NO. 36. It is noted that at least the first residue is different in SEQ ID NO. 36 than in SEQ ID NO. 35. The control switch ligand sequence of insulin receptor kinase-1 is constituted by the sequence: D1150, F1151, G1152, M1153, T1154, R1155, D1156, I1157, Y1158, E1159, T1160, D1161, Y1162, Y1163, R1164, K1165, G1166, G1167, K1168, G1169, L1170(SEQ ID NO. 7). Y1158, Y1162, and Y1163 are the transiently modifiable residues and become phosphorylated upon activation of the insulin receptor by insulin (see Hubbard et al, EMBO Journal (1997) 16: 5572 and references therein).

Protein Kinase B/Atk

The full length Atk1 sequence is provided herein as SEQ ID NO. 37. The protein kinase B/Akt kinase-only domain is provided herein as SEQ ID NO. 38. It is noted that these sequences differ at the N and C terminii. Additionally, the kinase-only domain begins at residue 143 of the full length sequence. The switch control ligand sequence of protein kinase B/Atk is constituted by P468, H469, F470, P471, Q472, F473, S474, Y475, S476, A477, S478 (SEQ ID NO. 8). S474 is the transiently modifiable residue which is phosphorylated upon activation by upstream kinase regulatory proteins, thereby increasing PKB/Ptk activity 1,000 fold above unphosphorylated PKB/Atk (Yang et al, Molecular Cell (2002) 9:1227 and references therein).

Transforming Growth Factor B-I Receptor Kinase

The full length sequence of the TGF-B-I receptor kinase is provided herein as SEQ ID NO. 39. The switch control ligand of transforming growth factor B-I receptor kinase is T185, T186, S187, G188, S189, G190, S191, G192, L193, P194, L185, L196(SEQ ID NO. 9). T185, T186, S187, S189, and S191 are the transiently modifiable residues and are partially or fully phosphorylated upon activation by the kinase activity of Transforming Growth Factor B-II receptor (Wrana et al, Nature (1994) 370: 341; Chen and Weinberg, Proc. Natl. Acad. Sci. USA (1995) 92: 1565).

Step 2: Identification and Classification of Switch Control Pockets

As in the case of identification of the switch control ligands, the complemental switch control pockets may be deduced from published kinase data, and particularly by X-ray crystallography structural analysis. An initial step in this analysis was the identification of residues which would bind with the previously identified transiently modifiable residues within the corresponding switch control ligands.

Abl Kinase

X-ray crystal structural analysis of abl kinase (SEQ ID NO. 30) revealed a probable switch control pocket sequence based on structure 1FPU (SEQ ID NO. 10) (Schlindler et al, Science (2000) 289: 1938) and 1IEP (SEQ ID NO. 11) (Nagar et al, Cancer Research (2002) 62: 4236). The switch control pocket sequence is complemental with the previously identified switch control ligand 1 sequence for this kinase and has a cluster of 2 basic amino acids taken from a combination of the alpha-C helix (residues 279-293) and the catalytic loop (residues 359-368). Specifically, lysine 285 from the alpha-C helix and arginine 362 from the catalytic loop form a part of the switch control pocket, inasmuch as these residues stabilize the binding of the transiently modified (phosphorylated) residue Y393 from the switch control ligand. Other predicted amino acid residues which contribute to the switch control pocket include residues from the glycine rich loop (residues 253-279), the N-lobe (residue 271), the beta-5 strand (residues 313-318), other amino acids taken from the alpha-C helix (residues 280-290) and other amino acids taken from the catalytic loop (residues 359-368). Additionally a C-lobe residue 401 or 416 is predicted to form the base of this pocket.

Table 1 illustrates amino acids from the protein sequence which form the switch control pocket for ligand 1 of the (bcr)abl kinase. All references to amino acid residue position are relative to the full length protein and not to SEQ ID NO. 30 which begins at position 223 of the full length protein.

TABLE 1

| | | | | | | B-5 | | |
|---|---|---|---|---|---|---|---|---|
| | | | | | | beta | | |
| | | | | N-Lobe | | strand | | |
| Glycine | | | | | | | | |
| Rich | | | | | | | | |
| Loop | | | | | | | | |
| Y253 | D276 | E279 | | K271 | | I313 | T315 | E316 |
| | | | | M278 | | F317 | M318 | |
| alpha-C | | | | | | | | |
| Helix | | | | | | | | |
| V280 | E281 | E282 | F283 | L284 | K285 | E286 | A287 | A288 |
| V289 | M290 | | | | | | | |
| alpha-E | F359 | | | | | | | |
| Helix | | | | | | | | |
| Catalytic | | | | | | | | |
| Loop | | | | | | | | |
| F359 | I360 | H361 | R362 | D363 | N368 | | | |
| C-Lobe | | | | | | | | |
| F401 | F416 | | | | | | | |

X-ray crystal structural analysis of abl kinase revealed a probable switch control pocket sequence based on structure 1OPL (SEQ ID NOS. 12 and 13), which is complemental with ligand 2. Analysis of the X-ray crystal structure 1OPL of abl kinase isoform 1B reveals this probable switch control pocket (Nagar et al, Cell (2003) 112:859).

Table 2 illustrates amino acids from the protein sequence which form the switch control pocket complemental with ligand 2 of (bcr)abl kinase.

TABLE 2
SH2 Domain and C-Lobe Helical
Switch Control Pocket

| | | | | |
|---|---|---|---|---|
| alpha-A helix | | | | |
| S152 | R153 | N154 | E157 | Y158 |
| alpha-E Helix | | | | |
| A356 | L359 | L360 | Y361 | |
| N-Lobe Loop | | | | |
| N393 | | | | |
| alpha-F Helix | | | | |
| L448 | A452 | Y454 | | |
| alpha-H Helix | | | | |
| C483 | P484 | V487 | E481 | |
| alpha-I Helix | | | | |
| E513 | | | | |
| I-I' Loop | | | | |
| F516 | Q517 | | | |
| alpha-I' Helix | | | | |
| I521 | V525 | L529 | | |

p38-alpha Kinase

X-ray crystal structural analysis of p38-alpha kinase (SEQ ID NO. 31) reveals the probable switch control pocket based on structure 1KV2 (SEQ ID NO. 14) (Pargellis, et al.; Nat. Struct, Biol. 9 pp. 268-272 (2002). The switch control pocket for the previously identified switch control ligand sequence has a cluster of 2 basic amino acids taken from a combination of the alpha-C helix (residues 61-78) and the catalytic loop (residues 146-155). Specifically, arginine 67 and/or arginine 70 comes from the alpha-C helix, and arginine 149 comes from the catalytic loop. Other predicted amino acids which contribute to the switch control pocket include residues from the glycine rich loop (residues 34-36), amino acids taken from the alpha-C helix (residues 61-78), and amino acids taken from the catalytic loop (residues 146-155). Additionally amino acids taken from C-lobe residues 197-200 form the base of this pocket.

Table 3 illustrates amino acids from the protein sequence which form the switch control pocket.

TABLE 3

| | | | | | | | | |
|---|---|---|---|---|---|---|---|---|
| Glycine Rich | | | | | | | | |
| Loop | | | | | | | | |
| Y35 | | | | | | | | |
| alpha-C Helix | | | | | | | | |
| I62 | I63 | K66 | R67 | R70 | E71 | L74 | L75 | M78 |
| Catalytic Loop | | | | | | | | |
| I146 | I147 | H148 | R149 | D150 | | | | |
| C-Lobe | | | | | | | | |
| W197 | M198 | H199 | Y200 | | | | | |

Gsk-3 Beta Kinase

X-ray crystal structural analysis of gsk-3 beta kinase reveals the switch control pocket based on structures 1GNG (SEQ ID NO. 15), 1H8F (SEQ ID NOS. 16 and 17), 1109 (SEQ ID NO. 18) and 109U (SEQ ID NOS. 28 and 29) (Frame et al., Molecular Cell, Vol. 7, pp. 1321-1327 (2001); Dajani et al, Cell, Vol. 105, pp. 721-732 (2001); Dajani et al., EMBO Journal, Vol. 22, pp. 494-501 (2003); and ter Haar, et al., Nature Structural Biology, Vol. 8, pp. 593-596 (2001). The switch control pocket corresponding to the above identified switch control ligand sequences 1 and 2 has a cluster of 2 basic amino acids taken from a combination of the alpha-C helix (residues 96-104), and the catalytic loop (residues 177-186). Specifically, arginine 96 comes from the alpha-C helix, and arginine 180 comes from the catalytic loop. Other amino acids which contribute to the switch control pocket include residues from the glycine rich loop (residues 66-68), amino acids taken from the alpha-C helix (residues 90-104), and amino acids taken from the catalytic loop (residues 177-186). Additionally amino acids from C-lobe residues 233-235 form the base of this pocket.

Table 4 illustrates amino acids from the protein sequence which form the switch control pocket.

TABLE 4

| | | | | | |
|---|---|---|---|---|---|
| Glycine Rich Loop | | | | | |
| F67 | | | | | |
| alpha-C Helix | | | | | |
| R96 | I100 | M101 | L104 | | |
| Catalytic Loop | | | | | |
| I177 | C178 | H179 | R180 | D181 | N186 |
| C-Lobe | | | | | |
| D233 | Y234 | T235 | | | |

Insulin Receptor Kinase-1

X-ray crystal structural analysis of the insulin receptor kinase-1 reveals the switch control pocket based on structures 1 GAG (SEQ ID NOS. 19 and 20) and 1IRK (SEQ ID NO. 21) (Parang et al., Nat. Structural Biology, 8, p. 37 (2001); Hubbard et al., Nature, 372, p. 476 (1994). The switch control pocket for the switch control ligand sequence has a cluster of 2 basic amino acids taken from a combination of the alpha-C helix (residues 1037-1054), and the catalytic loop (residues 1127-1137). Specifically, arginine 1039 is contributed from the alpha-C helix, and arginine 1131 is contributed from the catalytic loop. Other amino acids which contribute to the switch control pocket include residues from the glycine rich loop (residues 1005-1007), amino acids taken from the alpha-C helix (residues 1037-1054), and amino acids taken from the catalytic loop (residues 1127-1137). Additionally amino acids taken from C-lobe residues 1185-1187 form the base of this pocket.

Table 5 illustrates amino acids from the protein sequence which form the switch control pocket.

TABLE 5

| | | | | | | | |
|---|---|---|---|---|---|---|---|
| Glycine Rich Loop | | | | | | | |
| F1007 | | | | | | | |
| alpha-C | | | | | | | |
| Helix | | | | | | | |
| R1039 | E1043 | F1044 | N1046 | E1047 | V1050 | M1051 | F1054 |
| Catalytic | | | | | | | |
| Loop | | | | | | | |
| F1128 | V1129 | H1130 | R1131 | D1132 | | | |
| C-Lobe | | | | | | | |
| V1185 | F1186 | T1187 | | | | | |

Protein Kinase B/Akt

X-ray crystal structural analysis of protein kinase B/Akt reveals the switch control pocket based on structures 1GZK (SEQ ID NO. 22), 1GZO (SEQ ID NO. 23), and 1GZN (SEQ ID NO. 24) (Yang et al, Molecular Cell (2002) 9:1227. The switch control pocket for the corresponding switch control ligand sequence is constituted of amino acid residues taken from the B-helix (residues 185-190), the C helix (residues 194-204) and the beta-5 strand (residues 225-231). In particular, arginine 202 comes from the C-helix.

Table 6 illustrates amino acids from the protein sequence which form the switch control pocket of protein kinase B/Akt.

TABLE 6

| | | | | | | | |
|---|---|---|---|---|---|---|---|
| alpha B-Helix | | | | | | | |
| K185 | E186 | Y187 | I188 | I189 | A190 | | |
| alpha C-Helix | | | | | | | |
| V194 | A195 | H196 | T197 | V198 | T199 | E200 | S201 |
| R202 | V203 | L204 | | | | | |
| B5 strand | | | | | | | |
| L225 | C226 | F227 | V228 | M229 | E230 | Y231 | |

Transforming Growth Factor B-I Receptor Kinase

X-ray crystal structural analysis of the transforming growth factor B-I receptor kinase reveals the switch control pocket, based on structure 1B6C (SEQ ID NO. 25) (Huse et al., Cell (1999) 96:425). The switch control pocket is made up of amino acid residues taken from the GS-1 helix, the GS-2 helix, N-lobe residues 253-266, and alpha-C helix residues 242-252.

Table 7 illustrates amino acids from the protein sequence which form the switch control pocket of TGF B-I receptor kinase.

TABLE 7

| | | | | | | | |
|---|---|---|---|---|---|---|---|
| GS-1 Helix | | | | | | | |
| Y182 | I181 | | | | | | |
| GS-2 Helix | | | | | | | |
| Q198 | | | | | | | |
| N-LOBE | | | | | | | |
| M253 | L254 | R255 | F262 | I263 | A264 | A265 | D266 |
| alpha-C | | | | | | | |
| Helix | | | | | | | |
| | | W242 | F243 | A246 | Y249 | Q250 | V252 |

A second switch control pocket exists in the TGF B-1 receptor kinase. This switch control pocket is similar to the pockets described above for (bcr)abl (Table 1), p38-alpha kinase (Table 3), and gsk-3 beta kinase (Table 4). Although TGF B-1 does not have an obvious complementary switch control ligand to match this pocket, nevertheless this pocket has been evolutionarily conserved and may be used for binding small molecule switch control modulators. This pocket is made up of residues from the Glycine Rich Loop, the alpha-C helix, the catalytic loop, the switch control ligand sequence and the C-lobe.

Table 8 illustrates amino acids from the protein sequence which form this switch control pocket.

TABLE 8

| | | | | | | | |
|---|---|---|---|---|---|---|---|
| Glycine rich Loop | | | | | | | |
| R215 | F216 | | | | | | |
| Lobe | | | | | | | |
| F234 | R237 | | | | | | |
| alpha-C Helix | | | | | | | |
| R244 | S241 | I248 | V252 | | | | |
| Catalytic Loop | | | | | | | |
| I329 | A330 | H331 | R332 | D333 | L334 | | |
| Switch Control | | | | | | | |
| Ligand Sequence | | | | | | | |
| D351 | L352 | G | L | A | V | R | H |
| D351 | S | A | T | D | T | I | D |
| I | A | P | N | H | R | V | |
| C-Lobe | | | | | | | |
| H392 | F393 | E394 | | | | | |

A third switch control pocket is spatially located between the ATP binding pocket and the alpha-C helix and is constituted by residues taken from those identified in Table 9. This pocket is provided as a result of the distortion of the alpha C helix in the “closed form” that binds the inhibitory protein FKBP12 (SEQ ID NO. 26) (see Huse et al, Molecular Cell (2001) 8:671).

Table 9 illustrates the sequence of the third switch control pocket.

TABLE 9

| | | | | | | | |
|---|---|---|---|---|---|---|---|
| Glycine rich Loop | | | | | | | |
| F216 | G217 | V219 | | | | | |
| N-lobe | | | | | | | |
| K232 | F234 | S235 | S236 | L254 | I259 | L260 | G261 |
| F262 | L276 | L278 | S280 | | | | |
| alpha-C Helix | | | | | | | |
| E245 | A246 | I248 | Y249 | V252 | | | |

Step 3. Ascertain the Nature of the Switch Control Ligand-Switch Control Pocket Interaction, and Identify Appropriate Loci for Small Molecule Design.

1. General computational methods. Computer-assisted delineation of switch-control pockets and switch control pocket/ligand interactions utilized modified forms of SurfNet (Laskowsi, R. A, J. Mol. Graph., 1995, 13, 323; PASS; G. Patrick Brady, G. P. Jr.; Stouten, P. F. W., J. Computer-Aided Mol. Des. 2000, 14, 383, Voidoo, G. J. Kleywegt & T. A. Jones (1994) Acta Cryst D50, 178-185; http://www.iucr.ac.uk/journals/acta/tocs/actad/1994/actad5002.html; and Squares; Jiang, F.; Kim, S. -H.; “‘Soft-docking’”: Matching of Molecular Surface Cubes”, J. Mol. Biol. 1991, 219, 79) in tandem with GRASP for pocket visualization (http://trantor.bioc.columbia.edu/grasp/). Panning and docking of small molecule chemotypes into these putative sites employs SoftDock (http://www.scripps.edu/pub/olson-web/doc/autodock/; Morris, G. M.; Goodsell, D. S.; Halliday, R. S.; Huey, R.; Hart, W. E.; Belew, R. K.; Olson, A. J, J. Computational Chemistry, 1998, 19, 1639] and Dock [http://www.cmpharm.ucsf.edu/kuntz/dock.html; Ewing, T. D. A.; Kuntz, I. D., J. Comp. Chem. 1997, 18, 1175] with AMBER-based [http://www.amber.ucsf.edu/amber/amber.html] constrained molecular dynamics as appropriate.

The general approach used by pocket analysis programs is to define gap regions and use these to determine what solvent accessible holes are available on the surface of the protein. Gap regions are either based on spheres or squares and are defined by first filling the region between two or more atoms with spheres or squares (whole and truncated) and then using these to compute a 3D density map which, when contoured, defines the surface of the gap region. The general approach, as taken from the Surfnet users manual is defined for spheres as follows:

a. Two atoms, A and B, have a trial gap sphere placed midway between their van der Waals surfaces and just touching each one.

b. Neighboring atoms are then considered in turn. If any penetrate the gap sphere, the trial gap sphere radius is reduced until it just touches the intruding atom. The process is repeated until all the neighboring atoms have been considered. If the radius of the sphere falls below some predetermined minimum limit (usually 1.0A) it is rejected. Otherwise, the final gap sphere is saved.

c. The procedure is continued until all pairs of atoms have been considered and the gap region is filled with spheres.

d. The spheres are then used to update points on a 3D array of grid-points using a Gaussian function.

e. The update is such that, when the grid is contoured at a contour level of 100.0, the resultant 3D surface corresponds to each gap sphere.

f. When all the spheres have updated the grid, the final 3D contour represents the surface of the interpenetrating gap spheres, and hence defines the extent of the pocket group of atoms comprising the surface pocket.

Those factors that affect the pocket analysis include the spacing of the grid points, the contour level employed, and the minimum and maximum limits of the sphere radii used to pack the gap. In general, the size and shape of a switch control pocket is described as the consensus pocket found by overlaying the computed switch control pockets determined from each individual program.

As noted above, it has been found that the interaction of a switch control ligand and one or more switch control pockets forms what is termed a “composite switch pocket.” This composite switch pocket has a sequence including amino acid residues taken from both the switch control ligand and the switch control pocket(s).

In other cases, the switch control pocket or the composite switch control pocket may overlap with an active site pocket (e.g., the ATP pocket of a kinase) creating a “combined switch control pocket.” These combined switch control pockets can also be useful as loci for binding with small molecules serving as switch control inhibitors.

Of course, the analysis of composite switch pockets and combined switch pockets is carried out using the same techniques as described above in connection with the switch control pockets.

Abl Kinase

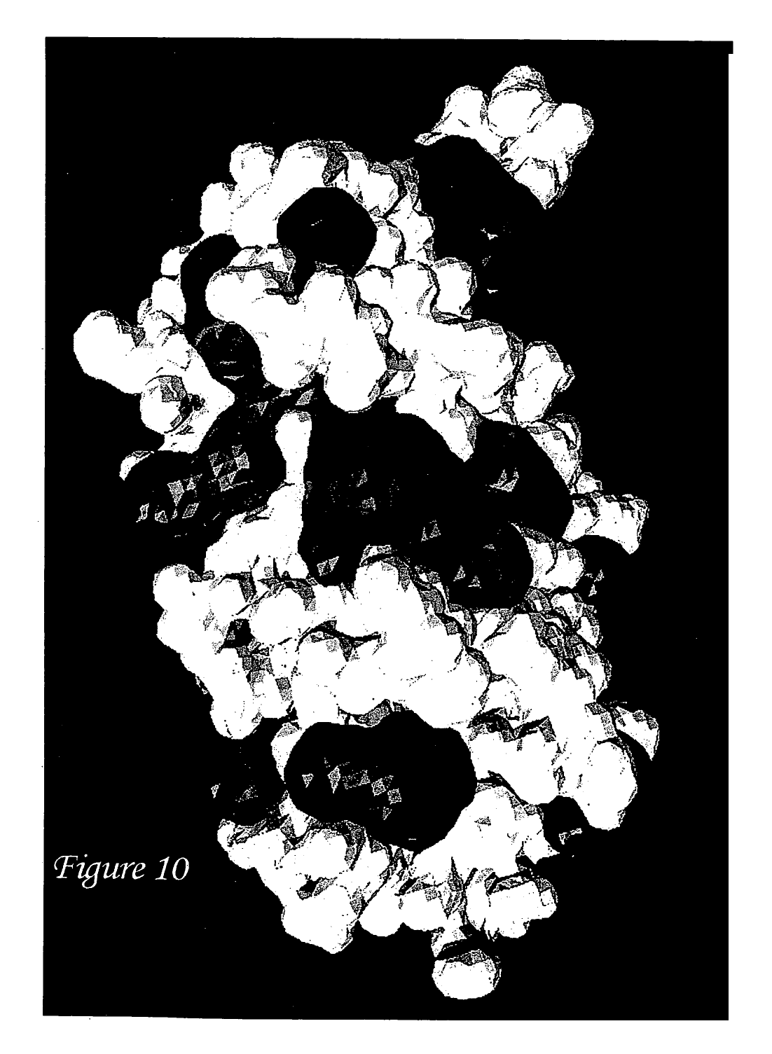

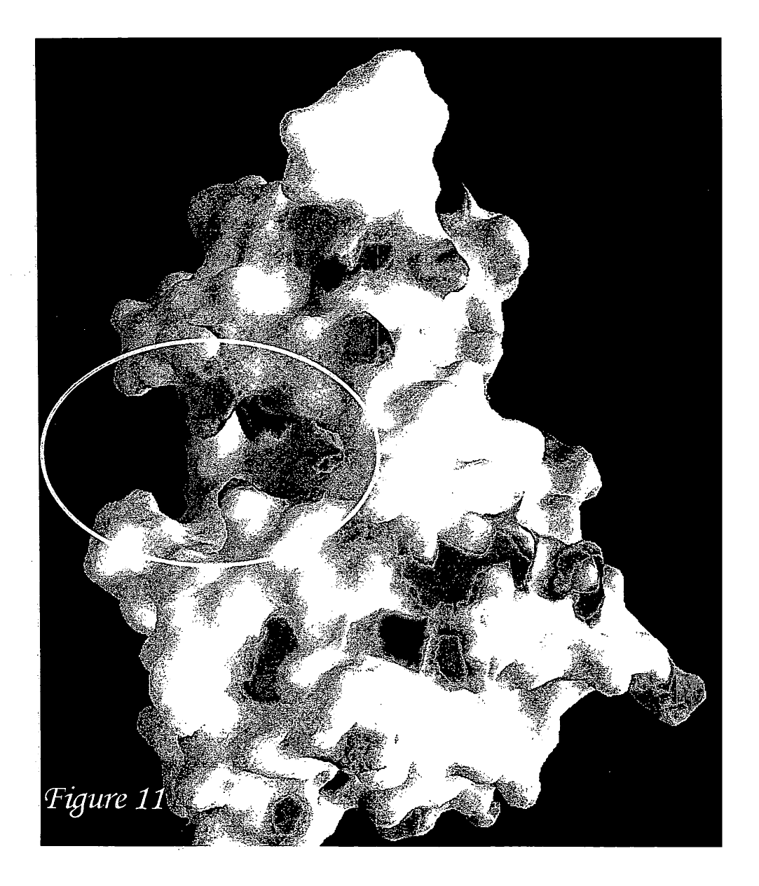

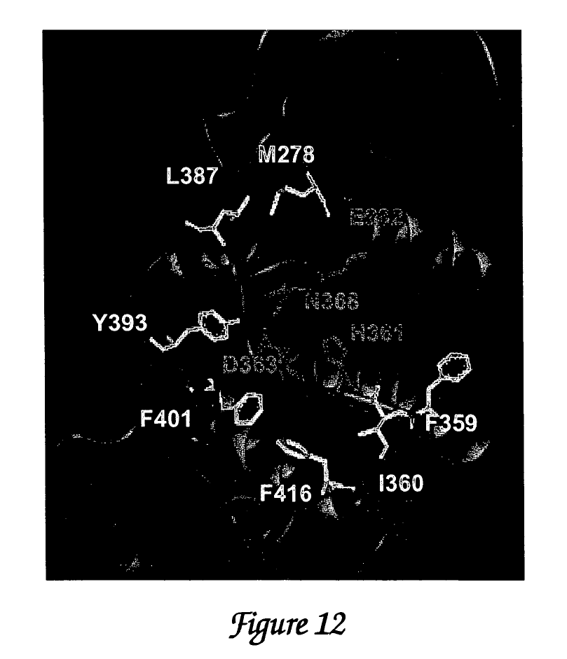

A SURFNET view of the pocket analysis is illustrated in FIG. 10. The switch control pocket is highlighted in light blue. A GRASP view of this switch control pocket is illustrated in FIG. 11, and wherein the composite pocket region of the protein is encircled. FIG. 12 illustrates key amino acid residues which make up the composite switch control pocket of (bcr)abl kinase. The amino acid residues making up the composite pocket are contributed by the switch control ligand and the switch control pocket previously identified. A schematic representation of a composite switch control pocket is depicted in FIG. 6.

The specific amino acid residues making up the composite pocket are set forth in Table 10.

TABLE 10

| | | | | | | B-5 beta | | |
|---|---|---|---|---|---|---|---|---|
| | | | | N-Lobe | | strand | | |
| Glycine Rich Loop | | | | | | | | |
| Y253 | D276 | E279 | | K271 | | I313 | T315 | E316 |
| | | | | M278 | | F317 | M318 | |
| alpha-C Helix | | | | | | | | |
| V280 | E281 | E282 | F283 | L284 | K285 | E286 | A287 | A288 |
| V289 | M290 | | | | | | | |
| alpha-E Helix | F359 | | | | | | | |
| Catalytic Loop | | | | | | | | |
| F359 | I360 | H361 | R362 | D363 | N368 | | | |
| Switch Control Ligand | | | | | | | | |
| Sequence | | | | | | | | |
| D381 | F382 | G383 | L384 | S385 | R386 | L387 | M388 | T389 |
| G390 | D391 | T392 | Y393 | T394 | A395 | H396 | | |
| | | alpha- | | | | | | |
| C-Lobe | | F Helix | | | | | | |
| F401 | | F416 | | | | | | |

The initial small molecule design for this composite switch control pocket focused on chemical probes which would bind to amino acids taken from the N-Lobe beta strand residue (M278), alpha-C helix (E282, K285), the alpha-E helix (F359), the Catalytic Loop (I360, H361, R362, D363, N368), the switch control ligand sequence (R386, L387, Y393), a C-Loop residue (F401) and the alpha-F Helix (F416). Utilization of this composite switch control pocket allowed the design of inhibitors that anchor into this composite switch control pocket of (bcr)abl kinase.

A representative compound selected for screening is N-(4-methyl-3-(4-phenylpyrimidin-2-ylamino)phenyl)-L-4-(2-oxo-4-phenyl-oxazolidinyl-3-carbonyl)benzamide.

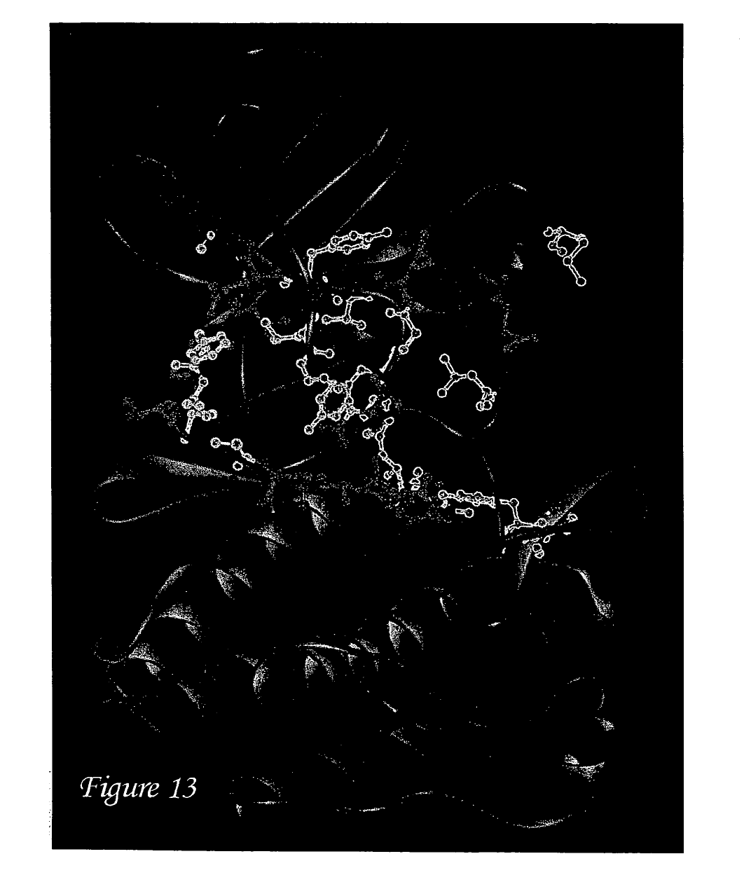

FIG. 13 illustrates key amino acid residues which make up the combined switch control pocket of (bcr)abl kinase. The amino acid residues making up the combined pocket are contributed by the switch control ligand, the switch control pocket, and the ATP active site previously identified. A schematic representation of a combined switch control pocket is depicted in FIG. 7.

The specific amino acid residues making up the combined pocket are set forth in Table 11.

TABLE 11

| | | | | | | B-5 beta | | |
|---|---|---|---|---|---|---|---|---|
| | | | | N-Lobe | | strand | | |
| Glycine Rich Loop | | | | | | | | |
| Y253 | D276 | E279 | | K271 | | I313 | T315 | E316 |
| | | | | | | F317 | M318 | |
| alpha-C Helix | | | | | | | | |
| V280 | E281 | E282 | F283 | L284 | E286 | A287 | A288 | V289 |
| M290 | | | | | | | | |
| Catalytic Loop | | | | | | | | |
| F359 | I360 | H361 | R362 | D363 | | | | |
| Switch Control | | | | | | | | |
| Ligand | | | | | | | | |
| Sequence | | | | | | | | |
| D381 | F382 | G383 | L384 | S385 | R386 | L387 | M388 | T389 |
| G390 | D391 | T392 | Y393 | T394 | A395 | H396 | | |
| | | alpha | | | | | | |
| C-Lobe | | F-Helix | | | | | | |
| F401 | | F416 | | | | | | |
| ATP Pocket | | | | | | | | |
| K247 | L248 | G249 | Q252 | Y253 | G254 | E255 | V256 | Y257 |
| E258 | G259 | V299 | Q300 | L301 | G303 | T315 | E316 | F317 |
| M318 | T319 | G321 | N322 | | | | | |

Utilization of this combined switch control pocket allowed the design of inhibitors that anchor into this combined switch control pocket of (bcr)abl kinase.

Representative compounds selected for screening include: N-[4-methyl-3-(4-pyridin-3-yl-pyrimidin-2-ylamino)-phenyl]-4-(1,1,3-trioxo-[1,2,5]thiadiazolidin-2-ylmethyl)-benzamide; -[4-methyl-3-(4-pyridin-3-yl-pyrimidin-2-ylamino)-phenyl]-D-4-(2-oxo-4-phenyl-oxazolidinyl-3-carbonyl)benzamide; -[4-methyl-3-(4-pyridin-3-yl-pyrimidin-2-ylamino)-phenyl]-L-4-(2-oxo-4-phenyl-oxazolidinyl-3-carbonyl)benzamide; -[4-methyl-3-(4-pyridin-3-yl-pyrimidin-ylamino)-phenyl]-4-(4,4-dioxo-4-thiomorpholinomethyl)benzamide; and N-(3-(4-(pyridin-3-yl)pyrimidin-2-ylamino)-4-methylphenyl)-4-((1-methyl-3,5-dioxo-1,2,4-triazolidin-4-yl)methyl)benzamide.

p38-alpha Kinase

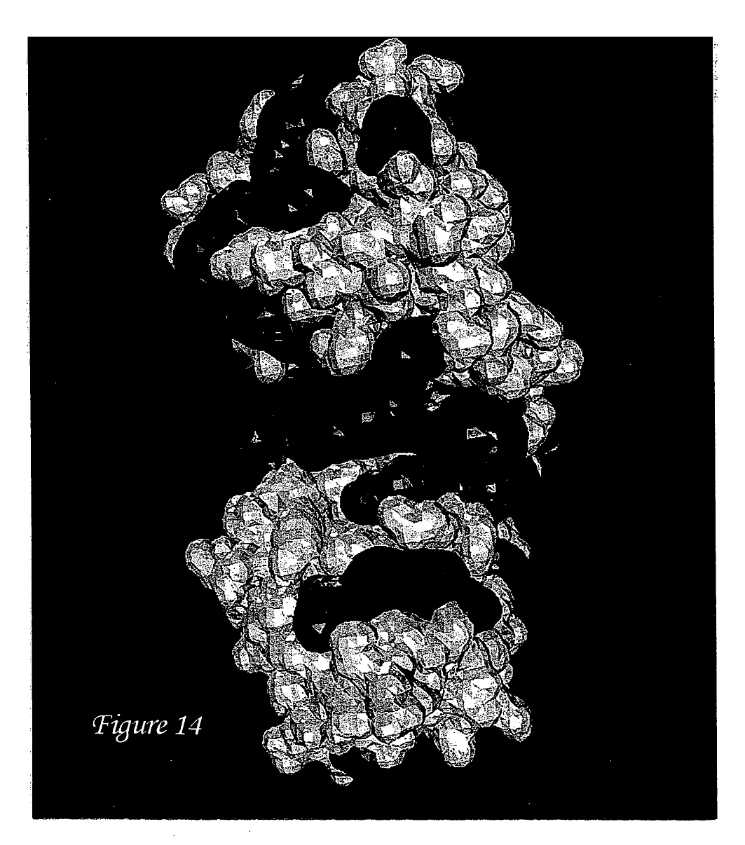

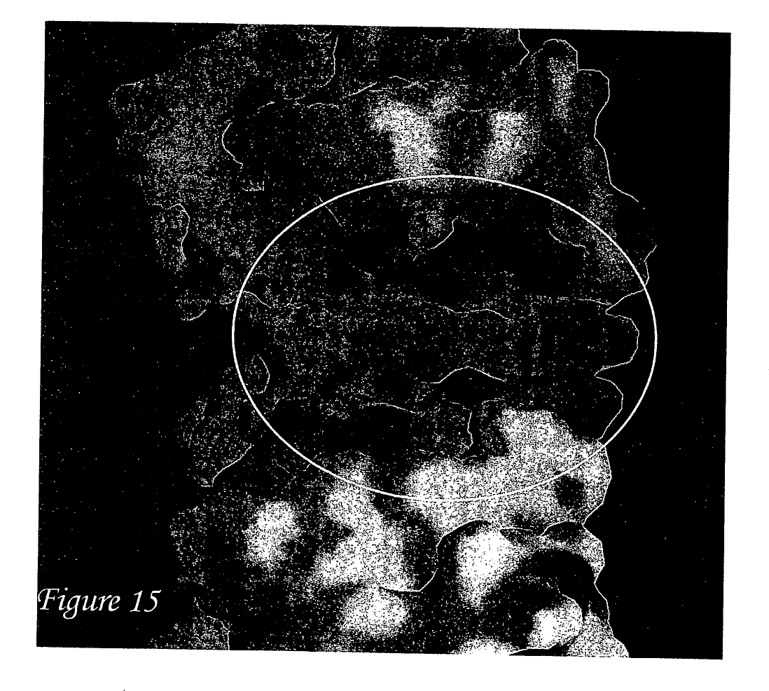

A SURFNET view of the pocket analysis is illustrated in FIG. 14. The composite switch control pocket is highlighted in light blue. A GRASP view of this composite switch control pocket is illustrated in FIG. 15.

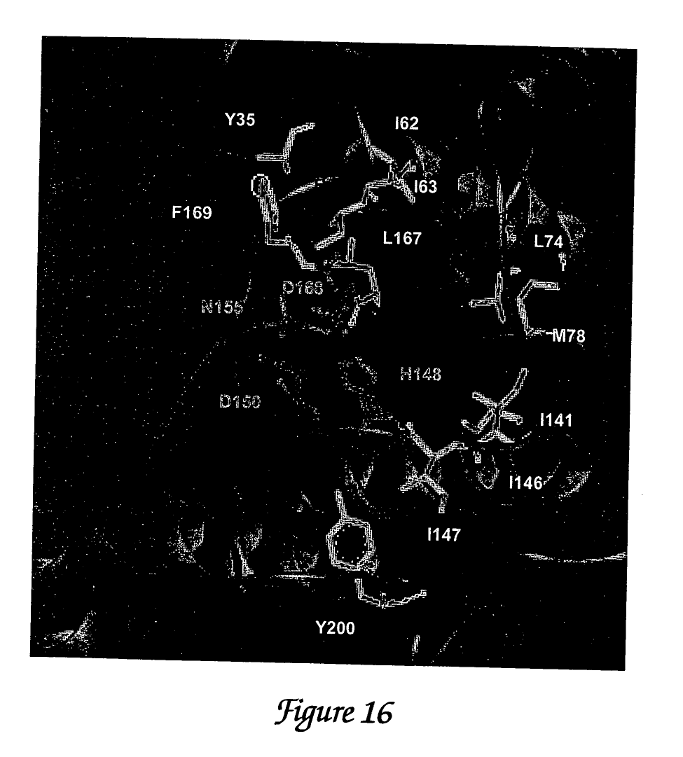

FIG. 16 illustrates key amino acid residues which make up the composite switch control pocket of p38-alpha kinase. These amino acids are taken from the glycine rich loop (Y35), the alpha-C Helix (I62, I63, R67, R70, L74, L75, M78), the alpha-D Helix (I141, I146), the catalytic loop (I147, H148, R149, D150, N155), an N-Lobe strand (L167), the switch control ligand sequence (D168, F169), and the alpha-F Helix (Y200). The specific amino acid residues making up the composite pocket are set forth in the following table:

Table 12 illustrates amino acids from the protein sequence which form the composite switch control pocket.

TABLE 12

| | | | | | | | | |
|---|---|---|---|---|---|---|---|---|
| Glycine | | | | | | | | |
| Rich Loop | | | | | | | | |
| Y35 | | | | | | | | |
| alpha-C | | | | | | | | |
| Helix | | | | | | | | |
| I62 | I63 | K66 | R67 | R70 | E71 | L74 | L75 | M78 |
| Catalytic | | | | | | | | |
| Loop | | | | | | | | |
| I46 | I147 | H148 | R149 | D150 | | | | |
| Switch | | | | | | | | |
| Control | | | | | | | | |
| Ligand | | | | | | | | |
| Sequence | | | | | | | | |
| D168 | F169 | G170 | L171 | A172 | R173 | H174 | T175 | D176 |
| D177 | E178 | M179 | T180 | G181 | Y182 | V183 | A184 | T185 |
| R186 | W187 | Y188 | R189 | | | | | |
| C-Lobe | | | | | | | | |
| W197 | M198 | H199 | Y200 | | | | | |

Utilization of this composite switch control pocket allows the design of inhibitors that anchor into this switch control pocket of p38-alpha kinase.

Representative compounds include: 3-{4-[3-tert-butyl-5-(3-(4-chlorphenyl)ureido-1H-pyrazol-1-yl}phenyl)propanonic acid acid; 3-{4-[3-tert-butyl-5-(3-(naphthalene-1-yl)ureido]-1H-pyrazol-1-yl}phenyl)propanonic acid; 3-(3-{3-tert-butyl-5-[3-(4-chlorophenyl)ureido]-1H-pyrazol-1-yl)phenyl)propionic acid; 3-(3-{3-tert-butyl-5-[3-(naphthalen-1-yl)ureido]-1H-pyrazol-1-yl)phenylpropionic acid; 1-{3-tert-butyl-1-[3-(carbamoylmethyl)phenyl)-1H-pyrazol-5-yl}-3-(4-chlorophenyl)urea; and 1-{3-tert-butyl-1-[3-(2-morpholino-2-oxoethyl)phenyl]-1H-pyrazol-5-yl}-3-(naphthalene-1-yl)urea.

Gsk-3 Beta Kinase

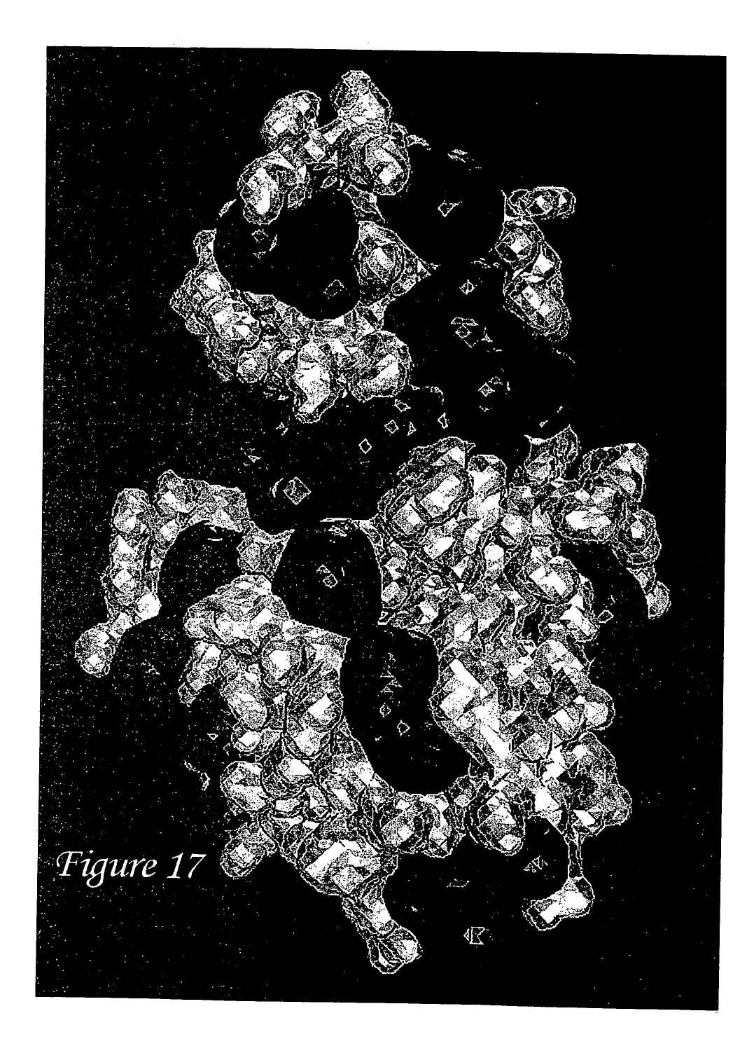

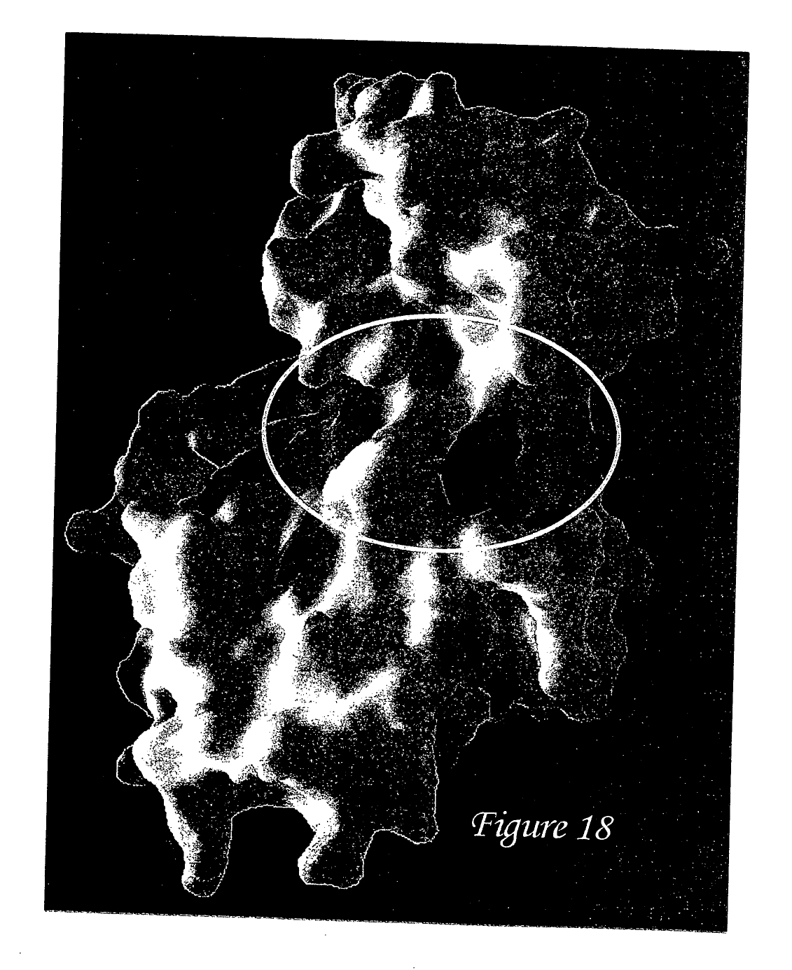

A SURFNET view of the pocket analysis is illustrated in FIG. 17. The composite switch control pocket is highlighted in light blue. A GRASP view of this composite switch control pocket is illustrated in FIG. 18.

FIG. 19 illustrates key amino acid residues which make up the composite switch control pocket of gsk-3 beta kinase. The residues are from the glycine rich loop (F67), the alpha-C Helix (R96, I100, M101, L104), the alpha-D Helix (I141, I146), the catalytic loop (I177, C178, H179, R180, D181, N186), the switch control ligand sequence (D200, F201, S203, K205, L207, V208, P212, N213, V214, Y216), and the alpha-F Helix (Y200). Utilization of this pocket allows the design of small molecule modulator compounds that anchor into this composite switch control pocket of gsk-3 beta kinase.

The composite pocket illustrated in Table 13 is a dual-functionality switch control pocket. When it binds with complemental ligand sequence 1 (Gsk ligand 1) the pocket functions as an on-pocket upregulating protein activity. Alternately, when it binds with complemental ligand sequence 2 (Gsk ligand 2) the pocket functions as an off-pocket downregulating protein activity.

Table 13 illustrates amino acids from the protein sequence which form the composite switch control pocket.

TABLE 13

| | | | | | | | | |
|---|---|---|---|---|---|---|---|---|
| Glycine Rich | | | | | | | | |
| Loop | | | | | | | | |
| F67 | | | | | | | | |
| alpha-C Helix | | | | | | | | |
| R96 | I100 | M101 | L104 | | | | | |
| Catalytic Loop | | | | | | | | |
| I177 | C178 | H179 | R180 | D181 | N186 | | | |
| Switch Control | | | | | | | | |
| Ligand | | | | | | | | |
| Sequence | | | | | | | | |
| D200 | F201 | G202 | S203 | A204 | K205 | Q206 | L207 | V208 |
| R209 | G210 | E211 | P212 | N213 | V214 | S215 | Y216 | I217 |
| C218 | S219 | R220 | | | | | | |
| C-Lobe | | | | | | | | |
| D233 | Y234 | T235 | | | | | | |

Step 4: Express and Purify the Proteins Statically Confined to their Different Switch Controlled States

Gene Synthesis. Genes were completely prepared from synthetic oligonucleotides with codon usage optimized using software (Gene Builder™) provided by Emerald/deCODE genetics, Inc. Whole gene synthesis allowed the codon-optimized version of the gene to be rapidly synthesized. Strategic placement of restriction sites facilitated the rapid inclusion of additional mutations as needed.

The proteins were expressed in baculovirus-infected insect cells or in E. coli expression systems. The genes were optionally modified by incorporating affinity tags that can often allow one-step antibody-affinity purification of the tagged protein. The constructs were optimized for crystallizability, ligand interaction, purification and codon usage. Two 11 Liter Wave Bioreactors for insect cell culture capacity of over 100 L per month were utilized.

Protein purification. For protein purification, an AKTA Purifier, AKTA FPLC, Parr Nitrogen Cavitation Bomb, EmulsiFlex-C5 homogenizer and Protein Maker™ Protein Maker (Emerald's automated parallel purification system) were utilized. Instrumentation for characterizing purified protein included fluorescent spectroscopy, MALDI-ToF mass spectrometry, and dynamic light scattering.

Total cell paste was disrupted by nitrogen cavitation, French press, or microfluidization. The extracts were subjected to parallel protein purification using the Protein Maker™ device. The Protein Maker is a robotic device developed by Emerald that performs simultaneous purification columns in run multiple runs (including Glu-mAb, metal chelate, Q-seph, S-Seph, Phenyl-Seph, and Cibacron Blue) in parallel. The fractions were analyzed by SDS-PAGE. Purified protein was subjected to a number of biophysical assays (Dynamic Light Scattering, UV absorption, MALDI-ToF, analytical gel filtration etc.) to quantitate the level of purity.

Abl Kinase

Whole gene synthesis and subcloning of Abl construct 1 (kinase domain, 6×His-TEV tag, Residues 248-534), Abl construct 2 (kinase domain, Glu-6×His-TEV tag, Residues 248-518), abl construct 3 (kinase domain, Glu-6×His-TEV tag, Residues 248-518, Y412F mutant), abl construct 4 (isoform 1B1-531 with K29R/E30D mutations, TEV-6×His-Glu), and abl construct 5 (isoform 1B1-531 with K29R/E30D/Y412F) was completed and transfections were performed in insect cells. Bcr-abl construct 1 (Glu-6×His-TEV tag, Residues 1-2029) and bcr-abl construct 2 (Glu-6×His-TEV tag, Residues 1-2029; Y412F mutant) were similarly prepared and transfected into insect cells. Fernbach transfection cultures were optionally performed in the presence of the ATP competitive inhibitor PD 180790 or Gleevec to ensure that (bcr) Abl proteins produced were not phosphorylated at Y245 or Y412 (see Tanis et al. Molecular Cell Biology, Vol. 23, p 3884, (2003); Van Etten et al., Journal of Biological Chemistry, Vol. 275, p 35631, (2000)). Protein expression levels was determined by immunoprecipitation and SDS-Page. Protein expression levels for abl Constructs 1 and 2 exceeded 10 mg/L. Py20 (anti-phosphotyrosine antibody) Western blotting was performed on purified protein expressed in the presence of these inhibitors to ensure that Y245 or Y412 were not phosphorylated.

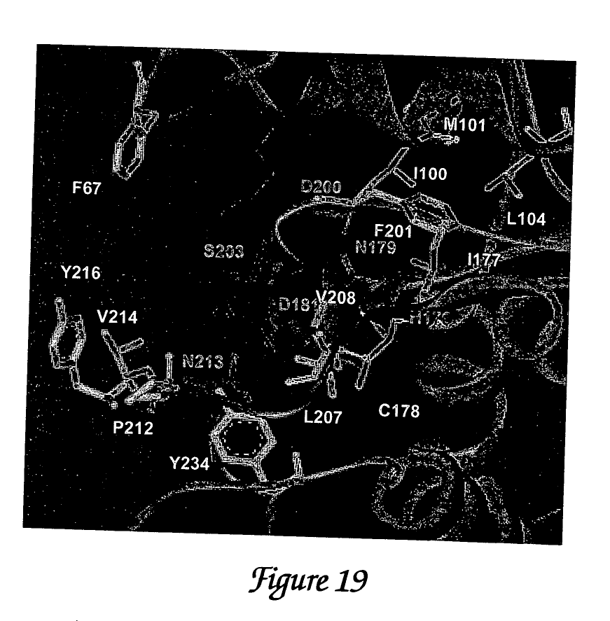

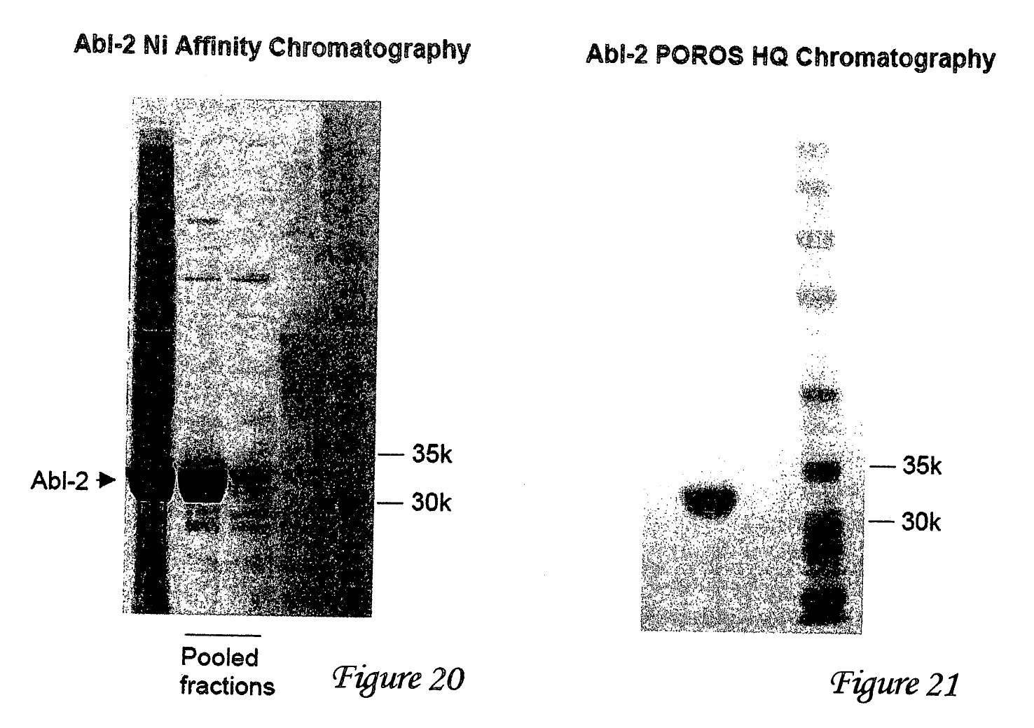

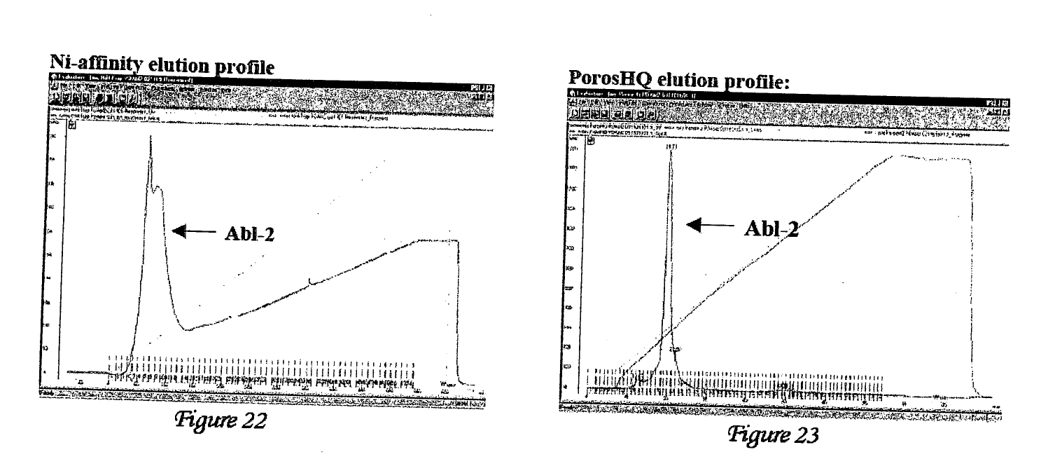

FIGS. 20 and 21 illustrate the purity of abl-construct 2 expressed in the presence of PD180970 after Nickel affinity chromatography (FIG. 20) and subsequent POROS HQ anion exchange chromatography (FIG. 21). FIG. 22 shows the elution profile for abl construct 2 from Nickel affinity chromatography, and FIG. 23 depicts the elution profile for Abl construct 2 from POROS HQ anion exchange chromatography. This form of abl is in its unphosphorylated physical state.

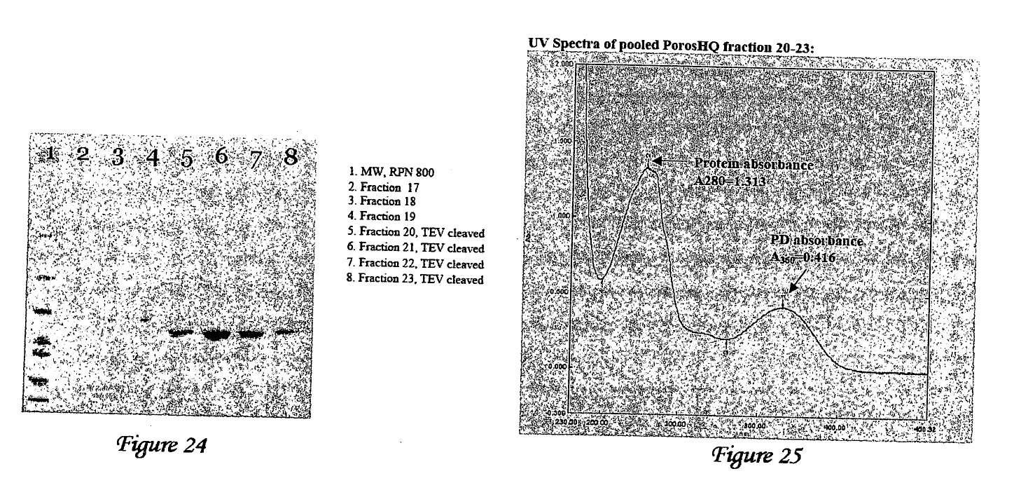

FIG. 24 illustrates the elution profile of Abl construct 2 after treatment with tev protease to remove the Glu-6×His-TEV affinity tag. Fractions 17-19 contain abl protein with the Glu-6×His-TEV tag still intact, while fractions 20-23 contain abl protein wherein the Glu-6×His-TEV tag has been removed. UV analysis (FIG. 25) of the pooled fractions 20-23 revealed an absorbance maximum at 360 nm indicative of the presence of the ATP competitive inhibitor PD 180970 still bound to the abl ATP pocket, thus ensuring the preservation of abl protein in its unphosphorylated state during expression and purification.

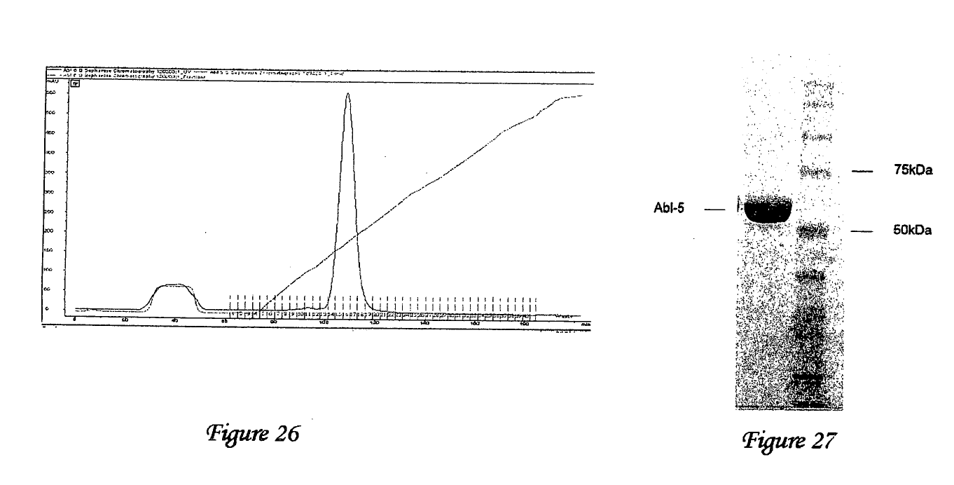

FIG. 26 illustrates the elution profile of abl construct 5 protein abl 1-531, Y412F mutant) upon purification through Nickel affinity chromatography and Q-Sepharose chromatography. FIG. 27 illustrates SDS-Page analysis of purified pooled fractions.

p38-alpha kinase

Whole gene synthesis of p38-alpha kinase construct 1 (6×His-TEV tag, full length) or construct 2 (Glu-6×His-TEV tag, Residues 5-354) was completed and proteins were expressed in E. coli using both arabinose-inducible and T7 promoter vectors. The expression of p38-alpha kinase in two expression vectors (pET15b and pBAD) was examined after induction with 0.5 M IPTG (pET15b) or 0.2% arabinose (pBAD). Protein expression was determined by immunoprecipitation and SDS-Page. Expression of p38-alpha in pBAD constructs after induction was clearly demonstrable in immunoprecipitates with ant-GLU monoclonal antibodies.

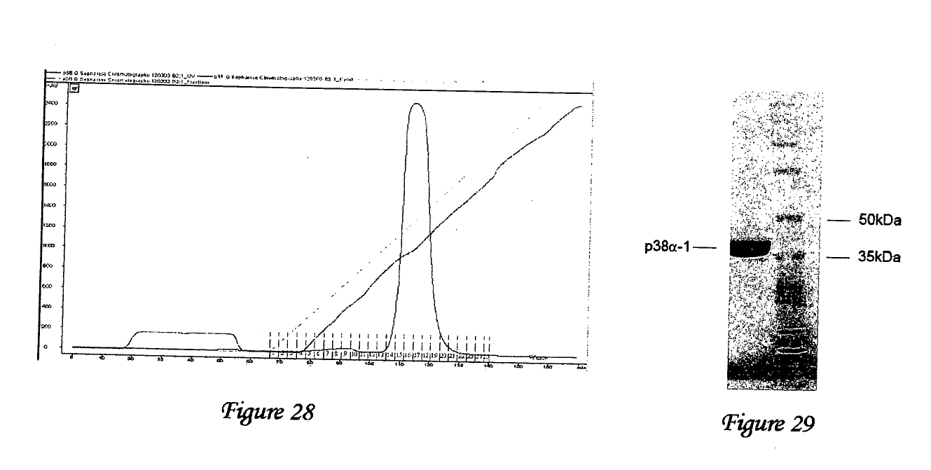

FIG. 28 illustrates the elution profile of p38-alpha protein upon Q-Sepharose chromatography. An SDS-Page of pooled purified fractions is illustrated in FIG. 29.

Gsk-3 Beta Kinase

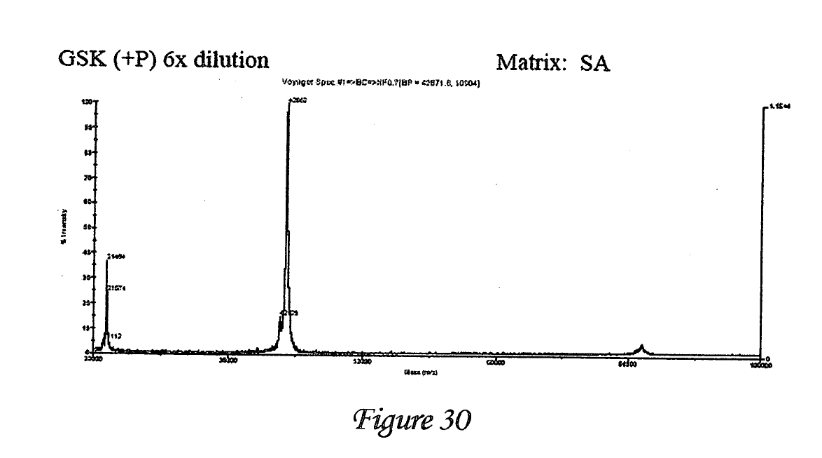

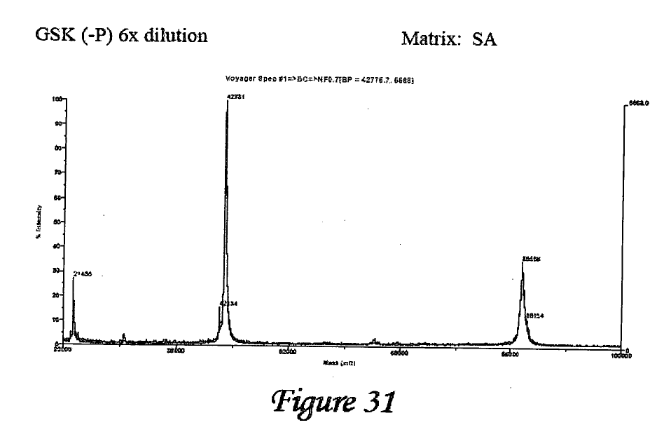

Whole gene synthesis was completed on construct 1 (6×His-TEV tag, full length, same sequence as 1H8F protein), construct 2 (11×His, Residues 27-393, same sequence as 1GNG protein), and construct 3 (Glu-6×His-TEV tag, Residues 35-385). Transfections were performed in insect cells. Protein expression was determined by immunoprecipitation and SDS-Page. The expression level for construct 3 exceeded 5 mg/L. Purification of gsk-3 beta protein involved procedures that allowed isolation of both switch control ligand unphosphorylated kinase (GSK-P) and switch control ligand phosphorylated kinase (GSK+P) forms from the same expression run. Nickel affinity chromatography was performed in 20 mM HEPES buffer at pH7.5. This step was followed by POROS HS (cation-exchange) chromatography. FIG. 30 illustrates the MALDI-TOF spectrum of the GSK+P protein indicating the expected molecular ion of 42862 Da. FIG. 31 illustrates the MADLI-TOF spectrum of the GSK-P protein indicating the expected molecular ion of 42781.

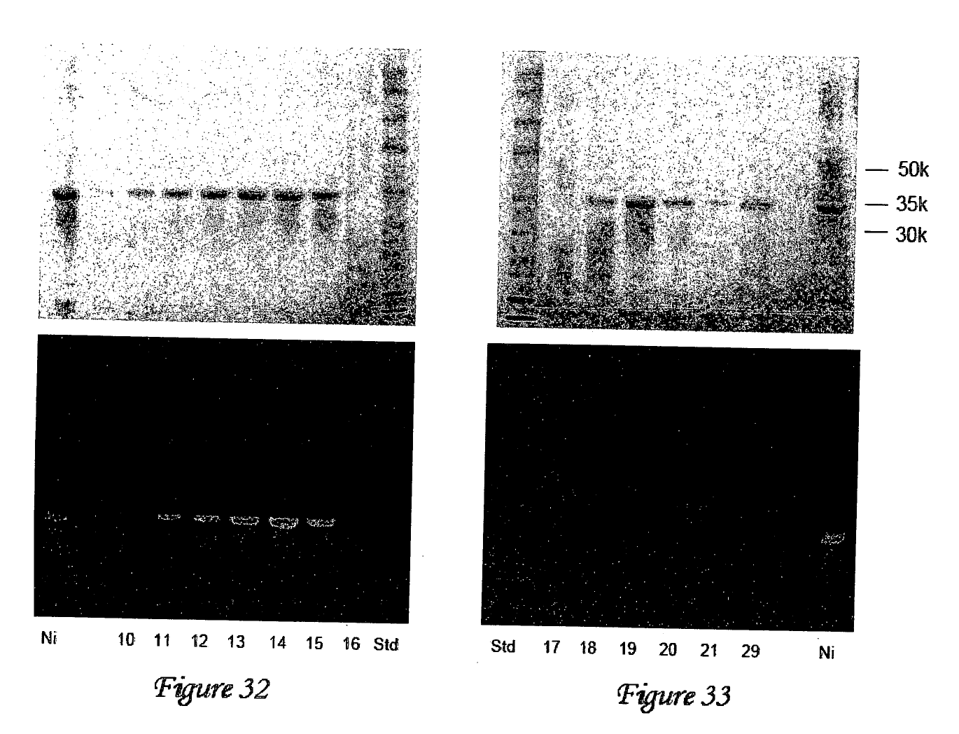

FIGS. 32 and 33 illustrate analysis of POROS HS chromatography fractions by SDS-PAGE analysis in conjunction with staining by the antiphosphotyrosine antibody PY-20. Fractions 10-15 were imaged by the PY-20 antibody, indicating the presence of phosphate on the switch control ligand tyrosine residue. Fractions 17-29 were not imaged by the PY-20 antibody, indicating the absence of switch control ligand phosphorylation of tyrosine.

Step 5. Screening of the Purified Proteins with Candidate Small Molecule Switch Control Modulators

P38-alpha Kinase Screening/P38 MAP Kinase Binding Assay

The binding affinities of small molecule modulators for p38 MAP kinase were determined using a competition assay with SKF 86002 as a fluorescent probe, modified based on published methods (C. Pargellis, et al., Nature Structural Biology (2002) 9, 268-272; J. Regan, et al, J. Med. Chem. (2002)45, 2994-3008). Briefly, SKF 86002, a potent inhibitor of p38 kinase (K_{d}=180 nM), displays an emission fluorescence around 420 nm when excitated at 340 nm upon its binding to the kinase. Thus, the binding affinity of an inhibitor for p38 kinase can be measured by its ability to decrease the fluorescence from SKF 86002. SKF 86002 is a fluoroprobe reagent that serves as a reporter for the integrity of the p38-alpha kinase ATP active site pocket. Small molecule modulators which bind into the switch control pocket of p38-alpha kinase distort the conformation of the protein blocking the ability of the fluorescent probe SKF 86002 to bind. Thus, the ability of a small molecule to block fluoroprobe binding provides an experimental readout of binding to the switch control pocket. Control experiments are performed to determine that the small molecule modulators do not directly compete with fluoroprobe binding by competing at the ATP pocket. The assay was performed in a 384 plate (Greiner nuclear 384 plate) on a Polarstar Optima plate reader (BMG). Typically, the reaction mixture contained 1 μM SKF 86002, 80 nM p38 kinase, and various concentrations of an inhibitor in 20 mM Bis-Tris Propane buffer, pH 7, containing 0.15% (w/v) n-octylglucoside and 2 mM EDTA in a final volume of 65 μl. The reaction was initiated by addition of the enzyme. The plate was incubated at room temperature (˜25° C.) for 2 hours before reading at emission of 420 nm and excitation at 340 nm. By comparison of rfu (relative fluorescence unit) values with that of a control (in the absence of small molecule modulators), the percentage of inhibition at each concentration of the small molecules were calculated. IC₅₀ values for the small molecule modulators were calculated from the % inhibition values obtained at a range of concentrations of the small molecule modulators using Prism. When time-dependent inhibition was assessed, the plate was read at multiple reaction times such as 0.5, 1, 2, 3, 4 and 6 hours. The IC₅₀ values were calculated at each time point. An inhibition was assigned as time-dependent if the IC₅₀ values decrease with the reaction time (more than two-fold in four hours).

TABLE 14

| Example # | IC50, nM | Time-dependent |
|---|---|---|
| 1 | 292 | Yes |
| 2 | 997 | No |
| 2 | 317 | No |
| 3 | 231 | Yes |
| 4 | 57 | Yes |
| 5 | 1107 | No |
| 6 | 238 | Yes |
| 7 | 80 | Yes |
| 8 | 66 | Yes |
| 9 | 859 | No |
| 10 | 2800 | No |
| 11 | 2153 | No |
| 12 | ˜10000 | No |
| 13 | 384 | Yes |
| 15 | 949 | No |
| 19 | ˜10000 | No |
| 21 | 48 | Yes |
| 22 | 666 | No |
| 25 | 151 | Yes |
| 26 | 68 | Yes |
| 29 | 45 | Yes |
| 30 | 87 | Yes |
| 31 | 50 | Yes |
| 32 | 113 | Yes |
| 37 | 497 | No |
| 38 | 508 | No |
| 41 | 75 | Yes |
| 42 | 373 | No |
| 43 | 642 | No |
| 45 | 1855 | No |
| 46 | 1741 | No |
| 47 | 2458 | No |
| 48 | 3300 | No |
| 57 | 239 | Yes |

IC50 values obtained at 2 hours reaction time

Step 6. Confirm Switch Control Mechanism of Protein Modulation

Small molecules that are found to have affinity for the protein or to exhibit functional modulation of protein activity are paced through biochemical studies to determine that binding or functional modulation is non-competitive or un-competitive with natural ligand sites (eg. The ATP site for kinase proteins). This is accomplished using standard Lineweaver-Burk type analyses.

The mode of binding of switch control modulators to the various proteins are determined by Xray crystallography or NMR techniques. The following section outlines the X-ray crystallography techniques used to determine the molecular mode of binding.

Determination of Switch Control Mechanism of Protein Modulation Using X-ray Crystallography Techniques.

1. Crystallization Laboratory: All crystallization trial data is captured using a custom built database software which is used to drive a variety of robotic devices that set up crystallization trials and monitor the results. B. Computer Hardware:Multiple Linux workstations, Windows 2000 servers, and Silicon Graphics O2 workstations.C. X-ray Crystallography Software: HKL2000, includes DENZO and SCALEPACK (X-ray diffraction data processing); MOSFILM; CCP4 suite, includes AMORE, MOLREP and REFMAC (a variety of crystallographic computing operations, including phasing by molecular replacement, MIR, and MAD); SnB for heavy atom location; SHARP (heavy atom phasing program); CNX (a variety of crystallographic computing operations, including model refinement); EPMR (molecular replacement); XtalView (model visualization and building).

2. Crystal Growth and X-ray Diffraction Quality Analysis: Sparse matrix and focused crystallization screens are set up with and without ligands at 2 or more temperatures. Crystals obtained without ligands (apo-crystals) are used for ligand soaking experiments. Once suitable Protein-Crystals have been obtained, a screen is performed to determine the diffraction quality of the Protein-Crystals under various cryo-preservation conditions on an R-AXIS IV imaging plate system and an X-STREAM cryostat. Protein-Crystals of sufficient diffraction quality are used for X-ray diffraction data collection in-house, or stored in liquid nitrogen and saved for subsequent data collection at a synchrotron X-ray radiation source at the COM-CAT beamline at the Advanced Photon Source at Argonne National Laboratory or another synchrotron beam-line. The diffraction limits of Protein-Crystals are determined by taking at least two diffraction images at phi spindle settings 90° apart. The phi spindle are oscillated 1 degree during diffraction image collection. Both images are processed by the HKL-2000 suite of X-ray data analysis and reduction software. The diffraction resolution of the Protein-Crystals are accepted as the higher resolution limit of the resolution shell in which 50% or more of the indexed reflections have an intensity of 1 sigma or greater.

3. X-ray Diffraction Data Collection: A complete data set is defined as having at least 90% of all reflections in the highest resolution shell have been collected. The X-ray diffraction data are processed (reduced to unique reflections and intensities) using the HKL-2000 suite of X-ray diffraction data processing software.

4. Structure Determination: The structures of the Protein-small molecule complexes are determined by molecular replacement (MR) using one or more Protein search models available in the PDB. If necessary, the structure determination is facilitated by multiple isomorphous replacement (MIR) with heavy atoms and/or multi-wavelength anomalous diffraction (MAD) methods. MAD synchrotron data sets are collected for heavy atom soaked crystals if EXAFS scans of the crystals (after having been washed in mother liquor or cryoprotectant without heavy atom) reveal the appropriate heavy atom signal. Analysis of the heavy atom data sets for derivatization are completed using the CCP4 crystallographic suite of computational programs. Heavy atom sites are identified by (|F_{PH}|−|F_{P}|)² difference Patterson and the (|F⁺|−|F⁻|)² anomalous difference Patterson map.

Step 7. Iterate Above Steps to Improve Small Molecule Switch Control Modulators

Individual small molecules found to modulate protein activity are evaluated for affinity and functional modulation of other proteins within the protein superfamily (e.g., other kinases if the candidate protein is a kinase) or between protein families (e.g., other protein classes such as phosphatases and transcription factors if the candidate protein is a kinase). Small molecule screening libraries are also evaluated in this screening paradigm. Structure activity relationships (SARs) are assessed and small molecules are subsequently designed to be more potent for the candidate protein and/or more selective for modulating the candidate protein, thereby minimizing interactions with countertarget proteins.

The analysis of the kinase proteins revealed four types of switch control pockets classified by their mode of binding to complemental switch control ligands, namely: (1) pockets which stabilize and bind to charged ligands, typically formed by phosphorylation of serine, threonine, or tyrosine amino acid residues in the complemental switch control ligands (charged ligand), or by oxidation of the sulfur atoms of methionine or cysteine amino acids; (2) pockets which bind to ligands through the mechanism of hydrogen bonding or hydrophobic interactions (H-bond/hydrophobic ligand); (3) pockets which bind ligands having acylated residues (acylated ligand); and (4) pockets which do not endogenously bind with a ligand, but which can bind with a non-naturally occurring switch control modulator compound (non-identified ligand). Further, these four types of pockets may be of the simple type schematically depicted in FIGS. 1-4, the composite type shown in FIG. 6, or the combined type of FIG. 7. Finally, the pockets may be defined by their switch control functionality, i.e., the pockets may be of the on variety which induces a biologically upregulated protein conformation upon switch control ligand interaction, the off variety which induces a biologically downregulated conformation upon switch control ligand interaction, or what is termed “dual functionality” pockets, meaning that the same pocket serves as both an on-pocket and an off-pocket upon interaction with different complemental switch control ligands. This same spectrum of pockets can be found in all proteins of interest, i.e., those proteins which experience conformational changes via interaction of switch control ligand sequences and complemental switch control pockets.

The following Table 15 further identifies the pockets described in Steps 2 and 3 in terms of pocket classification and type.

TABLE 15

| | Identifying | |
|---|---|---|
| Protein | Table | Switch Control Pocket Type |
| abl kinase | 1 | Charged ligand; Simple; -On |
| abl kinase | 2 | Acylated ligand; Simple; -Off |
| p38-alpha kinase | 3 | Charged ligand; Simple; -On |
| Gsk-3 beta kinase | 4 | Charged ligand; Simple; -Dual |
| Insulin receptor | 5 | Charged ligand; Simple; -On |
| kinase-1 | | |
| Protein kinase B/Akt | 6 | Charged ligand; Simple; -On |
| Transforming Growth | 7 | H-bond/hydrophobic; |
| Factor B-I | | Simple; -Off |
| receptor kinase | | |
| Transforming Growth | 8 | Non-identified ligand |
| Factor B-I | | |
| receptor kinase | | |
| Transforming Growth | 9 | Non-identified ligand |
| Factor B-I | | |
| receptor kinase | | |
| abl kinase | 10 | Charged ligand; Composite; -On |
| abl kinase | 11 | Charged ligand; Combined; -On |
| p38 alpha kinase | 12 | Charged ligand; Composite; -On |
| Gsk-3 beta kinase | 13 | Charged ligand; Composite; -Dual |

A principal aim of the invention is to facilitate the design and development of non-naturally occurring small molecule modulator compounds which will bind with selected proteins at the region of one or more of the switch control pockets thereof in order to modulate the activity of the protein. This functional goal can be achieved in several different ways, depending upon the type of switch control pocket (on, -off, or -dual), the nature of the selected modulator compound, and the type of interactive binding between the modulator compound and the protein.

For example, a selected modulator compound may bind at the region of a selected switch control pocket as a switch control ligand agonist, i.e., the modulator compound effects the same type of conformational change as that induced by the naturally occurring, complemental switch control ligand. Thus, if a switch control ligand agonist binds with an on-pocket, the result will be upregulation of the protein activity, and if it binds with an off-pocket, downregulation occurs.

Conversely, a given modulator may bind as a switch control ligand antagonist, i.e., the modulator compound effects the opposite type of conformational change as that induced by the naturally occurring, complemental switch control ligand. Hence, if a switch control ligand antagonist binds with an on-pocket, the result will be downregulation of the protein activity, and if it binds with an off-pocket, upregulation occurs.

In the case of dual functionality and non-identified liganded pockets, a modulator compound serves as a functional agonist or functional antagonist, depending upon on the type of response obtained.

### EXAMPLE 2

### Synthesis of Potential Switch Control Small Molecules

The following examples set forth the synthesis of compounds particularly useful as candidates for switch control molecules designed to interact with kinase proteins. In these examples, those designated with letters refer to synthesis of intermediates, whereas those designated with numbers refer to synthesis of the final compounds.

[Boc-sulfamide] aminoester (Reagent AA), 1,5,7,-trimethyl-2,4-dioxo-3-aza-bicyclo[3.3.1]nonane-7-carboxylic acid (Reagent BB), and Kemp acid anhydride (Reagent CC) was prepared according to literature procedures. See Askew et. al J. Am. Chem. Soc. 1989, 111, 1082 for further details.

### EXAMPLE A

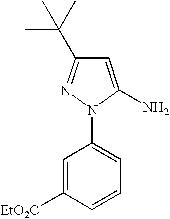

To a solution (200 mL) of m-amino benzoic acid (200 g, 1.46 mol) in concentrated HCl was added an aqueous solution (250 mL) of NaNO₂ (102 g, 1.46 mol) at 0° C. The reaction mixture was stirred for 1 h and a solution of SnCl₂.2H₂O (662 g, 2.92 mol) in concentrated HCl (2 L) was then added at 0° C., and the reaction stirred for an additional 2 h at RT. The precipitate was filtered and washed with ethanol and ether to yield 3-hydrazino-benzoic acid hydrochloride as a white solid.

The crude material from the previous reaction (200 g, 1.06 mol) and 4,4-dimethyl-3-oxo-pentanenitrile (146 g, 1.167 mol) in ethanol (2 L) were heated to reflux overnight. The reaction solution was evaporated in vacuo and the residue purified by column chromatography to yield ethyl 3-(3-tert-butyl-5-amino-1H-pyrazol-1-yl)benzoate (Example A, 116 g, 40%) as a white solid together with 3-(5-amino-0.3-tert-butyl-1H-pyrazol-1-yl)benzoic acid (93 g, 36%). ¹H NMR (DMSO-d₆): 8.09 (s, 1H), 8.05 (brd, J=8.0 Hz, 1H), 7.87 (brd, J=8.0 Hz, 1H), 7.71 (t, J=8.0 Hz, 1H), 5.64 (s, 1H), 4.35 (q, J=7.2 Hz, 2H), 1.34 (t, J=7.2 Hz, 3H), 1.28 (s, 9H).

### EXAMPLE B

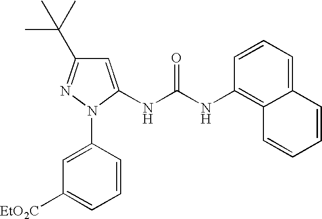

To a solution of 1-naphthyl isocyanate (9.42 g, 55.7 mmol) and pyridine (44 mL) in THF (100 mL) was added a solution of Example A (8.0 g, 27.9 mmol) in THF (200 mL) at 0° C. The mixture was stirred at RT for 1 h, heated until all solids were dissolved, stirred at RT for an additional 3 h and quenched with H₂O (200 mL). The precipitate was filtered, washed with dilute HCl and H₂O, and dried in vacuo to yield ethyl 3-[3-t-butyl-5-(3-naphthalen-1-yl)ureido)-1H-pyrazol-1-yl]benzoate(12.0 g, 95%) as a white power. ¹H NMR (DMSO-d₆): 9.00 (s, 1H), 8.83 (s, 1 H), 8.25 7.42 (m, 11 H), 6.42 (s, 1 H), 4.30 (q, J=7.2 Hz, 2 H), 1.26 (s, 9 H), 1.06 (t, J=7.2 Hz, 3 H); MS (ESI) m/z: 457.10 (M+H⁺).

### EXAMPLE C

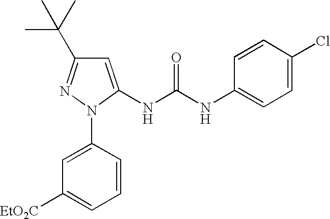

To a solution of Example A (10.7 g, 70.0 mmol) in a mixture of pyridine (56 mL) and THF (30 mL) was added a solution of 4-nitrophenyl 4-chlorophenylcarbamate (10 g, 34.8 mmol) in THF (150 mL) at 0° C. The mixture was stirred at RT for 1 h and heated until all solids were dissolved, and stirred at RT for an additional 3 h. H₂O (200 mL) and CH₂Cl₂ (200 mL) were added, the aqueous phase separated and extracted with CH₂Cl₂ (2×100 mL). The combined organic layers were washed with 1N NaOH, and 0.1N HCl, saturated brine and dried over anhydrous Na₂SO₄. The solvent was removed in vacuo to yield ethyl 3-{3-tert-butyl-5-[3-(4-chlorophenyl)ureido]-1H-pyrazol-1-yl}benzoate (8.0 g, 52%). ¹H NMR (DMSO-d₆): δ 9.11 (s, 1H), 8.47 (s, 1H), 8.06 (m, 1H), 7.93 (d, J=7.6 Hz, 1H), 7.81 (d, J=8.0 Hz, 1H), 7.65 (dd, J=8.0, 7.6 Hz, 1H), 7.43 (d, J=8.8 Hz, 2H), 7.30 (d, J=8.8 Hz, 2H), 6.34 (s, 1H), 4.30 (q, J=6.8 Hz, 2H), 1.27 (s, 9H), 1.25 (t, J=6.8 Hz, 3H); MS (ESI) m/z: 441 (M⁺+H).

### EXAMPLE D

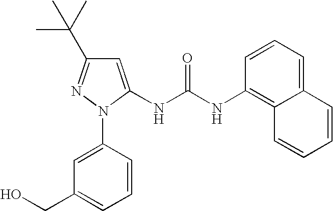

To a stirred solution of Example B (8.20 g, 18.0 mmol) in THF (500 mL) was added LiAlH₄ powder (2.66 g, 70.0 mmol) at −10° C. under N₂. The mixture was stirred for 2 h at RT and excess LiAlH₄ destroyed by slow addition of ice. The reaction mixture was acidified to pH=7 with dilute HCl, concentrated in vacuo and the residue extracted with EtOAc. The combined organic layers were concentrated in vacuo to yield 1-{3-tert-butyl-1-[3-(hydroxymethyl)phenyl]-1H-pyrazol-5-yl}-3-(naphthalen-1-yl)urea (7.40 g, 99%) as a white powder. ¹H NMR (DMSO-d₆): 9.19 (s, 1 H), 9.04 (s, 1 H), 8.80 (s, 1 H), 8.26-7.35 (m, 11 H), 6.41 (s, 1 H), 4.60 (s, 2 H), 1.28 (s, 9 H); MS (ESI) m/z: 415 (M+H⁺).

### EXAMPLE E

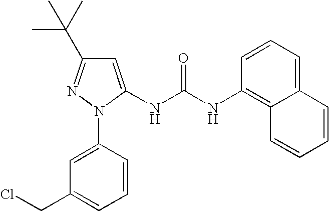

A solution of Example C (1.66 g, 4.0 mmol) and SOCl₂ (0.60 mL, 8.0 mmol) in CH₃Cl (100 mL) was refluxed for 3 h and concentrated in vacuo to yield 1-{3-tert-butyl-1-[3-chloromethyl)phenyl]-1H-pyrazol-5-yl}-3-(naphthalen-1-yl)urea (1.68 g, 97%) was obtained as white powder. ¹H NMR (DMSO-d₆): δ 9.26 (s, 1 H), 9.15 (s, 1 H), 8.42-7.41 (m, 11 H), 6.40 (s, 1 H), 4.85 (s, 2 H), 1.28 (s, 9 H). MS (ESI) m/z: 433 (M+H⁺).

### EXAMPLE F

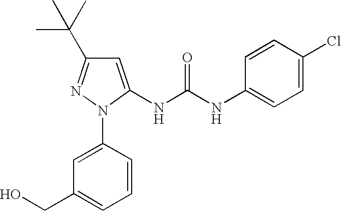

To a stirred solution of Example C (1.60 g, 3.63 mmol) in THF (200 mL) was added LiAlH₄ powder (413 mg, 10.9 mmol) at −10° C. under N₂. The mixture was stirred for 2 h and excess LiAlH₄ was quenched by adding ice. The solution was acidified to pH=7 with dilute HCl. Solvents were slowly removed and the solid was filtered and washed with EtOAc (200+100 mL). The filtrate was concentrated to yield 1-{3-tert-butyl-1-[3-hydroxymethyl)phenyl]-1H-pyrazol-5-yl}-3-(4-chlorophenyl)urea (1.40 g, 97%). ¹H NMR (DMSO-d₆): δ 9.11 (s, 1H), 8.47 (s, 1H), 7.47-7.27 (m, 8H), 6.35 (s, 1H), 5.30 (t, J=5.6 Hz, 1H), 4.55 (d, J=5.6 Hz, 2H), 1.26 (s, 9H); MS (ESI) m/z: 399 (M+H⁺).

### EXAMPLE G

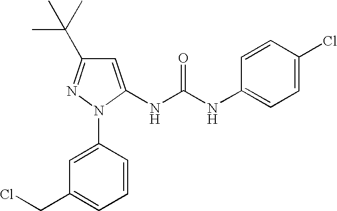

A solution of Example F (800 mg, 2.0 mmol) and SOCl₂ (0.30 mL, 4 mmol) in CHCl₃ (30 mL) was refluxed gently for 3 h. The solvent was evaporated in vacuo and the residue was taken up to in CH₂Cl₂ (2×20 mL). After removal of the solvent, 1-{3-tert-butyl-1-[3-(chloromethyl)phenyl]-1H-pyrazol-5-yl}-3-(4-chlorophenyl)urea (812 mg, 97%) was obtained as white powder. ¹H NMR (DMSO-d₆): δ 9.57 (s, 1H), 8.75 (s, 1H), 7.63 (s, 1H), 7.50-7.26 (m, 7H), 6.35 (s, 1H), 4.83 (s, 2H), 1.27 (s, 9H); MS (ESI) m/z: 417 (M+H⁺).

### EXAMPLE H

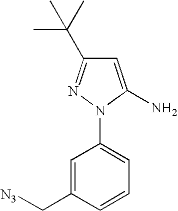

To a suspension of LiAlH₄ (5.28 g, 139.2 mmol) in THF (1000 mL) was added Example A (20.0 g, 69.6 mmol) in portions at 0° C. under N₂. The reaction mixture was stirred for 5 h, quenched with 1 N HCl at 0° C. and the precipitate was filtered, washed by EtOAc and the filtrate evaporated to yield [3-(5-amino-3-tert-butyl-1H-pyrazol-1-yl)phenyl]methanol (15.2 g, 89%). ¹H NMR (DMSO-d₆): 7.49 (s, 1H), 7.37 (m, 2H), 7.19 (d, J=7.2 Hz, 1H), 5.35 (s, 1H), 5.25 (t, J=5.6 Hz, 1H), 5.14 (s, 2H), 4.53 (d, J=5.6 Hz, 2H), 1.19 (s, 9H); MS (ESI) m/z: 246.19 (M+H⁺).

The crude material from the previous reaction (5.0 g, 20.4 mmol) was dissolved in dry THF (50 mL) and SOCl₂ (4.85 g, 40.8 mmol), stirred for 2 h at RT, concentrated in vacuo to yield 3-tert-butyl-1-(3-chloromethylphenyl)-1H-pyrazol-5-amine (5.4 g), which was added to N₃ (3.93 g, 60.5 mmol) in DMF (50 mL). The reaction mixture was heated at 30° C. for 2 h, poured into H₂O (50 mL), and extracted with CH₂Cl₂. The organic layers were combined, dried over MgSO₄, and concentrated in vacuo to yield crude 3-tert-butyl-1-[3-(azidomethyl)phenyl]-1H-pyrazol-5-amine (1.50 g, 5.55 mmol).

### EXAMPLE I

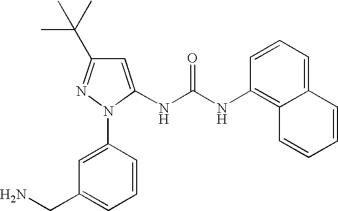

Example H was dissolved in dry THF (10 mL) and added a THF solution (10 mL) of 1-isocyano naphthalene (1.13 g, 6.66 mmol) and pyridine (5.27 g, 66.6 mmol) at RT. The reaction mixture was stirred for 3 h, quenched with H₂O (30 mL), the resulting precipitate filtered and washed with 1N HCl and ether to yield 1-[2-(3-azidomethyl-phenyl)-5-t-butyl-2H-pyrazol-3-yl]-3-naphthalen-1-yl-urea (2.4 g, 98%) as a white solid.

The crude material from the previous reaction and Pd/C (0.4 g) in THF (30 mL) was hydrogenated under 1 atm at RT for 2 h. The catalyst was removed by filtration and the filtrate concentrated in vacuo to yield 1-{3-tert-butyl-1-[3-(amonomethyl)phenyl}-1H-pyrazol-5yl)-3-(naphthalene-1-yl)urea (2.2 g, 96%) as a yellow solid. ¹H NMR (DMSO-d₆): 9.02 (s, 1H), 7.91 (d, J=7.2 Hz, 1H), 7.89 (d, J=7.6 Hz, 2H), 7.67-7.33 (m, 9H), 6.40 (s, 1H), 3.81 (s, 2H), 1.27 (s, 9H); MS (ESI) m/z: 414 (M+H⁺).

### EXAMPLE J

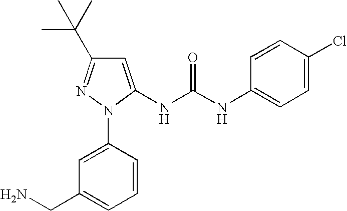

To a solution of Example H (1.50 g, 5.55 mmol) in dry THF (10 mL) was added a THF solution (10 mL) of 4-chlorophenyl isocyanate (1.02 g, 6.66 mmol) and pyridine (5.27 g, 66.6 mmol) at RT. The reaction mixture was stirred for 3 h and then H₂O (30 mL) was added. The precipitate was filtered and washed with 1N HCl and ether to give 1-{3-tert-butyl-1-[3-(amonomethyl)phenyl}-1H-pyrazol-5yl)-3-(4-chlorophenyl)urea (2.28 g, 97%) as a white solid, which was used for next step without further purification. MS (ESI) m/z: 424 (M+H⁺).

### EXAMPLE K

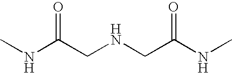

To a solution of benzyl amine (16.5 g, 154 mmol) and ethyl bromoacetate (51.5 g, 308 mmol) in ethanol (500 mL) was added K₂CO₃ (127.5 g, 924 mmol). The mixture was stirred at RT for 3 h, was filtered, washed with EtOH, concentrated in vacuo and chromatographed to yield N-(2-ethoxy-2-oxoethyl)-N-(phenylmethyl)-glycine ethyl ester (29 g, 67%). ¹H NMR (CDCl₃): δ 7.39-7.23 (m, 5H), 4.16 (q, J=7.2 Hz, 4H), 3.91(s, 2H), 3.54 (s, 4H), 1.26 (t, J=7.2 Hz, 6H); MS (ESI): m/e: 280 (M⁺+H).

A solution of N-(2-ethoxy-2-oxoethyl)-N-(phenylmethyl)-glycine ethyl ester (7.70 g, 27.6 mmol) in methylamine alcohol solution (25-30%, 50 mL) was heated to 50° C. in a sealed tube for 3 h, cooled to RT and concentrated in vacuo to yield N-(2-methylamino-2-oxoethyl)-N-(phenylmethyl)-glycine methylamide in quantitative yield (7.63 g). ¹H NMR (CDCl₃): δ 7.35-7.28 (m, 5H), 6.75 (br s, 2H), 3.71(s, 2H), 3.20 (s, 4H), 2.81 (d, J=5.6 Hz, 6H); MS (ESI) m/e 250(M+H⁺).

The mixture of N-(2-methylamino-2-oxoethyl)-N-(phenylmethyl)-glycine methylamide (3.09 g, 11.2 mmol) in MeOH (30 mL) was added 10% Pd/C (0.15 g). The mixture was stirred and heated to 40° C. under 40 psi H₂ for 10 h, filtered and concentrated in vacuo to yield N-(2-methylamino-2-oxoethyl)-glycine methylamide in quantitative yield (1.76 g). ¹H NMR (CDCl₃): δ 6.95(br s, 2H), 3.23 (s, 4H), 2.79 (d, J=6.0, 4.8 Hz), 2.25(br s 1H); MS (ESI) m/e 160(M+H⁺)

### EXAMPLE 1

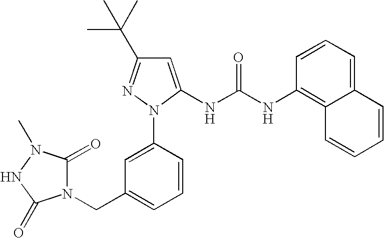

To a solution of 1-methyl-[1,2,4]triazolidine-3,5-dione (188 mg, 16.4 mmol) and sodium hydride (20 mg, 0.52 mmol) in DMSO (1 mL) was added Example E (86 mg, 0.2 mmol). The reaction was stirred at RT overnight, quenched with H₂O (10 mL), extracted with CH₂Cl₂, and the organic layer was separated, washed with brine, dried over Na₂SO₄ and concentrated in vacuo. The residue was purified by preparative HPLC to yield 1-(3-tert-butyl-1-{3-[(1-methyl-3,5-dioxo-1,2,4-triazolidin-4-yl)methyl]phenyl}-1H-pyrazol-5-yl)-3-(naphthalene-1-yl)urea (Example 1, 14 mg). ¹H NMR (CD₃OD): δ7.88-7.86 (m, 2H), 7.71-7.68 (m, 2H), 7.58 (m, 2H), 7.60-7.42 (m, 5H), 6.49 (s, 1H), 4.85 (s, 1H), 1.34 (s, 9H), 1.27 (s, 6H); MS (ESI) m/z: 525 (M+H⁺).

### EXAMPLE 2

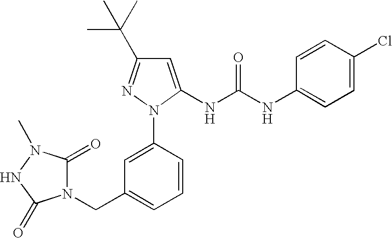

The title compound was synthesized in a manner analogous to Example 1, utilizing Example G to yield 1-(3-tert-butyl-1-{3-[(1-methyl-3,5-dioxo-1,2,4-triazolidin-4-yl)methyl]phenyl}-1H-pyrazol-5-yl)-3-(4-chlorophenyl)urea ¹H NMR (CD₃OD): δ 7.2˜7.5 (m, 7H), 6.40 (s 1H), 4.70 (s, 2H), 2.60 (d, J=14 Hz, 2H), 1.90 (m, 1H), 1.50 (m, 1H), 1.45 (s, 9H), 1.30 (m, 2H), 1.21 (s, 3H), 1.18 (s, 6H); MS (ESI) m/z: 620 (M+H⁺).

### EXAMPLE 3

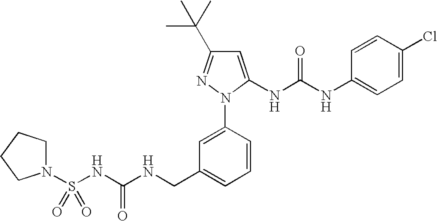

A mixture of compound 1,1-Dioxo-[1,2,5]thiadiazolidin-3-one (94 mg, 0.69 mmol) and NaH (5.5 mg, 0.23 mmol) in THF (2 mL) was stirred at −10° C. under N₂ for 1 h until all NaH was dissolved. Example E (100 mg, 0.23 mmol) was added and the reaction was allowed to stir at RT overnight, quenched with H₂O, and extracted with CH₂Cl₂. The combined organic layers were concentrated in vacuo and the residue was purified by preparative HPLC to yield 1-(3-tert-butyl-1-{[3-(1,1,3-trioxo-[1,2,5]thiadiazolidin-2-yl)methyl]phenyl}-1H-pyrazol-5-yl)-3-(naphthalen-1-yl)urea (18 mg) as a white powder. ¹H NMR (CD₃OD): δ 7.71-7.44 (m, 11 H), 6.45 (s, 1 H), 4.83 (s, 2 H), 4.00 (s, 2 H), 1.30 (s, 9 H). MS (ESI) m/z: 533.40 (M+H⁺).

### EXAMPLE 4

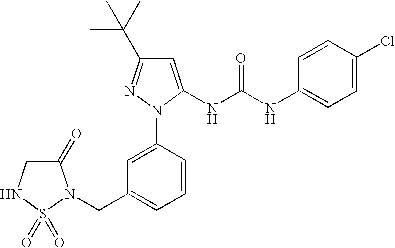

The title compound was obtained in a manner analogous to Example 3 utilizing Example G. to yield 1-(3-tert-butyl-1-{[3-(1,1,3-trioxo-[1,2,5]thiadiazolidin-2-yl)methyl]phenyl}-1H-pyrazol-5-yl)-3-(4-chlorophenyl)urea. ¹H NMR (CD₃OD): δ 7.38-7.24 (m, 8 H), 6.42 (s, 1 H), 4.83 (s, 2 H), 4.02 (s, 2 H), 1.34 (s, 9 H); MS (ESI) m/z: 517 (M+H⁺).

### EXAMPLE 5

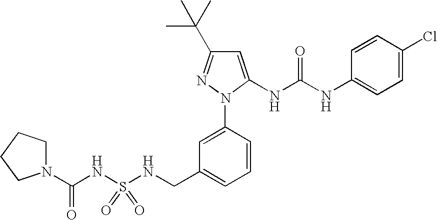

To a stirred solution of chlorosulfonyl isocyanate (19.8 μL, 0.227 mmol) in CH₂Cl₂ (0.5 mL) at 0° C. was added pyrrolidine (18.8 μL, 0.227 mmol) at such a rate that the reaction solution temperature did not rise above 5° C. After stirring for 1.5 h, a solution of Example J (97.3 mg, 0.25 mmol) and Et₃N (95 μL, 0.678 mmol) in CH₂Cl₂ (1.5 mL) was added at such a rate that the reaction temperature didn rise above 5° C. When the addition was completed, the reaction solution was warmed to RT and stirred overnight. The reaction mixture was poured into 10% HCl, extracted with CH₂Cl₂, the organic layer washed with saturated NaCl, dried over MgSO₄, and filtered. After removal of the solvents, the crude product was purified by preparative HPLC to yield 1-(3-tert-butyl-1-[[3-N-[[(1-pyrrolidinylcarbonyl)amino]sulphonyl]aminomethyl]phenyl]-1H-pyrazol-5-yl)-3-(4-chlorophenyl)urea. ¹H NMR(CD₃OD): δ 7.61 (s, 1 H), 7.43-7.47 (m, 3 H), 7.23-7.25 (dd, J=6.8 Hz, 2 H), 7.44 (dd, J=6.8 Hz, 2 H), 6.52 (s, 1 H), 4.05 (s, 2 H), 3.02 (m, 4 H), 1.75 (m, 4 H), 1.34 (s, 9 H); MS (ESI) m/z: 574.00 (M+H⁺).

### EXAMPLE 6

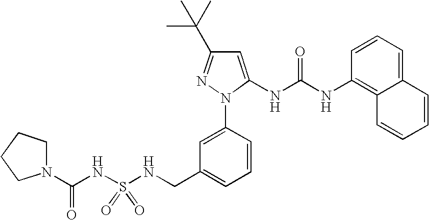

The title compound was made in a manner analogous to Example 5 utilizing Example I to yield 1-(3-tert-butyl-1-[[3-N-[[(1-pyrrolidinylcarbonyl)amino]sulphonyl]aminomethyl]-phenyl]-1H-pyrazol-5-yl)-3-(naphthalen-1-yl)urea. ¹H NMR (CDCl₃): δ 7.88 (m, 2 H), 7.02-7.39 (m, 2 H), 7.43-7.50 (m, 7 H), 6.48 (s, 1 H), 4.45 (s, 1 H), 3.32-3.36 (m, 4 H), 1.77-1.81 (m, 4 H), 1.34 (s, 9 H); MS (ESI) m/z: 590.03 (M+H⁺).

### EXAMPLE 7

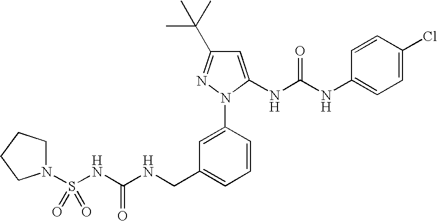

To a stirred solution of chlorosulfonyl isocyanate (19.8 μΛ, 0.227 μμoλ) τν XH₁Xλ₁ (0.5 μΛ) ατ 0° C., was added Example J (97.3 mg, 0.25 mmol) at such a rate that the reaction solution temperature did not rise above 5° C. After being stirred for 1.5 h, a solution of pyrrolidine (18.8 μL, 0.227 mmol) and Et₃N (95 μL, 0.678 mmol) in CH₂Cl₂ (1.5 mL) was added at such a rate that the reaction temperature didn rise above 5° C. When addition was completed, the reaction solution was warmed to RT and stirred overnight. The reaction mixture was poured into 10% HCl, extracted with CH₂Cl₂, the organic layer was washed with saturated NaCl, dried over Mg₂SO₄, and filtered. After removal of the solvents, the crude product was purified by preparative HPLC to yield 1-(3-tert-butyl-1-[[3-N-[[(1-pyrrolidinylsulphonyl)amino]carbonyl]aminomethyl]phenyl]-1H-pyrazol-5-yl)-3-(4-chlorophenyl)urea. ¹HNMR (CDCl₃): δ 7.38 (m, 1 H), 7.36-7.42 (m, 3 H), 7.23 (d, J=8.8 Hz, 2 H), 7.40 (d, J=8.8 Hz, 2 H), 6.43 (s, 1 H), 4.59 (s, 1 H), 4.43 (s, 2 H); 1.81 (s, 2 H), 1.33 (s, 9 H); MS (ESI) m/z: 574.10 (M+H⁺).

### EXAMPLE 8

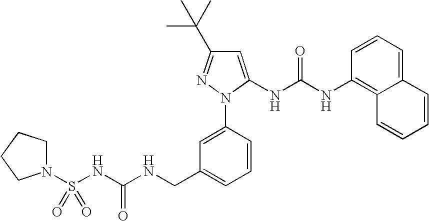

The title compound was made in a manner analogous to Example 7 utilizing Example I to yield 1-(3-tert-butyl-1-[[3-N-[[(1-pyrrolidinylsulphonyl)amino]carbonyl]aminomethyl]-phenyl]-1H-pyrazol-5-yl)-3-(naphthalen-1-yl)urea. ¹HNMR (CDCl₃): δ 7.88 (m, 2 H), 7.02-7.39 (m, 2 H), 7.43-7.50 (m, 7 H), 6.48 (s, 1 H), 4.45 (s, 1 H), 3.32-3.36 (m, 4 H), 1.77-1.81 (m, 4 H), 1.34 (s,9 H); MS (ESI) m/z: 590.03 (M+H⁺).

### EXAMPLE 9

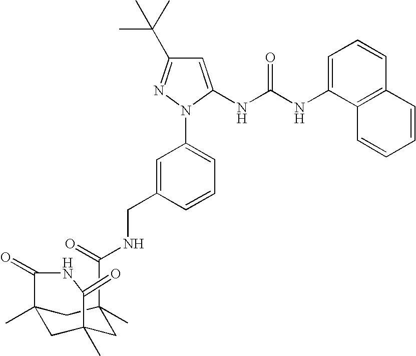

To a solution of Reagent BB (36 mg, 0.15 mmol), Example 1 (62 mg, 0.15 mmol), HOBt (40 mg, 0.4 mmol) and NMM (0.1 mL, 0.9 mmol) in DMF (10 mL) was added EDCI (58 mg, 0.3 mmol). After being stirred overnight, the mixture was poured into water (15 mL) and extracted with EtOAc (35 mL). The organic layers were combined, washed with brine, dried with Na₂SO₄, and concentrated in vacuo. The residue was purified by preparative TLC to yield 1,5,7-trimethyl-2,4-dioxo-3-azabicyclo[3.3.1]nonane-7-carboxylic acid 3-[3-t-butyl-5-(3-naphthalen-1-yl-ureido)-pyrazol-1-yl]benzylamide (22 mg). ¹H NMR (CDCl₃): δ 8.40 (s, 1H), 8.14 (d, J=8.0 Hz, 2H), 7.91 (s, 1H), 7.87 (s, 1H), 7.86 (d, J=7.2 Hz, 1H), 7.78 (d, J=7.6 Hz, 1H), 7.73 (d, J=8.4 Hz, 1H), 7.69 (d, J=8.4 Hz, 1H), 7.57-7.40 (m, 4H), 7.34 (d, J=7.6 Hz, 1H), 6.69 (s, 1H), 6.32 (t, J=5.6 Hz, 1H), 5.92 (brs, 1H), 4.31 (d, J=5.6 Hz, 2H), 2.37 (d, J=14.8 Hz, 2H), 1.80 (d, J=13.2 Hz, 1H), 1.35 (s, 9H), 1.21 (d, J=13.2 Hz, 1H), 1.15 (s, 3H), 1.12 (d, J=12.8 Hz, 2H), 1.04 (s, 6H); MS (ESI) m/z: 635 (M+H⁺).

### EXAMPLE 10

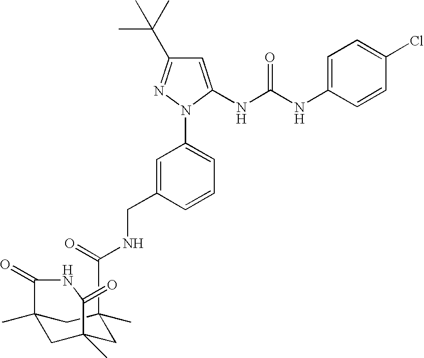

The title compound, was synthesized in a manner analogous to Example 9 utilizing Example J to yield 1,5,7-trimethyl-2,4-dioxo-3-aza-bicyclo[3.3.1]nonane-7-carboxylic acid 3-{3-t-butyl-5-[3-(4-chloro-phenyl)-ureido]-pyrazol-1-yl}benzylamide. ¹H NMR (CDCl₃): δ 8.48 (s, 1H), 7.78 (s, 1H), 7.75 (d, J=8.0 Hz, 1H), 7.69 (s, 1H), 7.53 (t, J=8.0 Hz, 1H), 7.48 (d, J=8.8 Hz, 2H), 7.26 (m, 3H), 6.62 (s, 1H), 6.35(t, J=6.0 Hz, 1H), 5.69 (brs, 1H), 4.26 (d, J=6.0 Hz, 2H), 2.48 (d, J=14.0 Hz, 2H), 1.87 (d, J=13.6 Hz, 1H), 1.35 (s, 9H), 1.25 (m, 6H), 1.15 (s, 6H); MS (ESI) m/z: 619 (M+H⁺).

### EXAMPLE 11

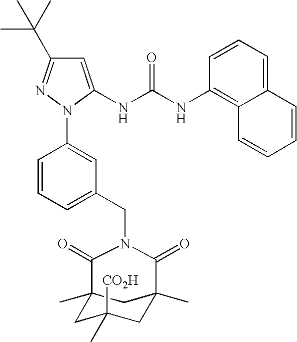

A mixture of Example I (41 mg, 0.1 mmol), Kemp acid anhydride (24 mg, 0.1 mmol) and Et₃N (100 mg, 1 mmol) in anhydrous CH₂Cl₂ (2 mL) were stirred overnight at RT, and concentrated in vacuo. Anhydrous benzene (20 mL) was added to the residue, the mixture was refluxed for 3 h, concentrated in vacuo and purified by preparative HPLC to yield 3-{3-[3-t-butyl-5-(3-naphthalen-1-yl-ureido)-pyrazol-1-yl]-benzyl}-1,5-di-methyl-2,4-dioxo-3-aza-bicyclo[3.3.1]nonane-7-carboxylic acid (8.8 mg, 14%). ¹H NMR (CD₃OD): δ 7.3-7.4 (m, 2H), 7.20 (m, 2H), 7.4-7.6 (m, 7H), 6.50 (m, 1H), 4.80 (s, 2H), 2.60 (d, J=14 Hz, 2H), 1.90 (m, 1H), 1.40 (m, 1H), 1.30 (m, 2H), 1.20 (s, 3H), 1.15 (s, 6H); MS (ESI) m/z: 636 (M+H⁺).

### EXAMPLE 12

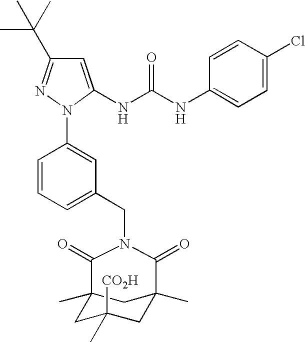

The title compound, was synthesized in a manner analogous to Example 11 utilizing Example J to yield 3-{3-[3-t-butyl-5-(3-naphthalen-1-yl-ureido)-pyrazol-1-yl]-benzyl}-1,5-dimethyl-2,4-dioxo-3-aza-bicyclo[3.3.1]nonane-7-carboxylic acid. ¹H NMR (CD₃OD): δ 7.2-7.5 (m, 7H), 6.40 (s. 1H), 4.70 (s, 2H), 2.60 (d, J=14 Hz, 2H), 1.90 (m, 1H), 1.50 (m, 1H), 1.45 (s, 9H), 1.30 (m, 2H), 1.21 (s, 3H), 1.18 (s, 6H); MS (ESI) m/z: 620 (M+H⁺).

### EXAMPLE 13

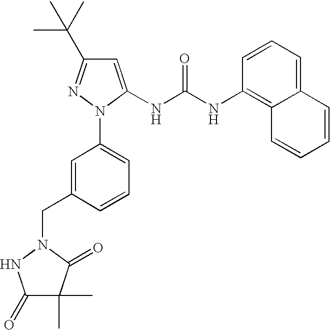

The title compound was synthesized in a manner analogous to Example 1 utilizing Example E and 4,4-dimethyl-3,5-dioxo-pyrazolidine to yield 1-(3-tert-butyl-1-{3-[(4,4-dimethyl-3,5-dioxopyrazolidin-1-yl)methyl]phenyl}-H-pyrazol-5-yl)-3-(naphthalen-1-yl)urea. ¹H NMR (CD₃OD): δ 7.88-7.86 (m, 2H), 7.71-7.68 (m, 2H), 7.58 (m, 2H), 7.60-7.42 (m, 5H), 6.49 (s, 1H), 4.85 (s, 1H), 1.34 (s, 9H), 1.27 (s, 6H); MS (ESI) m/z: 525 (M+H⁺).

### EXAMPLE 14

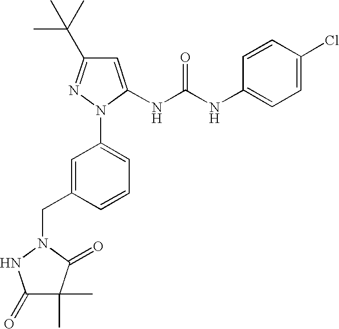

The title compound was synthesized in a manner analogous to Example 1 utilizing Example G and 4,4-dimethyl-3,5-dioxo-pyrazolidine to yield 1-(3-tert-butyl-1-{3-[(4,4-dimethyl-3,5-dioxopyrazolidin-1-yl)methyl]phenyl}-1H-pyrazol-5-yl)-3-(4-chlorophenyl)urea. ¹H NMR (CD₃OD): δ 7.60-7.20 (m, 8H), 6.43 (s, 1H), 4.70 (s, 1H), 1.34 (s, 9H), 1.26 (s, 6H); MS (ESI) m/z: 509, 511 (M+H⁺).

### EXAMPLE 15

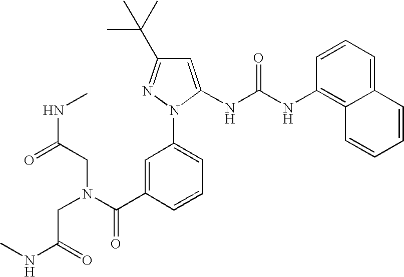

Example B was saponified with 2N LiOH in MeOH, and to the resulting acid (64.2 mg, 0.15 mmol) were added HOBt (30 mg, 0.225 mmol), Example K (24 mg, 0.15 mmol) and 4-methylmorpholine (60 mg, 0.60 mmol 4.0 equiv), DMF (3 mL) and EDCI (43 mg, 0.225 mmol). The reaction mixture was stirred at RT overnight and poured into H₂O (3 mL), and a white precipitate collected and further purified by preparative HPLC to yield 1-[1-(3 {bis[(methylcarbamoyl)methyl]carbamoyl}phenyl)-3-tert-butyl-1H-pyrazol-5-yl]-3-(naphthalen-1-yl)urea (40 mg). ¹H NMR (CDCl₃): δ 8.45 (brs, 1H), 8.10 (d, J=7.6 Hz, 1H), 7.86-7:80 (m, 2H), 7.63-7.56 (m, 2H), 7.52 (s, 1H), 7.47-7.38 (m, 3H), 7.36-7.34 (m, 1H), 7.26 (s, 1H), 7.19-7.17 (m, 2H), 6.60 (s, 1H), 3.98 (s, 2H), 3.81 (s, 3H), 2.87 (s, 3H), 2.63 (s, 3H), 1.34 (s, 9H); MS (ESI) m/z: 570 (M+H⁺).

### EXAMPLE 16

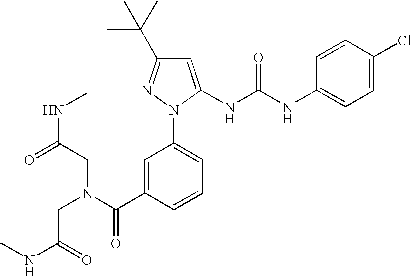

The title compound was synthesized in a manner analogous to Example 15 utilizing Example C (37 mg) and Example K to yield 1-[1-(3-{bis[(methylcarbamoyl)methyl]carbamoyl}phenyl)-3-tert-butyl-1H-pyrazol-5-yl]-3-(4-chlorophenyl)urea. ¹H NMR (CD₃OD): δ 8.58 (brs, 1H), 8.39 (brs, 1H), 7.64-7.62 (m, 3H), 7.53-7.51 (m, 1H), 7.38 (d, J=9.2 Hz, 2H), 7.25 (d, J=8.8 Hz, 2H), 6.44 (s, 1H), 4.17 (s, 2H), 4.11 (s, 2H), 2.79 (s, 3H), 2.69 (s, 3H), 1.34-1.28 (m, 12H); MS (ESI) m/z: 554 (M+H⁺).

### EXAMPLE 17

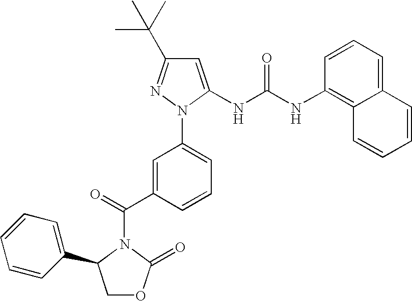

Example B was saponified with 2N LiOH in MeOH, and to the resulting acid (0.642 g, 1.5 mmol) in dry THF (25 mL) at −78° C. were added freshly distilled triethylamine (0.202 g, 2.0 mmol) and pivaloyl chloride (0.216 g, 1.80 mmol) with vigorous stirring. After stirring at −78° C. for 15 min and at 0° C. for 45 min, the mixture was again cooled to −78° C. and then transferred into the THF solution of lithium salt of D-4-phenyl-oxazolidin-2-one [*: The lithium salt of the oxazolidinone regeant was previously prepared by the slow addition of n-BuLi (2.50M in hexane, 1.20 mL, 3.0 mmol) into THF solution of D-4-phenyl-oxazoldin-2-one at −78° C.]. The reaction solution was stirred at −78° C. for 2 h and RT overnight, and then quenched with aq. ammonium chloride and extracted with dichloromethane (100 mL). The combined organic layers were dried (Na₂SO₄) and concentrated in vacuo. The residue was purified by preparative HPLC to yield D-1-{5-tert-butyl-2-[3-(2-oxo-4-phenyl-oxazolidinyl-3-carbonyl)phenyl]-2H-pyrazol-3-yl}-3-(naphthalen-1-yl)urea (207 mg, 24%). ¹H NMR (CDCl₃): δ 8.14-8.09 (m, 2H), 8.06 (s, 1H), 7.86-7.81 (m, 4H), 7.79 (s, 1H), 7.68-7.61 (m, 2H), 7.51-7.40 (m, 9H), 6.75 (s, 1H), 5.80 (t, J=9.2, 7.6 Hz, 1H), 4.89 (t, J=9.2 Hz, 1H), 4.42 (dd, J=9.2, 7.6 Hz, 1H), 1.37 (s, 9H); MS (ESI) m/z: 574 (M+H⁺).

### EXAMPLE 18

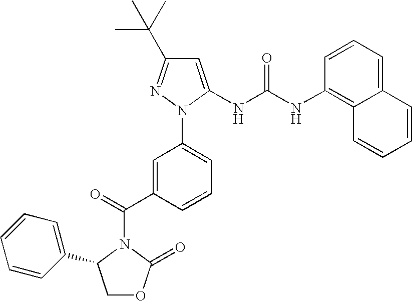

The title compound was synthesized in a manner analogous to Example 17 utilizing Example B and L-4-phenyl-oxazolidin-2-one to yield L-1-{5-tert-butyl-2-[3-(2-oxo-4-phenyl-oxazolidinyl-3-carbonyl)phenyl]-2H-pyrazol-3-yl}-3-(naphthalen-1-yl)urea ¹H NMR (CDCl₃): δ 8.14-8.09 (m, 2H), 8.06 (s, 1H), 7.86-7.81 (m, 4H), 7.79 (s, 1H), 7.68-7.61 (m, 2H), 7.51-7.40 (m, 9H), 6.75 (s, 1H), 5.80 (t, J=9.2, 7.6 Hz, 1H), 4.89 (t, J=9.2 Hz, 1H), 4.42 (dd, J=9.2, 7.6 Hz, 1H), 1.37 (s, 9H); MS (ESI) m/z: 574 (M+H⁺)

### EXAMPLE 19

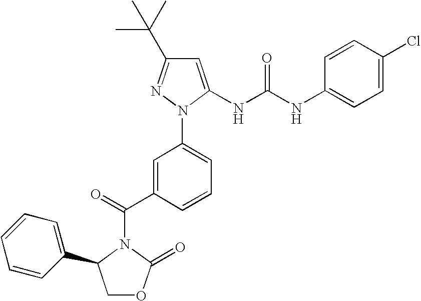

The title compound was synthesized in a manner analogous to Example 17 utilizing Example C and D-4-phenyl-oxazolidin-2-one to yield D-1-{5-tert-butyl-2-[3-(2-oxo-4-phenyl-oxazolidinyl-3-carbonyl)phenyl]-2H-pyrazol-3-yl}-3-(4-chlorophenyl)urea. ¹H NMR (CDCl₃): δ 7.91 (s, 1H), 7.85 (d, J=8.0 Hz, 1H), 7.79 (d, J=7.6 Hz, 1H), 7.71 (m, 1H), 7.65 (m, 1H), 7.49-7.40 (m, 8H), 7.26-7.24 (m, 2H), 6.68 (s, 1H), 5.77 (dd, J=8.8, 8.0 Hz, 1H), 4.96 (t, 8.8 Hz, 1H), 4.44 (dd, J=8.8, 8.0 Hz, 1H), 1.36 (s, 9H); MS (ESI) m/z: 558 (M+H⁺)

### EXAMPLE 20

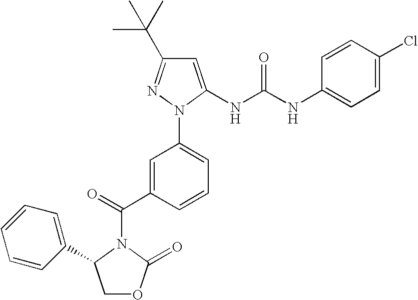

The title compound was synthesized in a manner analogous to Example 17 utilizing Example C and L-4-phenyl-oxazolidin-2-one to yield L-1-{5-tert-butyl-2-[3-(2-oxo-4-phenyl-oxazolidinyl-3-carbonyl)phenyl]-2H-pyrazol-3-yl}-3-(4-chlorophenyl)urea. ¹H NMR (CDCl₃): δ 7.91 (s, 1H), 7.85 (d, J=8.0 Hz, 1H), 7.79 (d, J=7.6 Hz, 1H), 7.71 (m, 1H), 7.65 (m, 1H), 7.49-7.40 (m, 8H), 7.26-7.24 (m, 2H), 6.68 (s, 1H), 5.77 (dd, J=8.8, 8.0 Hz, 1H), 4.96 (t, 8.8 Hz, 1H), 4.44 (dd, J=8.8, 8.0 Hz, 1H), 1.36 (s, 9H); MS (ESI) m/z: 558 (M+H⁺)

### EXAMPLE L

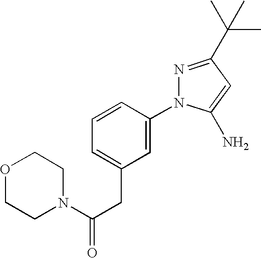

To a stirred suspension of (3-nitro-phenyl)-acetic acid (2 g) in CH₂Cl₂ (40 ml, with a catalytic amount of DMF) at 0° C. under N₂ was added oxalyl chloride (1.1 ml) drop wise. The reaction mixture was stirred for 40 min morpholine (2.5 g) was added. After stirring for 20 min, the reaction mixture was filtered. The filtrate was concentrated in vacuo to yield 1-morpholin-4-yl-2-(3-nitro-pheny)-ethanone as a solid (2 g). A mixture of 1-morpholin-4-yl-2-(3-nitro-pheny)-ethanone (2 g) and 10% Pd on activated carbon (0.2 g) in ethanol (30 ml) was hydrogenated at 30 psi for 3 h and filtered over Celite. Removal of the volatiles in vacuo provided 2-(3-amino-phenyl)-1-morpholin-4-yl-ethanone (1.7 g). A solution of 2-(3-amino-phenyl)-1-morpholin-4-yl-ethanone (1.7 g, 7.7 mmol) was dissolved in 6 N HCl (15 ml), cooled to 0° C., and vigorously stirred. Sodium nitrite (0.54 g) in water (8 ml) was added. After 30 min, tin (II) chloride dihydrate (10 g) in 6 N HCl (30 ml) was added. The reaction mixture was stirred at 0° C. for 3 h. The pH was adjusted to pH 14 with solid potassium hydroxide and extracted with EtOAc. The combined organic extracts were concentrated in vacuo provided 2-(3-hydrazin-phenyl)-1-morpholin-4-yl-ethanone (1.5 g). 2-(3-Hydrazinophenyl)-1-morpholin-4-yl-ethanone (3 g) and 4,4-dimethyl-3-oxopentanenitrile (1.9 g, 15 mmol) in ethanol (60 ml) and 6 N HCl (1 ml) were refluxed for 1 h and cooled to RT. The reaction mixture was neutralized by adding solid sodium hydrogen carbonate. The slurry was filtered and removal of the volatiles in vacuo provided a residue that was extracted with ethyl acetate. The volatiles were removed in vacuo to provide 2-[3-(3-tert-butyl-5-amino-H-pyrazol-1-yl)phenyl]-1-morpholinoethanone (4 g), which was used without further purification.

### EXAMPLE 21

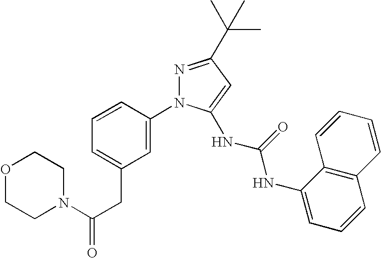

A mixture of Example L (0.2 g, 0.58 mmol) and 1-naphthylisocyanate (0.10 g, 0.6 mmol) in dry CH₂Cl₂ (4 ml) was stirred at RT under N₂ for 18 h. The solvent was removed in vacuo and the crude product was purified by column chromatography using ethyl acetate/hexane/CH₂Cl₂ (3/1/0.7) as the eluent (0.11 g, off-white solid) to yield 1-{3-tert-butyl-1-[3-(2-morpholino-2-oxoethyl)phenyl]-1H-pyrazol-5-yl}-3-(naphthalene-1-yl)urea. mp: 194-196; ¹H NMR (200 MHz, DMSO-d₆): δ 9.07 (1H, s), 8.45 (s, 1H), 8.06-7.93 (m, 3H), 7.69-7.44 (m, 7H), 7.33-7.29 (d, 6.9 Hz, 1H), 6.44 (s, 1H), 3.85 (m, 2H), 3.54-3.45 (m, 8H), 1.31 (s, 9H); MS:

### EXAMPLE 22

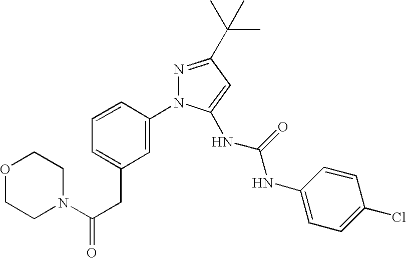

The title compound was synthesized in a manner analogous to Example 21 utilizing Example L (0.2 g, 0.58 mmol) and 4-chlorophenylisocyanate (0.09 g, 0.6 mmol) to yield 1-{3-tert-butyl-1-[3-(2-morpholino-2-oxoethyl)phenyl]-1H-pyrazol-5-yl}-3-(4-chlorophenyl)urea. mp: 100 104; ¹H NMR (200 MHz, DMSO-d₆): δ 9.16 (s, 1H), 8.45 (s, 1H), 7.52-7.30 (in, 8H), 6.38 (s, 1H), 3.83 (in, 1H), 3.53-3.46 (in, 8H), 1.30 (s, 9H); MS:

### EXAMPLE 23

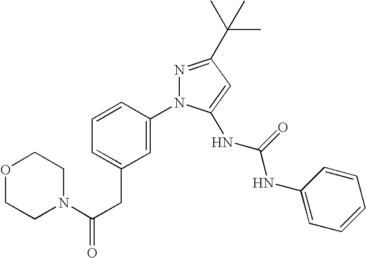

The title compound is synthesized in a manner analogous to Example 21 utilizing Example L (0.2 g, 0.58 mmol) and phenylisocyanate (0.09 g, 0.6 mmol) to yield 1-{3-tert-butyl-1-[3-(2-morpholino-2-oxoethyl)phenyl]-1H-pyrazol-5-yl}-3-phenylurea.

### EXAMPLE 24

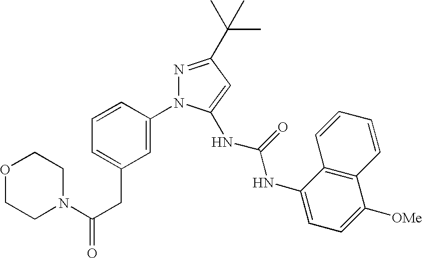

The title compound is synthesized in a manner analogous to Example 21 utilizing Example L (0.2 g, 0.58 mmol) and 1-isocyanato-4-methoxy-naphthalene to yield 1-{3-tert-butyl-1-[3-(2-morpholino-2-oxoethyl)phenyl]-1H-pyrazol-5-yl}-3-(1-methoxynaphthalen-4-yl)urea.

### EXAMPLE M

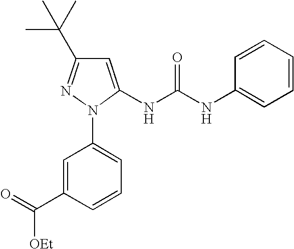

The title compound is synthesized in a manner analogous to Example C utilizing Example A and phenylisocyanate to yield ethyl 3-(3-tert-butyl-5-(3-phenylureido)-1H-pyrazol-1-yl)benzoate.

### EXAMPLE N

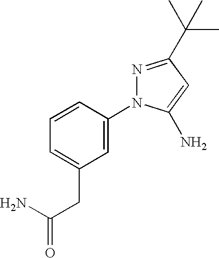

A solution of (3-nitrophenyl)acetic acid (23 g, 127 mmol) in methanol (250 ml) and a catalytic amount of concentrated in vacuo H₂SO₄ was heated to reflux for 18 h. The reaction mixture was concentrated in vacuo to a yellow oil. This was dissolved in methanol (250 ml) and stirred for 18 h in an ice bath, whereupon a slow flow of ammonia Was charged into the solution. The volatiles were removed in vacuo. The residue was washed with diethyl ether and dried to afford 2-(3-nitrophenyl)acetamide (14 g, off-white solid). ¹H NMR (CDCl₃): δ 8.1 (s, 1H), 8.0 (d, 1H), 7.7 (d, 1H), 7.5 (m, 1H), 7.1 (bd s, 1H), 6.2 (brs, 1H), 3.6 (s, 2H).

The crude material from the previous reaction (8 g) and 10% Pd on activated carbon (1 g) in ethanol (100 ml) was hydrogenated at 30 psi for 18 h and filtered over Celite. Removal of the volatiles in vacuo provided 2-(3-aminophenyl)acetamide (5.7 g). A solution of this material (7 g, 46.7 mmol) was dissolved in 6 N HCl (100 ml), cooled to 0° C., and vigorously stirred. Sodium nitrite (3.22 g, 46.7 mmol) in water (50 ml) was added. After 30 min, tin (II) chloride dihydrate (26 g) in 6 N HCl (100 ml) was added. The reaction mixture was stirred at 0° C. for 3 h. The pH was adjusted to pH 14 with 50% aqueous NaOH solution and extracted with ethyl acetate. The combined organic extracts were concentrated in vacuo provided 2-(3-hydrazinophenyl)acetamide.

The crude material from the previous reaction (ca. 15 mmol) and 4,4-dimethyl-3-oxopentanenitrile (1.85 g, 15 mmol) in ethanol (60 ml) and 6 N HCl (1.5 ml) was refluxed for 1 h and cooled to RT. The reaction mixture was neutralized by adding solid sodium hydrogen carbonate. The slurry was filtered and removal of the volatiles in vacuo provided a residue, which was extracted with ethyl acetate. The solvent was removed in vacuo to provide 2-[3-(3-tert-butyl-5-amino-1H-pyrazol-1-yl)phenyl]acetamide as a white solid (3.2 g), which was used without further purification.

### EXAMPLE 25

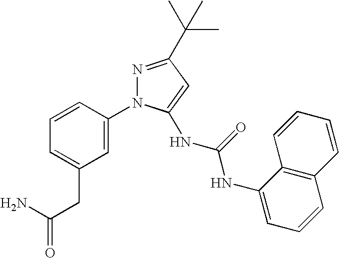

A mixture of Example N (2 g, 0.73 mmol) and 1-naphthylisocyanate (0.124 g, 0.73 mmol) in dry CH₂Cl₂ (4 ml) was stirred at RT under N₂ for 18 h. The solvent was removed in vacuo and the crude product was washed with ethyl acetate (8 ml) and dried in vacuo to yield 1-{3-tert-butyl-1-[3-(carbamoylmethyl)phenyl)-1H-pyrazol-5-yl}-3-(naphthalene-1-yl)urea as a white solid (0.22 g). mp: 230 (dec.); ¹H NMR (200 MHz, DMSO-d₆); δ9.12 (s, 1H), 8.92 (s, 1H), 8.32-8.08 (m, 3H), 7.94-7.44 (m, 8H), 6.44 (s, 1H), 3.51 (s, 2H), 1.31 (s, 9H); MS:

### EXAMPLE 26

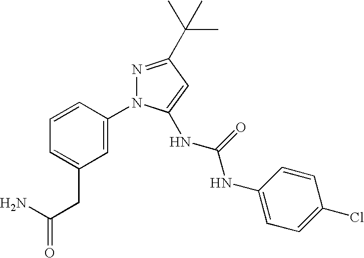

The title compound was synthesized in a manner analogous to Example 23 utilizing Example N (0.2 g, 0.73 mmol) and 4-chlorophenylisocyanate (0.112 g, 0.73 mmol) to yield 1-{3-tert-butyl-1-[3-(carbamoylmethyl)phenyl)-1H-pyrazol-5-yl}-3-(4-chlorophenyl)urea as a white solid (0.28 g). mp: 222 224. (dec.); ¹H NMR (200 MHz, DMSO-d₆); δ9.15 (s, 1H), 8.46 (s, 1H), 7.55-7.31 (m, 8H), 6.39 (s, 1H), 3.48 (s, 2H), 1.30 (s, 9H); MS:

### EXAMPLE O

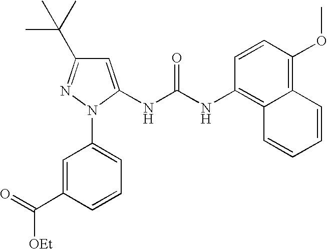

The title compound is synthesized in a manner analogous to Example C utilizing Example A and 1-isocyanato-4-methoxy-naphthaleneto yield ethyl 3-(3-tert-butyl-5-(3-(1-methoxynaphthalen-4-yl)ureido)-1H-pyrazol-1-yl)benzoate.

### EXAMPLE 27

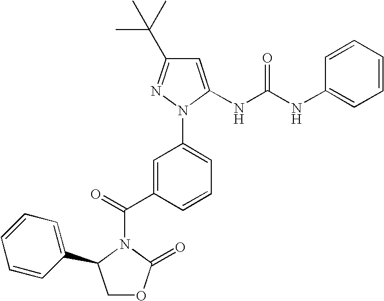

The title compound is synthesized in a manner analogous to Example 17 utilizing Example M and D-4-phenyl-oxazolidin-2-one to yield D-1-{5-tert-butyl-2-[3-(2-oxo-4-phenyl-oxazolidinyl-3-carbonyl)phenyl]-2H-pyrazol-3-yl}-3-phenylurea.

### EXAMPLE 28

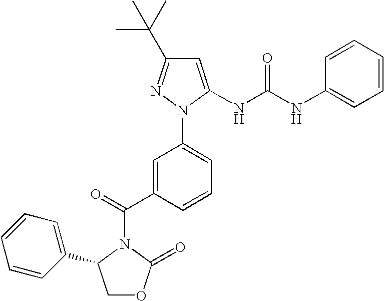

The title compound is synthesized in a manner analogous to Example 17 utilizing Example M and and L-4-phenyl-oxazolidin-2-one to yield L-1-{5-tert-butyl-2-[3-(2-oxo-4-phenyl-oxazolidinyl-3-carbonyl)phenyl]-2H-pyrazol-3-yl}-3-phenylurea.

### EXAMPLE P

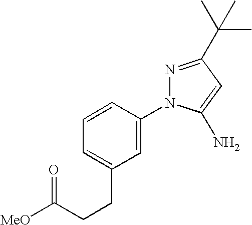

A mixture of 3-(3-amino-phenyl)-acrylic acid methyl ester (6 g) and 10% Pd on activated carbon (1 g) in ethanol (50 ml) was hydrogenated at 30 psi for 18 h and filtered over Celite. Removal of the volatiles in vacuo provided 3-(3-amino-phenyl)propionic acid methyl ester (6 g).

A vigorously stirred solution of the crude material from the previous reaction (5.7 g, 31.8 mmol) dissolved in 6 N HCl (35 ml) was cooled to 0° C., and sodium nitrite (2.2 g) in water (20 ml) was added. After 1 h, tin (II) chloride dihydrate (18 g) in 6 N HCl (35 ml) was added. And the mixture was stirred at 0° C. for 3 h. The pH was adjusted to pH 14 with solid KOH and extracted with EtOAc. The combined organic extracts were concentrated in vacuo provided methyl 3-(3-hydrazino-phenyl)propionate (1.7 g).

A stirred solution of the crude material from the previous reaction (1.7 g, 8.8 mmol) and 4,4-dimethyl-3-oxopentanenitrile (1.2 g, 9.7 mmol) in ethanol (30 ml) and 6 N HCl (2 ml) was refluxed for 18 h and cooled to RT. The volatiles were removed in vacuo and the residue dissolved in EtOAc and washed with 1 N aqueous NaOH. The organic layer was dried (Na₂SO₄) and concentrated in vacuo and the residue was purified by column chromatography using 30% ethyl acetate in hexane as the eluent to provide methyl 3-[3-(3-tert-butyl-5-amino-1H-pyrazol-1-yl)phenyl]propionate (3.2 g), which was used without further purification

### EXAMPLE 29

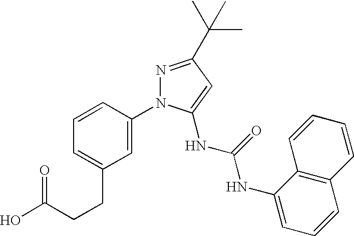

A mixture of Example P (0.35 g, 1.1 mmol) and 1-naphthylisocyanate (0.19 g, 1.05 mmol) in dry CH₂Cl₂ (5 ml) was stirred at RT under N₂ for 20 h. The solvent was removed in vacuo and the residue was stirred in a solution of THF (3 ml)/MeOH (2 ml)/water (1.5 ml) containing lithium hydroxide (0.1 g) for 3 h at RT, and subsequently diluted with EtOAc and dilute citric acid solution. The organic layer was dried (Na₂SO₄), and the volatiles removed in vacuo. The residue was purified by column chromatography using 3% methanol in CH₂Cl₂ as the eluent to yield 3-(3-{3-tert-butyl-5-[3-(naphthalen-1-yl)ureido]-1H-pyrazol-1-yl)phenylpropionic acid (0.22 g, brownish solid). mp: 105-107; ¹H NMR (200 MHz, CDCl₃): δ 7.87-7.36 (m, 10H), 7.18-7.16 (m, 1H), 6.52 (s, 1H), 2.93 (t, J=6.9 Hz, 2H), 2.65 (t, J=7.1 Hz, 2H), 1.37 (s, 9H); MS

### EXAMPLE 30

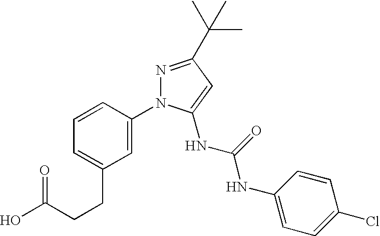

The title compound was synthesized in a manner analogous to Example 29 utilizing Example P (0.30 g, 0.95 mmol) and 4-chlorophenylisocyanate (0.146 g, 0.95 mmol) to yield 3-(3-{3-tert-butyl-5-[3-(4-chloropnehyl)ureido]-1H-pyrazol-1-yl)phenyl)propionic acid (0.05 g, white solid). mp:85 87; ¹H NMR (200 MHz, CDCl₃): δ8.21 (s, 1H), 7.44-7.14 (m, 7H), 6.98 (s, 1H), 6.55 (s, 1H), 2.98 (t, J=5.2 Hz, 2H), 2.66 (t, J=5.6 Hz, 2H), 1.40 (s, 9H); MS

### EXAMPLE Q

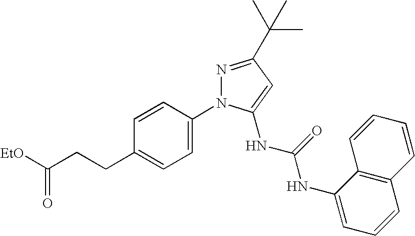

A mixture of ethyl 3-(4-aminophenyl)acrylate(1.5 g) and 10% Pd on activated carbon (0.3 g) in ethanol (20 ml) was hydrogenated at 30 psi for 18 h and filtered over Celite. Removal of the volatiles in vacuo provided ethyl 3-(4-aminophenyl)propionate (1.5 g).

A solution of the crude material from the previous reaction (1.5 g, 8.4 mmol) was dissolved in 6 N HCl (9 ml), cooled to 0° C., and vigorously stirred. Sodium nitrite (0.58 g) in water (7 ml) was added. After 1 h, tin (II) chloride dihydrate (5 g) in 6 N HCl (10 ml) was added. The reaction mixture was stirred at 0° C. for 3 h. The pH was adjusted to pH 14 with solid KOH and extracted with EtOAc. The combined organic extracts were concentrated in vacuo provided ethyl 3-(4-hydrazino-phenyl)-propionate(1 g).

The crude material from the previous reaction (1 g, 8.8 mmol) and 4,4-dimethyl-3-oxopentanenitrile (0.7 g) in ethanol (8 ml) and 6 N HCl (1 ml) was refluxed for 18 h and cooled to RT. The volatiles were removed in vacuo. The residue was dissolved in ethyl acetate and washed with 1 N aqueous sodium hydroxide solution. The organic layer was dried (Na₂SO₄) and concentrated in vacuo. The residue was purified by column chromatography using 0.7% methanol in CH₂Cl₂ as the eluent to provide ethyl 3-{4-[3-tert-butyl-5-(3-(naphthalene-1-yl)ureido]-1H-pyrazol-1-yl}phenyl)prpanoate (0.57 g).

### EXAMPLE 31

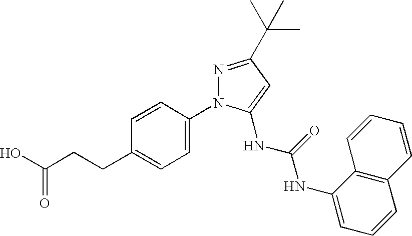

A mixture of Example Q (0.25 g, 0.8 mmol) and 1-naphthylisocyanate (0.13 g, 0.8 mmol) in dry CH₂Cl₂ (5 ml) was stirred at RT under N₂ for 20 h. The solvent was removed in vacuo and the residue was stirred in a solution of THF (3 ml)/MeOH (2 ml)/water (1.5 ml) containing lithium hydroxide (0.1 g) for 3 h at RT and diluted with EtOAc and diluted citric acid solution. The organic layer was dried (Na₂SO₄), and the volatiles removed in vacuo. The residue was purified by column chromatography using 4% methanol in CH₂Cl₂ as the eluent to yield 3-{4-[3-tert-butyl-5-(3-(naphthalene-1-yl)ureido]-1H-pyrazol-1-yl}phenyl)propanonic acid (0.18 g, off-white solid). mp: 120 122; ¹H NMR (200 MHz, CDCl₃): δ 7.89-7.06 (m, 11H), 6.5 (s, 1H), 2.89 (m, 2H), 2.61 (m, 2H), 1.37 (s, 9H); MS

### EXAMPLE 32

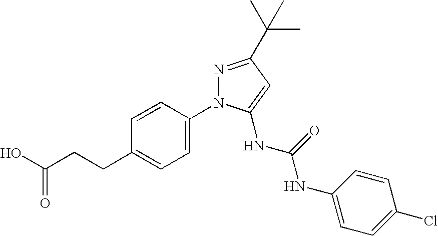

The title compound was synthesized in a manner analogous to Example 31 utilizing Example Q (0.16 g, 0.5 mmol) and 4-chlorophenylisocyanate (0.077 g, 0.5 mmol) to yield 3-{4-[3-tert-butyl-5-(3-(4-chlorphenyl)ureido]-1H-pyrazol-1-yl}phenyl)propanonic acid acid (0.16 g, off-white solid). mp: 112-114; ¹H NMR (200 MHz, CDCl₃): δ 8.16 (s, 1H), 7.56 (s, 1H), 7.21 (s, 2H), 7.09 (s, 2H), 6.42 (s, 1H), 2.80 (m, 2H), 2.56 (m, 2H), 1.32 (s, 9H); MS

### EXAMPLE R

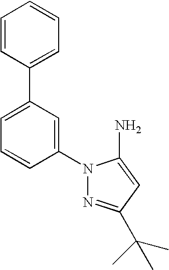

A 250 mL pressure vessel (ACE Glass Teflon screw cap) was charged with 3-nitrobiphenyl (20 g, 0.10 mol) dissolved in THF (˜100 mL) and 10% Pd/C (3 g). The reaction vessel was charged with H₂ (g) and purged three times. The reaction was charged with 40 psi H₂ (g) and placed on a Parr shaker hydrogenation apparatus and allowed to shake overnight at RT. HPLC showed that the reaction was complete thus the reaction mixture was filtered through a bed of Celite and evaporated to yield the amine: 16.7 g (98% yield) In a 250 mL Erlenmeyer flask with a magnetic stir bar, the crude material from the previous reaction (4.40 g, 0.026 mol) was added to 6 N HCl (40 mL) and cooled with an ice bath to ˜⁰° C. A solution of NaNO₂ (2.11 g, 0.0306 mol, 1.18 eq.) in water (5 mL) was added drop wise. After 30 min, SnCl₂.2H₂O (52.0 g, 0.23 mol, 8.86 eq.) in 6N HCl (100 mL) was added and the reaction mixture was allowed to stir for 3 h, then subsequently transferred to a 500 mL round bottom flask. To this, 4,4-dimethyl-3-oxopentanenitrile (3.25 g, 0.026 mol) and EtOH (100 ml) were added and the mixture refluxed for 4 h, concentrated in vacuo and the residue extracted with EtOAc (2×100 mL). The residue was purified by column chromatograph using hexane/EtOAc/Et₃N (8:2:0.2) to yield 0.53 g of Example R. ¹H NMR (CDCl₃): δ 7.5 (m, 18H), 5.8 (s, 1H), 1.3 (s, 9H).

### EXAMPLE 33

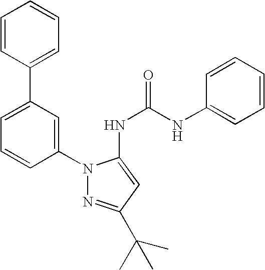

In a dry vial with a magnetic stir bar, Example R (0.145 g; 0.50 mmol) was dissolved in 2 mL CH₂Cl₂ (anhydrous) followed by the addition of phenylisocyanate (0.0544 mL; 0.50 mmol; 1 eq.). The reaction was kept under argon and stirred for 17 h. Evaporation of solvent gave a crystalline mass that was triturated with hexane/EtOAc (4:1) and filtered to yield 1-(3-tert-butyl-1-(3-phenylphenyl)-1H-pyrazol-5-yl)-3-phenylurea (0.185 g, 90%). HPLC purity: 96%; mp: 80 84; ¹H NMR (CDCl₃): δ 7.3 (m, 16H), 6.3 (s, 1H), 1.4 (s, 9H).

### EXAMPLE 34

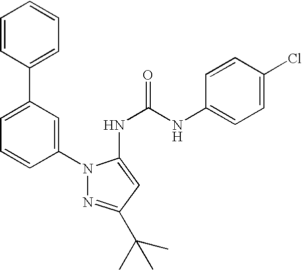

The title compound was synthesized in a manner analogous to Example 33 utilizing Example R (0.145 g; 0.50 mmol) and p-chlorophenylisocyanate (0.0768 g, 0.50 mmol, 1 eq.) to yield 1-(3-tert-butyl-1-(3-phenylphenyl)-1H-pyrazol-5-yl)-3-(4-chlorophenyl)urea (0.205 g, 92%). HPLC purity: 96.5%; mp: 134 136; ¹H NMR (CDCl₃): δ 7.5 (m, 14H), 7.0 (s, 11H), 6.6 (s, 1H), 6.4 (s, 1H), 1.4 (s, 9H).

### EXAMPLE S

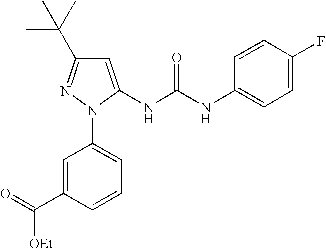

The title compound is synthesized in a manner analogous to Example C utilizing Example A and 4-fluorophenyl isocyanate yield ethyl 3-(3-tert-butyl-5-(3-(4-flurophenyl)ureido)-1H-pyrazol-1-yl)benzoate.

### EXAMPLE 35

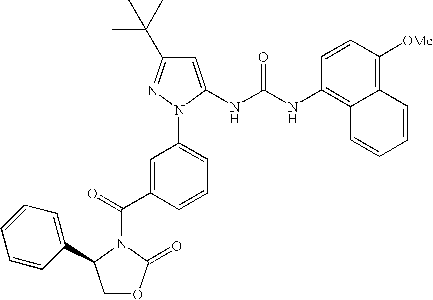

The title compound is synthesized in a manner analogous to Example 17 utilizing Example M and D-4-phenyl-oxazolidin-2-one to yield D-1-{5-tert-butyl-2-[3-(2-oxo-4-phenyl-oxazolidinyl-3-carbonyl)phenyl]-2H-pyrazol-3-yl}-3-(naphthalen-1-yl)urea.

### EXAMPLE 36

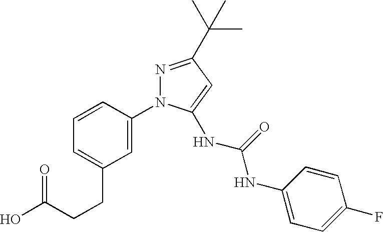

The title compound is synthesized in a manner analogous to Example 29 utilizing Example P (0.30 g, 0.95 mmol) and 4-flu0rophenylisocyanate (0.146 g, 0.95 mmol) to yield 3-(3-(3-tert-butyl-5-(3-(4-fluorophenyl)ureido)-1H-pyrazol-1-yl)phenyl)propanoic acid.

### EXAMPLE T

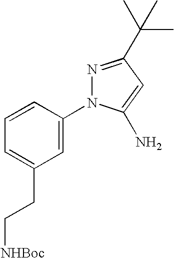

To a stirred solution of Example N (2 g, 7.35 mmol) in THF (6 ml) was added borane-methylsulfide (18 mmol). The mixture was heated to reflux for 90 min and cooled to RT, after which 6 N HCl was added and heated to reflux for 10 min. The mixture was basified with NaOH and extracted with EtOAc. The organic layer was dried (Na₂SO₄) filtered and concentrated in vacuo to yield 3-tert-butyl-1-[3-(2-aminoethyl)phenyl]-1H-pyrazol-5 amine (0.9 g).

A mixture of the crude material from the previous reaction (0.8 g, 3.1 mmol) and di-tert-butylcarbonate (0.7 g, 3.5 mmol) and catalytically amount of DMAP in dry CH₂Cl₂ (5 ml) was stirred at RT under N₂ for 18 h. The reaction mixture was concentrated in vacuo and the residue was purified by column chromatography using 1% methanol in CH₂Cl₂ as the eluent to yield tert-butyl 3-(3-tert-butyl-5-amino-1H-pyrazol-1-yl)phenylcarbamate (0.5 g).

### EXAMPLE 37

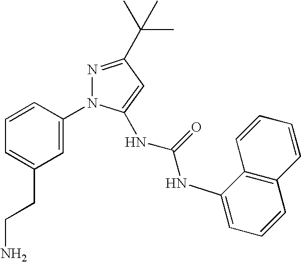

A mixture of Example T (0.26 g, 0.73 mmol) and 1-naphthylisocyanate (0.123 g, 0.73 mmol) in dry CH₂Cl₂ (5 ml) was stirred at RT under N₂ for 48 h. The solvent was removed in vacuo and the residue was purified by column chromatography using 1% methanol in CH₂Cl₂ as the eluent (0.15 g, off-white solid). The solid was then treated with TFA (0.2 ml) for 5 min and diluted with EtOAc. The organic layer was washed with saturated NaHCO₃ solution and brine, dried (Na₂SO₄), filtered and concentrated in vacuo to yield 1-{3-tert-butyl-1-[3-(2-Aminoethyl)phenyl]-1H-pyrazol-5-yl}-3-(naphthalen-1-yl)urea as a solid (80 mg). mp: 110-112; ¹H NMR (200 MHz, DMSO-d₆): δ 9.09 (s, 1H), 8.90 (s, 1H), 8.01-7.34 (m, 11H), 6.43 (s, 1H), 3.11 (m, 2H), 2.96 (m, 2H), 1.29 (s, 9H); MS

### EXAMPLE 38

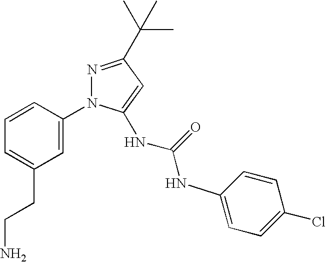

The title compound was synthesized in a manner analogous to Example 37 utilizing Example T (0.15 g, 0.42 mmol) and 4-chlorophenylisocyanate (0.065 g, 0.42 mmol) to yield 1-{3-tert-butyl-1-[3-(2-Aminoethyl)phenyl]-1H-pyrazol-5-yl}-3-(4-chlorophenyl)urea as an off-white solid (20 mg). mp:125-127; ¹H NMR (200 MHz, CDCl₃): δ 8.81 (s, 1H), 8.66 (s, 1H), 7.36-7.13 (m, 8H), 6.54 (s, 1H), 3.15 (brs, 2H), 2.97 (brs, 2H), 1.32 (s, 9H); MS

### EXAMPLE U

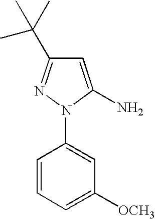

In a 250 mL Erlenmeyer flask with a magnetic stir bar, m-anisidine (9.84 g, 0.052 mol) was added to 6 N HCl (80 mL) and cooled with an ice bath to 0° C. A solution of NaNO₂ (4.22 g, 0.0612 mol, 1.18 eq.) in water (10 mL) was added drop wise. After 30 min, SnCl₂.2H₂O (104.0 g, 0.46 mol, 8.86 eq.) in 6 N HCl (200 mL) was added and the reaction mixture was allowed to stir for 3 h., and then subsequently transferred to a 1000 mL round bottom flask. To this, 4,4-dimethyl-3-oxopentanenitrile (8.00 g, 0.064 mol) and EtOH (200 mL) were added and the mixture refluxed for 4 h, concentrated in vacuo and the residue recrystallized from CH₂Cl₂ to yield 3-tert-butyl-1-(3-methoxyphenyl)-1H-pyrazol-5-amine as the HCl salt (13.9 g).

The crude material from the previous reaction (4.65 g, 0.165 mol) was dissolved in 30 mL of CH₂Cl₂ with Et₃N (2.30 mL, 0.0165 mol, 1 eq.) and stirred for 30 min Extraction with water followed by drying of the organic phase with Na₂SO₄ and concentration in vacuo yielded a brown syrup that was the free base, 3-tert-butyl-1-(3-methoxyphenyl)-1H-pyrazol-5-amine (3.82 g, 94.5%), which was used without further purification.

### EXAMPLE 39

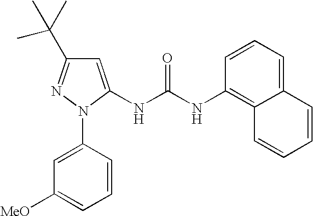

In a dry vial with a magnetic stir bar, Example U (2.62 g, 0.0107 mol) was dissolved in CH₂Cl₂ (5 mL, anhydrous) followed by the addition of 1-naphthylisocyanate (1.53 mL, 0.0107 mol, 1 eq.). The reaction was kept under Ar and stirred for 18 h. Evaporation of solvent followed by column chromatography with EtOAc/hexane/Et₃N (7:2:0.5) as the eluent yielded 1-[3-tert-butyl-1-(3-methoxyphenyl)-1H-pyrazol-5-yl]-3-(naphthalen-1-yl)urea (3.4 g, 77%). HPLC: 97%; mp: 78-80; ¹H NMR (CDCl₃): δ 7.9-6.8 (m, 15H), 6.4 (s, 1H), 3.7 (s, 3H), 1.4 (s, 9H).

### EXAMPLE 40

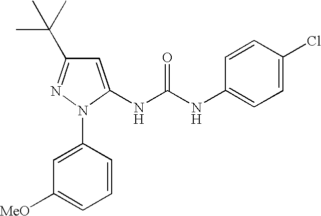

The title compound was synthesized in a manner analogous to Example 39 utilizing Example U (3.82 g; 0.0156 mol) and p-chlorophenylisocyanate (2.39 g, 0.0156 mol, 1 eq.), purified by trituration with hexane/EtOAc (4:1) and filtered to yield 1-[3-tert-butyl-1-(3-methoxyphenyl)-1H-pyrazol-5-yl]-3-(4-chlorophenyl)urea (6.1 g, 98%). HPLC purity: 95%; mp: 158-160; ¹H NMR (CDCl₃): δ 7.7 (s, 1H); δ7.2 6.8 (m, 8H), 6.4 (s, 1H), 3.7 (s, 3H), 1.3 (s, 9H).

### EXAMPLE 41

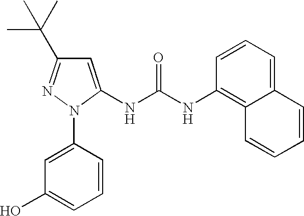

In a 100 ml round bottom flask equipped with a magnetic stir bar, Example 39 (2.07 g) was dissolved in CH₂Cl₂ (20 mL) and cooled to 0° C. with an ice bath. BBr₃ (1 M in CH₂Cl₂; 7.5 mL) was added slowly. The reaction mixture was allowed to warm warm to RT overnight. Additional BBr₃ (1 M in CH₂Cl₂, 2×1 mL, 9.5 mmol total added) was added and the reaction was quenched by the addition of MeOH. Evaporation of solvent led to a crystalline material that was chromatographed on silica gel (30 g) using CH₂Cl₂/MeOH (9.6:0.4) as the eluent to yield 1-[3-tert-butyl-1-(3-hydroxyphenyl)-1H-pyrazol-5-yl]-3-(naphthalene-1-yl)urea (0.40 g, 20%). ¹H NMR (DMSO-d₆): δ 9.0 (s, 1H), 8.8 (s, 1H), 8.1-6.8 (m, 11H), 6.4 (s, 1H), 1.3 (s, 9H). MS (ESI) m/z: 401 (M+H⁺).

### EXAMPLE 42

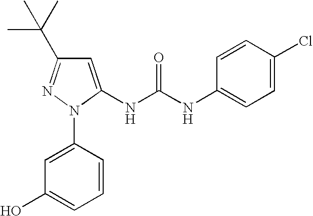

The title compound was synthesized in a manner analogous to Example 41 utilizing Example 40 (2.00 g, 5 mmol) that resulted in a crystalline material that was filtered and washed with MeOH to yield 1-[3-tert-butyl-1-(3-hydroxyphenyl)-1H-pyrazol-5-yl]-3-(4-chlorophenyl)urea (1.14 g, 60%). HPLC purity: 96%; mp: 214-216; ¹H NMR (CDCl₃): δ 8.4 (s, 1H), 7.7 (s, 1H), 7.4-6.6 (m, 9H), 1.3 (s, 9H).

### EXAMPLE V

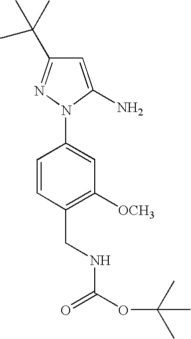

The starting material, 1-[4-(aminomethyl)phenyl]-3-tert-butyl-N-nitroso-1H-pyrazol-5-amine, was synthesized in a manner analogous to Example A utilizing 4-aminobenzamide and 4,4-dimethyl-3-oxopentanenitrile.

A 1 L four-necked round bottom flask was equipped with a stir bar, a source of dry Ar, a heating mantle, and a reflux condenser. The flask was flushed with Ar and charged with the crude material from the previous reaction (12 g, 46.5 mmol; 258.1 g/mol) and anhydrous THF (500 ml). This solution was treated cautiously with LiAlH₄ (2.65 g, 69.8 mmol) and the reaction was stirred overnight. The reaction was heated to reflux and additional LiAlH₄ was added complete (a total of 8.35 g added). The reaction was cooled to 0 and H₂O (8.4 ml), 15% NaOH (8.4 ml) and H₂O (24 ml) were added sequentially; The mixture was stirred for 2 h, the solids filtered through Celite, and washed extensively with THF, the solution was concentrated in vacuo to yield 1-(4-(aminomethyl-3-methoxy)phenyl)-3-tert-butyl-1H-pyrazol-5-amine (6.8 g) as an oil.

A 40 mL vial was equipped with a stir bar, a septum, and a source of Ar. The vial was charged with the crude material from the previous reaction (2 g, 8.2 mmol, 244.17 g/mol) and CHCl₃ (15 mL) were cooled to 0 under Ar and di-tert-butylcarbonate (1.9 g, 9.0 mmol) dissolved in CHCl₃ (5 mL) was added drop wise over a 2 min period. The mixture was treated with 1N KOH (2 mL), added over a 2 h period. The resulting emulsion was broken with the addition of saturated NaCl solution, the layers were separated and the aqueous phase extracted with CH₂Cl₂ (2×1.5 ml). The combined organic phases were dried over Na₂SO4, filtered, concentrated in vacuo to yield tert-butyl [4-(3-tert-butyl-5-amino-1H-pyrazol-1-yl)-2-methoxybenzylcarbamate (2.23 g, 79%) as a light yellow solid. ¹H NMR (CDCl₃): δ 7.4 (m, 5H), 5.6 (s, 1H), 4.4 (d, 2H), 1.5 (s, 9H), 1.3 (s, 9H).

### EXAMPLE 43

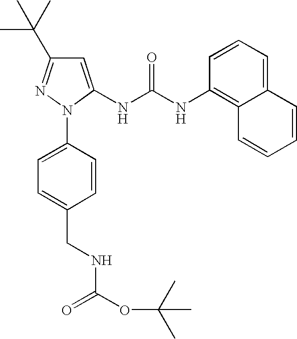

A 40 mL vial was equipped with a septum, a stir bar and a source of Ar, and charged with Example V (2 g, 5.81 mmol), flushed with Ar and dissolved in CHCl₃ (20 mL). The solution was treated with 2-naphthylisocyanate (984 mg, 5.81 mmol) in CHCl₃ (5 mL) and added over 1 min The reaction was stirred for 8 h, and additional 1-naphthylisocyanate (81 mg) was added and the reaction stirred overnight. The solid was filtered and washed with CH₂Cl₂ to yield tert-butyl 4-[3-tert-butyl-5-(3-naphthalen-1-yl)ureido)-1H-pyrazol-1-yl]benzylcarbamate (1.2 g). HPLC purity: 94.4%; ¹H NMR (DMSO-d₆): δ 9.1 (s, 1H), 8.8 (s, 1H), 8.0 (m, 3H), 7.6 (m, 9H), 6.4 (s, 1H), 4.2 (d, 2H), 1.4 (s, 9H), 1.3 (s, 9H).

### EXAMPLE 44

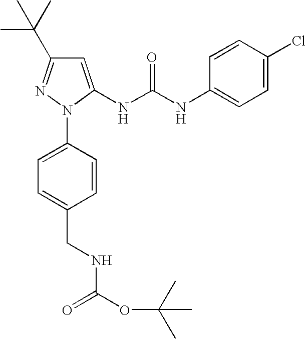

The title compound was synthesized in a manner analogous to Example 43 utilizing Example V (2.0 g, 5.81 mmol) and p-chlorophenylisocyanate (892 mg) to yield tert-butyl 4-[3-tert-butyl-5-(3-(4-chloropnehyl)ureido)-1H-pyrazol-1-yl]benzylcarbamate (1.5 g). HPLC purity: 97%; ¹H NMR (DMSO-d₆): δ 9.2 (s, 1H), 8.4 (s, 1H), 7.4 (m, 8H), 6.4 (s, 1H), 4.2 (d, 2H), 1.4 (s, 9H), 1.3 (s, 9H).

### EXAMPLE 45

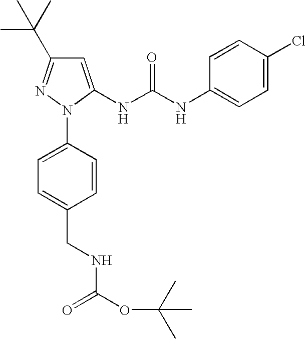

A 10 mL flask equipped with a stir bar was flushed with Ar and charged with Example 43 (770 mg, 1.5 mmol) and CH₂Cl₂ (1 ml) and 1:1 CH₂Cl₂:TFA (2.5 mL). After 1.5 h, reaction mixture was concentrated in vacuo, the residue was dissolved in EtOAc (15 mL), washed with saturated NaHCO₃ (10 mL) and saturated NaCl (10 mL). The organic layers was dried, filtered and concentrated in vacuo to yield 1-{3-tert-butyl-1-[4-(aminomethyl)phenyl]-1H-pyrazol-5-yl}-3-(naphthalen-1-yl)urea (710 mg). ¹H NMR (DMSO-d₆): δ 7.4 (m, 11H), 6.4 (s, 1H), 3.7 (s, 2H), 1.3 (s, 9H).

### EXAMPLE 46

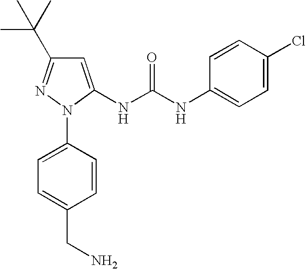

The title compound was synthesized in a manner analogous to Example 45 utilizing Example 44 (1.5 g, 1.5 mmol) to yield 1-{3-tert-butyl-1-[4-(aminomethyl)phenyl]-1H-pyrazol-5-yl}-3-(4-chlorophenyl)urea (1.0 g). HPLC purity: 93.6%; mp: 100-102; ¹H NMR (CDCl₃): δ 8.6 (s, 1H), 7.3 (m, 8H), 6.3 (s, 1H), 3.7 (brs, 2H), 1.3 (s, 9H).

### EXAMPLE 47

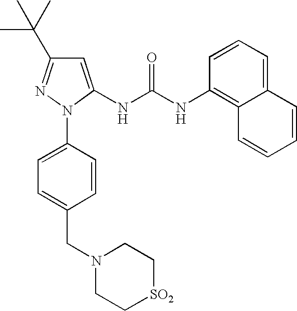

A 10 ml vial was charged with Example 45 (260 mg, 63 mmol) and absolute EtOH (3 mL) under Ar. Divinylsulfone (63 uL, 74 mg, 0.63 mmol) was added drop wise over 3 min and the reaction was stirred at RT for 1.5 h. and concentrated in vacuo to yield a yellow solid, which was purified via preparative TLC, developed in 5% MeOH:CH₂Cl₂. The predominant band was cut and eluted off the silica with 1:1 EtOAc:MeOH, filtered and concentrated in vacuo to yield 1-{3-tert-butyl-1-[4-(1,1-dioxothiomorpholin-4-yl)methylphenyl]-1H-pyrazol-5-yl}-3-(naphthalen-1-yl)urea (150 mg). HPLC purity: 96%; ¹H NMR (DMSO-d₆): δ 9.1 (s, 1H), 9.0 (s, 1H), 7.9 (m, 3H), 7.5 (m, 8H), 6.4 (s, 1H), 3.1 (brs, 4H), 2.9 (brs, 4H), 1.3 (s, 9H).

### EXAMPLE 48

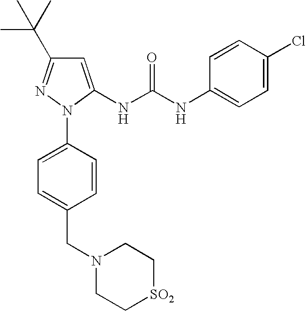

The title compound was synthesized in a manner analogous to Example 47 utilizing Example 46 (260 mg, 0.66 mmol) to yield 1-{3-tert-butyl-1-[4-(1,1-dioxothiomorpholin-4-yl)methylphenyl]-1H-pyrazol-5-yl}-3-(4-chlorophenyl)urea (180 mg). HPLC purity: 93%; mp: 136-138; ¹H NMR (DMSO-d₆): δ 9.2 (s, 1H), 8.5 (s, 1H), 7.4 (m, 9H), 6.4 (s, 1H), 3.1 (brs, 4H), 3.0 (brs, 4H), 1.3 (s, 9H).

### EXAMPLE 49

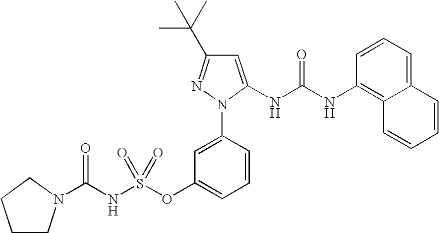

To a stirring solution of chlorosulfonyl isocyanate (0.35 g, 5 mmol) in CH₂Cl₂ (20 mL) at 0° C. was added pyrrolidine (0.18 g, 5 mmol) at such a rate that the reaction temperature did not rise above 5° C. After stirring for 2 h, a solution of Example 41 (1.10 g, 6.5 mmol) and triethylmine (0.46 g, 9 mmol) in CH₂Cl₂ (20 mL) was added. When the addition was complete, the mixture was allowed to warm to RT and stirred overnight. The reaction mixture was poured into 10% HCl (10 mL) saturated with NaCl, the organic layer was separated and the aqueous layer extracted with ether (20 mL). The combined organic layers were dried (Na₂SO₄) and concentrated in vacuo, purified by preparative HPLC to yield (pyrrolidine-1-carbonyl)sulfamic acid 3-[3-tert-butyl-5-(3-naphthalen-1-yl-ureido)-pyrazol-1-yl]phenyl ester (40 mg). ¹H NMR (CDCl₃): δ 9.12 (brs, 1H), 8.61 (brs, 1H), 7.85-7.80 (m, 3H), 7.65 (d, J=8.0 Hz, 2H), 7.53-7.51 (m, 1H), 7.45-7.25 (m, 5H), 6.89 (s, 4H), 3.36-3.34 (brs, 1H), 3.14-3.13 (brs, 2H), 1.69 (brs, 2H), 1.62 (brs, 2H), 1.39 (s, 9H); MS (ESI) m/z: 577 (M+H⁺).

### EXAMPLE 50

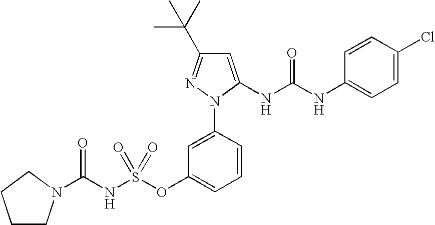

The title compound was synthesized in a manner analogous to Example 49 utilizing Example 42 to yield (pyrrolidine-1-carbonyl)sulfamic acid 3-[3-tert-butyl-5-(4-chlorophenyl-1-yl-ureido)pyrazol-1-yl]phenyl ester. MS (ESI) m/z: 561 (M+H⁺).

### EXAMPLE W

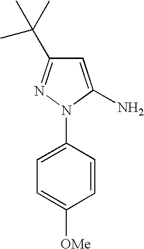

Solid 4-methoxyphenylhydrazine hydrochloride (25.3 g) was suspended in toluene (100 mL) and treated with triethylamine (20.2 g). The mixture was stirred at RT for 30 min and treated with pivaloylacetonitrile (18 g). The reaction was heated to reflux and stirred overnight. The hot mixture was filtered, the solids washed with hexane and dried in vacuo to afford 3-tert-butyl-1-(4-methoxyphenyl)-1H-pyrazol-5-amine (25 g, 70%). ¹H NMR (DMSO-d₆): δ 7.5 (d, 2H), 7.0 (d, 1H), 6.4 (s, 1H), 6.1 (s, 211), 3.9 (s, 311), 1.3 (s, 9H).

### EXAMPLE 51

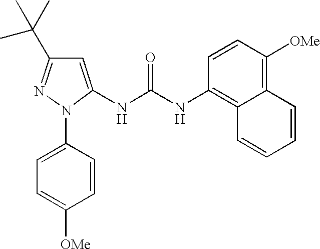

To a solution of 1-isocyanato-4-methoxy-naphthalene (996 mg) in anhydrous CH₂Cl₂ (20 mL) of was added Example W (1.23 g). The reaction solution was stirred for 3 h, the resulting white precipitate filtered, treated with 10% HCl and recrystallized from MeOH, and dried in vacuo to yield 1-[3-tert-butyl-1-(4-methoxyphenyl)-1H-pyrazol-5-yl]-3-(1-methoxynaphthalen-4-yl-urea as white crystals (900 mg, 40%). HPLC purity: 96%; mp: 143-144; ¹H NMR (DMSO-d₆): δ 8.8 (s, 1H), 8.5 (s, 1H), 8.2 (d, 1H), 8.0 (d, 1H), 7.6 (m, 5H), 7.1 (d, 2H), 7.0 (d, 1H), 6.3 (s, 1H), 4.0 (s, 3H), 3.9 (s, 3H); 1.3 (s, 9H).

### EXAMPLE 52

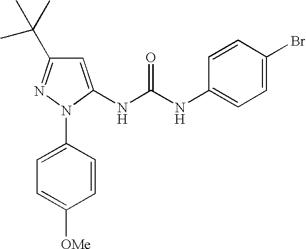

The title compound was synthesized in a manner analogous to Example 51 utilizing Example W and p-bromophenylisocyanate (990 mg) to yield 1-{3-tert-butyl-1-(4-methoxyphenyl)-1H-pyrazol-5-yl}-3-(4-bromophenyl)urea as off-white crystals (1.5 g, 68%). HPLC purity: 98%; mp: 200-201; ¹H NMR (DMSO-d₆): 69.3 (s, 1H), 8.3 (s, 1H), 7.4 (m, 6H), 7.0 (d, 2H), 6.3 (s, 1H), 3.8 (s, 3H), 1.3 (s, 9H).

### EXAMPLE 53

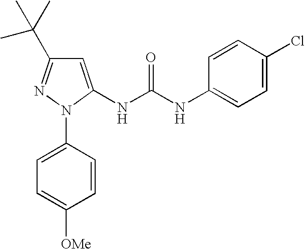

The title compound was synthesized in a manner analogous to Example 51 utilizing Example W and p-chlorophenylisocyanate (768 mg) into yield 1-{3-tert-butyl-1-(4-methoxyphenyl)-1H-pyrazol-5-yl}-3-(4-chlorophenyl)urea as white crystals (1.3 g, 65%). HPLC purity: 98%; mp: 209-210; ¹H NMR (DMSO-d₆): δ 9.1 (s, 1H), 8.3 (s, 1H), 7.4 (m, 4H), 7.3 (d, 2H), 7.1 (d, 2H), 6.3 (s, 1H), 3.8 (s, 3H), 1.3 (s, 9H).

### EXAMPLE 54

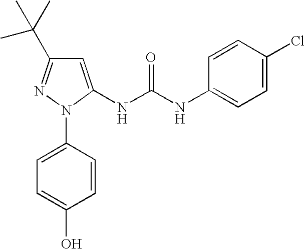

The title compound was synthesized in a manner analogous to Example 41 utilizing Example 53 (500 mg) to yield 1-{3-tert-butyl-1-(4-hydroxyphenyl)-1H-pyrazol-5-yl}-3-(4-chlorophenyl)urea as white crystals (300 mg, 62%). HPLC purity: 94%; mp: 144-145; ¹H NMR (DMSO-d₆): δ 9.7 (s, 1H), 9.1 (s, 1H), 8.3 (s, 1H), 7.4 (d, 2H), 7.3 (m, 4H); 6.9 (d, 2H), 6.3 (s, 1H), 1.3 (s, 9H)

### EXAMPLE 55

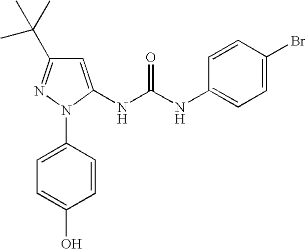

The title compound was synthesized in a manner analogous to Example 41 utilizing Example 52 (550 mg) to yield 1-{3-tert-butyl-1-(4-hydroxyphenyl)-1H-pyrazol-5-yl}-3-(4-bromophenyl)urea as a white crystalline solid (400 mg, 70%). HPLC purity: 93%; mp: 198 200

¹H NMR (DMSO-d₆): δ 9.7 (s, 1H), 9.2 (s, 1H), 8.3 (s, 1H), 7.4 (d, 4H), 7.2 (m, 2H), 6.9 (d, 2H), 6.3 (s, 1H), 1.3 (s, 9H).

### EXAMPLE X

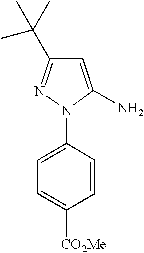

Methyl 4-(3-tert-butyl-5-amino-1H-pyrazol-1-yl)benzoate (3.67 mmol) was prepared from methyl 4-hydrazinobenzoate and pivaloylacetonitrile by the procedure of Regan, et al., J. Med. Chem., 45, 2994 (2002).

### EXAMPLE 56

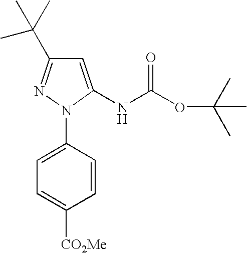

A 500 mL round bottom flask was equipped with a magnetic stir bar and an ice bath. The flask was charged with Example X (1 g) and this was dissolved in CH₂Cl₂ (100 mL). Saturated sodium bicarbonate (100 mL) was added and the mixture rapidly stirred, cooled in an ice bath and treated with diphosgene (1.45 g) and the heterogeneous mixture stirred for 1 h. The layers were separated and the CH₂Cl₂ layer treated with tert-butanol (1.07 g) and the solution stirred overnight at RT. The solution was washed with H₂O (2×150 mL), dried (Na₂SO₄), filtered, concentrated in vacuo, and purified by flash chromatography using 1:2 ethyl acetate:hexane as the eluent to yield tert-buthyl 1-(4-(methoxycarbonyl)phenyl)-3-tert-butyl-1H-pyrazol-5-ylcarbamate (100 mg) as an off-white solid. ¹H NMR (DMSO-d₆): δ 9.2 (s, 1H), 8.1 (d, 2H), 7.7 (d, 2H), 6.3 (s, 1H), 3.3 (s, 3H), 1.3 (s, 18H).

### EXAMPLE 57

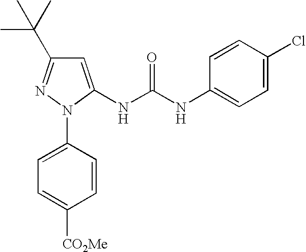

The title compound was synthesized in a manner analogous to Example 41 utilizing Example X (1.37 g) and p-chlorophenylisocyanate (768 mg) to yield methyl 4-{3-tert-butyl-5-[3-(4-chlorophenyl)ureido]-1H-pyrazol-1-yl}benzoate as white crystals (1.4 g 66%). HPLC purity: 98%; mp: 160-161; ¹H NMR (DMSO-d₆): δ 9.2 (s, 1H), 8.6 (s, 1H), 8.1 (d, 2H), 7.8 (d, 2H), 7.5 (d, 2H), 7.3 (d, 2H), 6.4 (s, 1H), 3.9 (s, 3H), 1.3 (s, 9H).

### EXAMPLE 58

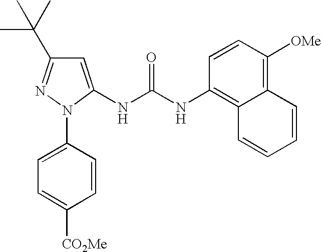

The title compound was synthesized in a manner analogous to Example 41 utilizing Example X (1.27 g) and 1-isocyanato-4-methoxy-naphthalene (996 mg) to yield methyl 4-{3-tert-butyl-5-[3-(1-methoxynaphthalen-4-yl)ureido]-1H-pyrazol-1-yl}benzoate as white crystals (845 mg, 36%). HPLC purity: 98%; mp: 278 280; ¹H NMR (DMSO-d₆): δ 8.76 (s, 1H), 8.73 (s, 1H), 8.1 (m, 3H), 7.9 (d, 1H), 7.7 (d, 2H), 7.6 (m, 3H), 7.0 (d, 1H), 7.0 (d, 1H), 6.3 (s, 1H), 4.0 (s, 3H), 3.9 (s, 3H),1.3 (s, 9H).

### EXAMPLE 59

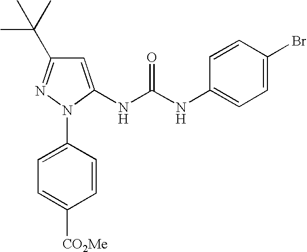

The title compound was synthesized in a manner analogous to Example 41 utilizing Example X (1.37 g) and p-bromophenylisocyanate (990 mg) to yield methyl 4-{3-tert-butyl-5-[3-(4-bromophenyl)ureido]-1H-pyrazol-1-yl}benzoate as white crystals (1.4 g, 59%). HPLC purity: 94%; mp: 270 272; ¹H NMR (DMSO-d₆): δ 9.2 (s, 1H), 8.6 (s, 1H), 8.1 (d, 2H), 7.7 (d, 2H), 7.4 (d, 4H), 6.4 (s, 1H), 3.9 (s, 3H), 1.3 (s, 9H).

### EXAMPLE 60

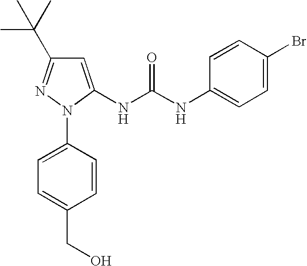

To a solution of Example 59 (700 mg) in 30 mL of toluene at −78° C., was added dropwise a solution of diisobutylaluminum hydride in toluene (1M in toluene, 7.5 mL) over 10 min. The reaction mixture was stirred for 30 min at −78° C., and then 30 min at 0° C. The reaction mixture was concentrated in vacuo to dryness and treated with H₂O. The solid was filtered and treated with acetonitrile. The solution was evaporated to dryness and the residue was dissolved in ethyl acetate, and precipitated by hexanes to afford yellow solid which was dried under vacuum to give 1-[3-tert-butyl-1-(4-hydroxymethyl)phenyl)-1H-pyrazol-5-yl]urea (400 mg, 61%). HPLC purity: 95%; ¹H NMR (DMSO-d₆): 69.2 (s, 1H), 8.4 (s, 1H), 7.5 (m, 8H), 6.4 (s, 1H), 5.3 (t, 1H), 4.6 (d, 2H), 1.3 (s, 9H).

All of the references above identified are incorporated by reference herein. In addition, two simultaneously filed applications are also incorporated by reference, namely Anti-Inflammatory Medicaments, S/N ______, filed ______ and Anti-Cancer Medicaments, S/N ______ filed ______.

We claim:

1. A method of identifying molecules which interact with specific naturally occurring proteins in order to regulate the activity of the proteins, said method comprising the steps of:
identifying a switch control ligand forming a part of said protein;
identifying a switch control pocket forming a part of said protein and which interacts with said switch control ligand,
said ligand interacting in vivo with said pocket to regulate the conformation and biological activity of said protein such that the protein will assume a first conformation and a first biological activity upon said ligand-pocket interaction, and will assume a second, different conformation and biological activity in the absence of said ligand-pocket interaction;
providing respective samples of said protein in said first and second conformations; and
screening at least one of said samples against one or more candidate molecules by contacting the molecules and one said sample, and identifying small molecules which bind with such protein at the region of said pocket in order to regulate the activity of the protein.

2. The method of claim 1, said protein selected from the group consisting of enzymes, receptors, and signaling proteins.

3. The method of claim 2, said protein selected from the group consisting of kinases, phosphotases, sulfotranferases, sulfatases, transcription factors, nuclear hormone receptors, g-protein coupled receptors, g-proteins, gtp-ases, hormones, polymerases, and other proteins containing nucleotide regulatory sites.

4. The method of claim 1, said protein having a molecular weight of at least about 15 kDa.

5. The method of claim 4, said molecular weight being above about 30 kDa.

6. The method of claim 1, said steps of identifying said switch control ligand sequences and said switch control pockets selected from the group consisting of analysis of bioinformatics, X-ray crystallography, nuclear magnetic resonance spectroscopy (NMR), circular dichroism (CD), and affinity base screening.

7. The method of claim 1, said protein-providing step comprising the step of obtaining substantially purified samples of said protein statically confined to respective states corresponding to said first and second conformations.

8. The method of claim 1, said contacting step comprising a technique selected from the group consisting of affinity-based screening, capillary zone electrophoresis, fluoroprobe displacement assay, nuclear magnetic resonance spectroscopy, circular dichroism, and X-ray crystallography.

9. The method of claim 1, said protein being a kinase protein.

10. A protein-modulator adduct comprising a naturally occurring protein having a switch control pocket with a non-naturally occurring molecule bound to the protein at the region of said switch control pocket, said molecule serving to at least partially regulate the biological activity of said protein by inducing or restricting the conformation of the protein.

11. The adduct of claim 10, said molecule serving to induce a conformation change in said protein.

12. The adduct of claim 10, said molecule serving to restrict a conformation change in said protein.

13. The adduct of claim 10, said protein also having a switch control ligand, said ligand interacting in vivo with said pocket to regulate the conformation and biological activity of said protein such that the protein will assume a first conformation and a first biological activity upon said ligand-pocket interaction, and will assume a second, different conformation and biological activity in the absence of said ligand-pocket interaction.

14. The adduct of claim 10, said pocket being an on-pocket, said molecule binding with said protein at the region of said on-pocket as an agonist.

15. The adduct of claim 10, said pocket being an on-pocket, said molecule binding with said protein at the region of said on-pocket as an antagonist.

16. The adduct of claim 10, said pocket being an off-pocket, said molecule binding with said protein at the region of said off-pocket as an agonist.

17. The adduct of claim 10, said pocket being an off-pocket, said molecule binding with said protein at the region of said off-pocket as an antagonist.

18. The adduct of claim 10 said protein selected from the group consisting of enzymes, receptors, and signaling proteins.

19. The adduct of claim 18, said protein selected from the group consisting of kinases, phosphotases, sulfotranferases, sulfatases, transcription factors, nuclear hormone receptors, g-protein coupled receptors, g-proteins, gtp-ases, hormones, polymerases, and other proteins containing nucleotide regulatory sites.

20. The adduct of claim 10, said protein having a molecular weight of at least about 15 kDa.

21. The adduct of claim 20, said molecular weight being above about 30 kDa.

22. The adduct of claim 10, said protein being a kinase protein.

23. A method of altering the biological activity of a protein comprising the steps of:
providing a naturally occurring protein having a switch control pocket;
contacting said protein with a non-naturally occurring molecule modulator; and
causing said modulator to bind with said protein at the region of said pocket in order to at least partially regulate the biological activity of the protein by inducing or restricting the conformation of the protein.

24. The method of claim 23, said molecule serving to induce a conformation change in said protein.

25. The method of claim 23, said molecule serving to restrict a conformation change in said protein.

26. The method of claim 23, said protein also having a switch control ligand, said ligand interacting in vivo with said pocket to regulate the conformation and biological activity of said protein such that the protein will assume a first conformation and a first biological activity upon said ligand-pocket interaction, and will assume a second, different conformation and biological activity in the absence of said ligand-pocket interaction.

27. The method of claim 23, said pocket being an on-pocket, said molecule binding with said protein at the region of said on-pocket as an agonist.

28. The method of claim 23, said pocket being an on-pocket, said molecule binding with said protein at the region of said on-pocket as an antagonist.

29. The method of claim 23, said pocket being an off-pocket, said molecule binding with said protein at the region of said off-pocket as an agonist.

30. The method of claim 23, said pocket being an off-pocket, said molecule binding with said protein at the region of said off-pocket as an antagonist.

31. The method of claim 23 said protein selected from the group consisting of enzymes, receptors, and signaling proteins.

32. The method of claim 31, said protein selected from the group consisting of kinases, phosphotases, sulfotranferases, sulfatases, transcription factors, nuclear hormone receptors, g-protein coupled receptors, g-proteins, gtp-ases, hormones, polymerases, and other proteins containing nucleotide regulatory sites.

33. The method of claim 32, said protein having a molecular weight of at least about 15 kDa.

34. The method of claim 33, said molecular weight being above about 30 kDa.

35. The method of claim 32, said protein being a kinase protein.
